(19) United States
(12) Patent Application Publication
(10) Pub. No.: US 2018/0284130 A1
(43) Pub. Date: Oct. 4, 2018

(54) BINDING ASSAYS AND METHOD FOR PROBING ANTIBODY FUNCTION WITH FC BINDING MULTIMERS

(71) Applicant: THE MACFARLANE BURNET INSTITUTE FOR MEDICAL RESEARCH AND PUBLICHEALTH LTD, Melbourne, Victoria (AU)

(72) Inventors: Phillip Mark HOGARTH, Williamstown, Victoria (AU); Bruce David WINES, Heidelberg, Victoria (AU)

(21) Appl. No.: 15/765,171

(22) PCT Filed: Oct. 4, 2016
(86) PCT No.: PCT/AU2016/000337

§ 371 Date: Mar. 30, 2018

(30) Foreign Application Priority Data
Oct. 2, 2015 (AU) 2015904018

(51) Int. Cl. G01N 33/68; G01N 33/543

(52) U.S. Cl. CPC G01N 33/6854; G01N 33/54306; G01N 33/6845

(57) ABSTRACT

The present disclosure relates to the use of an at least one soluble Fc binding multimer comprising at least two Fc binding regions to assess FcR binding activity of an at least one polypeptide comprising an Fc region or fragment thereof, including an antibody, and methods and assays of using the Fc binding multimer to assess FcR binding activity. The FcR binding multimer may be a fusion polypeptide comprising an at least two Fc binding regions, or may consist of at least two peptides each comprising at least one Fc binding region, that are covalently or non-covalently oligomerised.

### TECHNICAL FIELD

The present disclosure relates to the use of a multimer that can bind an antibody. In a particular form, the multimer comprises at least two Fc binding regions.

### BACKGROUND

Although the antigen specificity of antibody (or immunoglobulin) is determined by the antigen binding (Fab) domains, the effector function of an antibody is determined by the Fc region (also referred to as the Fc domain, Fc portion or, simply, Fc). Antibodies bind to cells through cell bound Fc receptors, which are present on the surface of certain cells, such as B lymphocytes, follicular dendritic cells, dendritic cells, some T cells (eg γδ T cells), natural killer cells, macrophages, neutrophils, eosinophils, basophils and mast cells and also platelets. During antibody-mediated immune responses, an antibody may bind to a specific antigen (eg a pathogenic antigen, cancer cell or virally infected cell) through the antigen binding (Fab) domains forming an immune complex (IC), and the Fc region of an antibody in an immune complex can then be captured by a cellular Fc receptor (FcR; Fc binding region). FcR on cells may be cross-linked upon the binding of multiple IgG molecules (eg as found in an immune complex) to the cell, which may activate the cells to produce immune system effector functions such as antibody-dependent cellular phagocytosis (ADCP), antibody-dependent cellular cytotoxicity (ADCC), cytokine release, respiratory burst, degranulation or antigen presentation.

The administration to a patient of therapeutic antibodies that can modulate immune function has revolutionised the treatment of diseases including blood cancers and solid tumours (eg monoclonal antibody therapy). The effect of the antibody, for example, anti-tumour activity in the case of the monoclonal antibodies known as rituximab and trastuzumab, is considered at least in part to be FcR dependent.

Different types of FcR bind preferentially with different types of immunoglobulin (Ig). For example, the FcR that bind to IgG are termed FcγR, which includes FcγRI (CD64), FcγRII (CD32) and FcγRIII (CD16). The different molecular structures of the various FcγR are associated with different binding affinities for different isotypes of IgG, for example, the Fc binding affinity of FcγRI is high (particularly for IgG1 and IgG3, including in the monomeric form). In contrast, the Fc binding affinity of the FcγRII and FcγRIII isotypes for IgG is low. Indeed, it is thought that FcγRIIa and FcγRIIIa may only bind to multimeric antigen-antibody complexes (ie immune complexes). Moreover, the activation of different FcR may activate different immune functions. For example, FcγRIIb is an inhibitory receptor, whereas the other FcγR are activating receptors. This is further complicated by cell type, with a particular FcγR mediating different effector functions on different cell types.

IgG interactions with FcγRs also vary with known functional polymorphisms of the receptors. For example, the Val158 polymorphism of FcγRIIIa has stronger affinity for IgG than the Phe158 allelic form (eg Bruhns et al., 2009) and is associated with a greater capacity for eliciting ADCC). Further, patients with the Val158 high binding activity allele have been reported to exhibit a superior clinical response to therapeutic IgG treatments (Hogarth and Pietersz, 2012). These studies and others have established the principle that the efficacy of FcR interactions is causal of the activation of leukocyte effector cells that mediate immunity and forms a key mechanism of action that underlies clinical outcomes to therapeutic antibodies.

The glycosylation state of the Fc and mutations in the FcR may further affect binding activity. For example, FcγRIIIa is expressed on natural killer cells and some monocytes, where it performs a key role in killing or phagocytosis of antibody-opsonised targets by these cells. Also, there have been reports that therapeutic responses to anti-tumour IgG1 antibodies correlated with patient expression of high affinity (Val158) alleles of FcγRIIIa, thereby demonstrating the importance of Fc-FcγRIII interaction in the mechanism of action of these antibodies (Weng and Levy, 2003; Musolino et al., 2008). Further, analysis of the glycosylation of the Fc found that the absence of a bisecting fucose in the Asn297-linked carbohydrate greatly increased interaction of the Fc with FcγRIIIa (Ferrara et al., 2006; Ferrara et al., 2011; Shinkawa et al., 2003). Thus, therapeutic antibodies that lack this fucose have been shown to have improved receptor binding and target killing functions in pre-clinical studies (Lida et al., 2006; Herter et al., 2013) and were associated with improved outcomes in the clinic. In another example, FcγRIIa has been shown to be involved in neutrophil killing of tumour targets and this functionality has been found to be reduced when the therapeutic antibody used was optimised for FcγRIIIa interaction alone without equivalent improvement in the interaction with FcγRIIa (Derer et al., 2014). Random and structure-guided mutagenesis of the Fc has led to the development of antibodies with attenuated or enhanced activities for engaging activating or inhibitory Fc receptors (eg An et al., 2009; Baruah et al., 2012). However, differences in IgG epitope recognition within the same target antigen, such as that exhibited by type I and II anti-CD20 antibodies, can markedly influence their functional properties such as complement dependent killing and intrinsic cell death mechanisms (Klein et al., 2013).

Accordingly, in addition to the specific antigen binding capabilities, the interaction between a therapeutic antibody and Fc receptors should be considered when screening therapeutic antibodies, as this interaction may profoundly affect the resulting immune system effector functions such as ADCC and ADCP. However, since cell-based assays for measuring cellular activity, such as phagocytosis and antibody dependent cellular cytotoxicity, are difficult to standardise, there is a need to provide improved tools to test the binding efficacy between FcR and antibodies (eg antibodies such as those produced in vitro including engineered antibodies), in addition to evaluating the quality of antibodies raised in vivo, for example, in response to vaccination or naturally acquired immunity.

### SUMMARY

In an aspect, the present disclosure relates to the use of an at least one soluble Fc binding multimer comprising at least two Fc binding regions to assess FcR binding activity of an at least one polypeptide comprising an Fc region or fragment thereof comprising an Fc region or fragment thereof. In an embodiment, the polypeptide comprising an Fc region or fragment thereof is an antibody.

In another aspect, the present disclosure relates to a method of assessing in vitro the FcR binding activity of an antibody comprising an Fc region or fragment thereof, the method comprising the following steps:

a. providing an at least one Fc binding multimer comprising at least two Fc binding regions;
b. incubating the antibody with the at least one Fc binding multimer under conditions suitable to permit specific binding of the Fc binding regions to a Fc region or fragment thereof; and
c. identifying the magnitude of FcR binding activity of the antibody with the Fc binding multimer using a label agent;
wherein the magnitude of FcR binding activity identified in step (c) provides an assessment of the FcR binding activity of the antibody.

In an embodiment, the Fc binding multimer comprises at least two homologous Fc binding regions. In an embodiment, the Fc binding regions are derived from an Fcγ receptor (FcγR) selected from the group consisting of FcγRIIa-His131 FcγRIIa-Arg131, FcγRIIa-Pro131, FcγRIIb, FcγRIIIa-Val158, and FcγRIIIa-Phe158.

In an embodiment, the Fc binding multimer consists of an Fc binding peptide comprising at least two Fc binding regions joined by a peptide linker domain. In an embodiment, the Fc binding multimer consists of at least two Fc binding peptides each comprising an at least one Fc binding region, wherein each Fc binding peptide is bound to a first partner of a specific binding pair such that when a second partner of the specific binding pair is present, at least two of the first partners each bind with the second partner of the specific binding pair to form the Fc binding multimer.

In an embodiment, assessing FcR binding activity between the antibody and the Fc binding multimer identifies which Fc receptors (FcRs) the antibody preferentially binds with, wherein the identifying of preferential binding to an FcR indicates whether the antibody will in use induce a desired immune response outcome.

In an embodiment, step (b) further comprises immobilising the antibody by binding with an immobilised specific antigen or an immobilised immunoglobulin or fragment thereof which specifically binds with the antibody, prior to incubating the antibody with the at least one Fc binding multimer. In an embodiment, the step (b) further comprises incubating the antibody with a cell bound specific antigen, or a cell-fragment-bound specific antigen to bind the antibody to the cell or cell fragment, prior to incubating the antibody with the at least one Fc binding multimer.

In an embodiment, the method further comprises comparing the magnitude of FcR binding activity between the Fc binding multimer and the antibody with the magnitude of binding between the Fc binding multimer and one or more control antibodies.

In an embodiment, the increased magnitude of FcR binding activity of the antibody compared to a control antibody demonstrates that the Fc region of the antibody has stronger binding activity for the Fc binding region than the control antibody, a decreased magnitude of binding of the antibody compared to a control antibody demonstrates that the antibody has weaker binding activity for the Fc binding region than the control antibody, or unchanged or similar magnitude of binding of the Fc region of an antibody compared to a control antibody demonstrates that the antibody has the same or similar binding activity for the Fc binding region as the control antibody.

In an embodiment, the method further comprises comparing the magnitude of FcR binding activity between the antibody and a plurality of different Fc binding multimers to identify which Fc binding regions the antibody preferentially binds with, and wherein the identifying of preferential binding to a Fc binding region indicates whether the antibody will in use induce a desired immune response outcome.

In an embodiment, the method further comprises comparing the magnitude of FcR binding activity between the Fc binding multimer and a panel of different antibodies each specific for a same specific antigen to identify an antibody that preferentially binds to a Fc binding multimer comprising at least two Fc binding regions derived from a selected FcR.

In an embodiment, the FcR binding activity of at least two target antibodies are simultaneously assessed to map the proximity of the at least two antibodies when bound to a target antigen.

In an embodiment, step (b) further comprises incubating the antibody with an antigenic target or target cell under conditions suitable to permit opsonisation of the antigenic target or target cell with the antibody, and then incubating the antibody with the at least one Fc binding multimer, wherein the FcR binding activity of the antibody indicates whether opsonisation of the antigenic target or target cell with the antibody is likely to induce a cellular induced response including antibody dependent cellular cytoxicity (ADCC), antibody dependent cellular phagocytosis, degranulation, mediator release, cytokine production and antigen presentation, or lack thereof.

In another aspect, the present disclosure relates to a method of selecting an at least one antibody or fragment thereof having an Fc region with an optimal FcR binding activity from a panel of antibodies or fragments thereof, the method comprising

a. providing an Fc binding multimer comprising at least two Fc binding regions, wherein the Fc binding regions are derived from a pre-selected Fc receptor (FcR);
b. separately incubating each of the panel of antibodies or fragments thereof with the Fc binding multimer under conditions suitable to permit specific binding of an antibody with a Fc region having the preferred FcR binding activity with the Fc binding multimer;
c. detecting magnitude of binding of each of the panel of antibodies to the Fc binding multimer; and
d. selecting from the panel of antibodies an antibody having a high magnitude of binding with the Fc multimer;
wherein the selected antibody has the optimal FcR binding activity.

In another aspect, the present disclosure relates to an assay to assess in vitro the FcR binding activity of an antibody comprising an Fc region or fragment thereof.

### BRIEF DESCRIPTION OF FIGURES

FIG. 1 provides a schematic drawing exemplifying an embodiment of the binding assay using IgG1 showing (A) a binding assay anti-IgG control with F(ab′)₂ anti-F(ab′)₂ bound to a plate, which has specifically bound with the F(ab′) of IgG1, which in turn has specifically been bound by anti-IgG-HRP as a control; (B) binding assay for an FcγR dimer with F(ab′)₂ anti-F(ab′)₂ bound to a plate, which has specifically bound with the F(ab′) of IgG1, which in turn has specifically been bound with the biotinylated FcγR dimer, which in turn has bound with streptavidin-HRP; (C) a binding assay anti-IgG control with 2,4,6-Trinitrophenyl hapten conjugated to Bovine Serum Albumin protein (TNP-BSA) bound to a plate, which has specifically bound with anti-TNP IgG1, which in turn has specifically been bound by anti-IgG-HRP as a control; and (D) binding assay for an FcγR dimer with TNP-BSA bound to a plate, which has specifically bound with anti-TNP IgG1, which in turn has specifically been bound with the biotinylated FcγR dimer, which in turn has bound with streptavidin-HRP; in each case the amount of bound HRP would be detected using standard techniques;

FIG. 2 provides a schematic representation of the generic format of the recombinant forms of FcR described herein. (A) FcR monomer and (B) FcR multimer (dimer). In the case of the monomer FcR, the constructed recombinant DNA encodes proteins with an appropriate signal sequence and Fc binding region module (eg FcγR ectodomains) connected to an amino acid sequence that contains a tag for purification and a target for labelling such as a hexa-histidine tag and BirA ligase target (ie H6 tag BirA target). The modular arrangement of an FcR binding region and linker can be repeated two or more times to generate multimers of Fc binding regions such as dimeric and trimeric forms and so on. Thus, for example, a dimer form (shown in B) was constructed wherein a recombinant DNA encodes protein with an appropriate signal sequence and an Fc binding region module (eg FcγR ectodomains) connected to a linker which is in turn connected to a second Fc binding region (eg an identical or heterologous FcR binding region) and in turn to an amino acid sequence that contains tag for purification and a target for labelling such as a hexa-histidine tag and BirA ligase target (ie H6 tag BirA target);

FIG. 3 provides graphical representation of the binding activity of human IgG subclasses at concentrations from approximately 500 to 1 ng/ml formed into immune complexes (ICs) comprising either (A, C, E, G, I) plate bound F(ab′)₂ anti-human F(ab′)₂ myeloma complexed with IgG1, IgG2, IgG3, or IgG4 subclass antibodies or by (B, D, F, H, J) plate bound trinitrophenol (TNP) hapten conjugated to bovine serum albumin (TNP-BSA) complexed with recombinant human anti-TNP IgG1, IgG2 and IgG4 subclass antibodies; reacted with (A, B) HRP-conjugated anti-human IgG; or the following FcγR biotinylated dimers (C, D) biotinylated FcγRIIa His131 dimer, (E, F) biotinylated FcγRIIIa Val158 dimer, or (G, H) biotinylated FcγRIIIa Phe158 dimer, with subsequent detection with streptavidin-HRP; (K, L) Mean EC50 values±95% C.I., f=number of curve fits from independent experiments, n.c.=not calculated;

FIG. 4 provides graphical representation of the binding activity of human IgG subclass myeloma proteins IgG1, IgG2, IgG4 (5 μg/ml) or IgG3 (1.25 μg/ml) captured using F(ab′)₂ anti-human F(ab′)₂ to from immune complexes and reacted with the indicated dilutions of (A) HRP-conjugated anti-human IgG or the following biotinylated FcγR monomer (B) hFcγRIIIa-Phe158, (C) mnFcγRIIa-His131, (D) mnFcγRIIa-Pro131, (E) hFcγRIIa-His131, (F) hFcγRIIa-Arg131, (G) mnFcγRIIb-His131, or with (H) hFcγRIIb-Arg131, complexed (oligomerised) with streptavidin-HRP;

FIG. 5 provides graphical representation of the binding activity of serially diluted human IgG subclass myeloma proteins IgG1, IgG2, IgG4 (initially 5 μg/ml) and IgG3 (initially 1.25 μg/ml) captured using F(ab′)₂ anti-human F(ab′)₂, and then reacted with (A) HRP-conjugated anti-human IgG, or the following biotinylated FcγR monomer (B) hFcγRIIIa Phe158, (C) mnFcγRIIa His131, (D) mnFcγRIIa Pro131, (E) hFcγRIIa His131, (F) hFcγRIIa Arg131, (G) mnFcγRIIb-His131, or (H) hFcγRIIb, complexed with HRP-conjugated streptavidin;

FIG. 6 provides graphical representation of the binding activity of recombinant human IgG1, IgG2 and IgG4 subclass antibodies specific for the TNP hapten having been reacted with TNP-BSA. Binding of IgG to TNP-BSA was evaluated with (A) HRP-conjugated anti-human IgG, or the following biotinylated FcγR monomer (B) hFcγRIIa-His131, (C) hFcγRIIa-Arg131, (D) hFcγRIIb, or (E) hFcγRIIIa-Phe158, complexed with HRP-conjugated streptavidin;

FIG. 7 provides graphical representation of ELISA analysis of the interaction of dimeric rsFcγRIIb with human IgG1, IgG2 and IgG4 subclass human mAbs formed into ICs over a range of concentrations from 500 ng/ml to 1 ng/ml by reaction with TNP-BSA as described for FIG. 3; reacted with either (A) HRP-conjugated anti-human IgG or (B) with dimeric rsFcγRIIb (5 μg/ml); showing mean values (absorbance 450) of duplicates; EC₅₀ values±S.D. were derived as described in FIG. 3;

FIG. 8 provides graphical representation of the binding activity of the anti-CD20 monoclonal (mAb) rituximab (filled symbols) or the afucosylated anti-CD20 mAb obinutuzumab (unfilled symbols), captured using F(ab′)2 anti-human F(ab′)2 and reacted with (A) HRP-conjugated anti-human IgG or (B) biotinylated FcγRIIIa Val158 dimer, (C) biotinylated FcγRIIIa Phe158 dimer, followed by detection with streptavidin-HRP; rituximab (D, F) or the afucosylated anti-CD20 mAb obinutuzumab (E, G) at concentrations from 1000 to 10 ng/ml, formed into an immune complex by capture with plate bound F(ab′)2 anti-human F(ab′)2, and then reacted with (D, E) 1000 to 1 ng/ml FcγRIIIa-Phe158 monomer biotin complexed with streptavidin-HRP (filled symbols) or (F, G) 1000 to 1 ng/ml FcγRIIIa-Phe158 dimer biotin, followed by detection with streptavidin-HRP (unfilled symbols); (H) for the 1000 and 315 ng/ml antibody concentrations, the ratio (binding preference) of the FcR binding activity of obinutuzumab/rituximab is shown for FcγRIIIa-Phe158 dimer and for FcγRIIIa-Phe158 monomer complexed with streptavidin, where OD (A450 nm) values <0.1 were excluded from calculating this ratio;

FIG. 9 provides graphical representation of the binding activity of the anti-CD20 mAb rituximab (filled symbols ●) or the afucosylated anti-CD20 mAb obinutuzumab (unfilled symbols □) captured using F(ab′)₂ anti-human F(ab)₂ and reacted with (A) biotin-conjugated anti-human IgG or streptavidin (SA)-complexed FcγR biotinylated monomers (B) FcγRIIa-His131, (C) FcγRIIa-Arg131 or (D) FcγRIIb;

FIG. 10 provides graphical representation of the binding activity of Daudi cells reacted with the anti-CD20 mAbs rituximab (filled symbols ●) or obinutuzumab (unfilled symbols □) at concentrations from 10 μg/ml to 0.1 μg/ml and the opsonised cells were then reacted with (A) FITC-conjugated anti-human IgG Fc or with the following FcγR dimers (B) biotinylated FcγRIIa His131 dimer, (C) biotinylated FcγRIIIa Val158 dimer or (D) biotinylated FcγRIIIa Phe158 dimer. The specific activities (ie FcR bound/IgG bound) were calculated for each antibody concentration as a relative measure of binding stoichiometry for (E) FcγRIIIa Val158 dimer (ie MFI values in panel C/values in panel A) and (F) FcγRIIIa Phe158 dimer (ie MFI values in panel DA/values in panel A);

FIG. 11 provides graphical representation of the binding activity of Daudi cells reacted with the anti-CD20 mAbs rituximab (filled symbols ●) or obinutuzumab (unfilled symbols □) at concentrations from 10 μg/ml to 0.1 μg/ml and the opsonised cells were then reacted with (A) FITC-conjugated anti-human IgG Fc or the following streptavidin (SA) complexed FcγRII monomers (B) biotinylated FcγRIIa His131, (C) biotinylated FcγRIIa Arg131, (D) biotinylated FcγRIIb or (E) biotinylated FcγRIIa His131 dimer;

FIG. 12 provides graphical representation of the binding activity of normal mAb b12 (circles), afucosyl-b12 (triangles) and LALA mutant b12 (squares) captured using F(ab′)₂ anti-human F(ab′)₂ to from immune complexes and reacted with (A) HRP-conjugated anti-human IgG or (B) streptavidin oligomerised monomer FcγRIIa Arg131, (C) streptavidin oligomerised monomer FcγRIIa His131, (D) dimer FcγRIIa His131, (E) streptavidin oligomerised monomer FcγRIIb, (F) dimer FcγRIIIa Val158, (G) dimer FcγRIIIa Phe158, or (H) streptavidin oligomerised monomer FcγRIIIa Phe158;

FIG. 13 provides graphical representation of the binding activity of normal mAb 2G12, a domain swapped IgG1 (circles), I19R, a revertant mutant with typical IgG1 topology (triangles) and 2G12 formatted with IgG3 constant region (squares) captured using F(ab′)₂ anti-human F(ab′)₂ to from immune complexes and reacted with of (A) HRP-conjugated anti-human IgG or the following human FcγRs (as streptavidin oligomerised monomers, or when indicated as dimers), (B) streptavidin oligomerised monomers FcγRIIa Arg131, (C) streptavidin oligomerised monomers FcγRIIa His131, (D) dimer FcγRIIa His131, (E) streptavidin oligomerised monomers FcγRIIb, (F) dimer FcγRIIIa Val158, (G) dimer FcγRIIIa Phe158, or (H) streptavidin oligomerised monomers FcγRIIIa Phe158;

FIG. 14 provides cytograms from flow cytometric data of rituximab opsonisation and FcR dimer detection of normal circulating B lymphocytes. PBMC opsonised with 2.5 μg/ml rituximab (A-F) or diluent, FACS buffer, only (G-L). The extent of opsonisation of the cells was determined by staining with an anti-human IgG Fc antibody (A, D, G, J) and the binding of dimer FcγRIIa-His131, (B, E, H, K) or dimer FcγRIIIa-Val158 (C, F, I, L) was also determined. The fluorescence intensity is shown on the x and y axes as a 5 decade biexponential scale. The receptor dimers were used at 0.2 μg/ml. The lineage marker, CD19 was used to identify B cells (D, E, F) and revealed relatively reduced binding of FcγRIIa-His131 (E) compared to FcγRIIIa-Val158 (F). Control binding of anti IgG Fc or FcγR dimers on un-opsonised cells (G, H, I) or fluorescence of cells only (J, K, L) was also determined. MFI=mean fluorescence intensity;

FIG. 15 shows differential FcγR dimer binding to rituximab sensitised normal B cells. Rituximab was titrated (2 fold dilutions from 10 μg/ml) on PBMC. B Cells were identified by staining for the lineage marker CD19. The extent of rituximab opsonisation of cells was determined by staining of cells with anti-human IgG Fc (A). FcR dimer binding, FcγRIIa-His131 (B) was compared to FcγRIIIa-Val158 (C). The receptor dimers were used at 0.2 μg/ml;

FIG. 16 provides graphical results showing that the FcγR dimer assay is capable of defining patient populations of i) high CD16a activity and ii) low CD32a activity for anti-env antibody responses. Sera at 1/500 dilution from 15 patients with high CD4 counts (>500/μl) and 15 patients with low CD4 counts (<100/μl) were reacted with recombinant gp140 HIV AD8 envelope trimer (Center et al., 2009) coated at 1 μg/ml on an ELISA plate. Reaction with FcγRIIa (H131) dimer or FcγRIIIa (V158) dimer or anti-human IgG was as described above. This graphical representation of the data shows the relationship between A, C) FcγRIIa (H131) dimer or FcγRIIIa (V158) dimer (B, D) binding activity to anti-IgG binding activity for patients with high CD4 counts (A, B) and 15 patients with low CD4 counts (C, D);

FIG. 17 provides graphical representation of the binding activity of dimeric rsFcγRIIa H131 under varying opsonisation levels, with human anti-TNP IgG1 at concentrations from 500 to 1 ng/ml formed into ICs by binding to TNP-BSA comprising 1.9, 3.3, 4.2 or 5.1 TNP molecules per BSA molecule, where the ICs were reacted with (A) HRP-conjugated anti-human IgG1 or with (B) dimeric rsFcγRIIa H131 with subsequent detection with streptavidin-HRP; Binding data (mean±SD, n=2) were fitted using Prism software, to log (agonist) vs. response (variable slope, constraining bottom value=0, and EC50 shared, the top value was allow to vary freely): (C) the top value of the binding curves (i.e. Bmax) for anti-IgG and dimeric rsFcγRIIa were plotted against stoichiometry of TNP modification;

FIG. 18 provides graphical representation of the correlation between binding activity of dimeric rsFcγRIIIa Val158 and NK-92-FcγRIIIa Val158 cell activation, wherein ICs formed by reacting plasma from 30 individuals with plate-bound HA A/Perth/16/2009 (H3N2) antigen were diluted at 1:80, were assessed for the binding of dimeric rsFcγRIIIa Val158, and compared with IC (formed with plasmas diluted at 1:40) activation of NK-92 cells expressing FcγRIIIa Val158; the assays were correlated using non-parametric spearman analysis and fitted by linear regression;

FIG. 19 provides flow cytometric determination of antibody proximity using dimeric FcγR binding to IL-12Rβ1 transfected Ba/F3 cells opsonised with each of 9 anti-IL-12Rβ1 monoclonal antibodies (mAbs) X81-1, X81-20A, X81-34, X81-39, X81-48, X81-80, X81-101, X81-109, X81-128 or a mixture of all antibodies X81-mix; (A) is a series of FACS histograms showing the extent of opsonisation of the cells by each monoclonal antibody or the mix of antibodies detected with a biotinylated anti-mouse IgG and APC-conjugated streptavidin shown in the solid black line histograms and background control staining of cells with anti-IgG APC-streptavidin in the absence of monoclonal antibody is shown in the dashed line histograms; the fluorescence intensity is shown on the x-axis as a 5 decade log 10 scale; (B) is a series of FACS histograms showing the extent of opsonisation of the cells by each monoclonal antibody or the mix of antibodies detected with biotinylated dimeric recombinant soluble FcγRIIa and APC-conjugated streptavidin (solid black line) and the background control staining of opsonised cells with APC-conjugated streptavidin in the absence of FcγR dimer (dashed line), with the fluorescence intensity shown on the x-axis as a 5 decade biexponential 10 scale;

FIG. 20 provides (A) a series of flow cytometric histograms of binding of biotinylated dimeric recombinant soluble FcγRIIa to pairwise combinations of IL-12Rβ1 Mabs that had been used to opsonise Ba/F3 cells expressing IL-12Rβ1 detected with APC-conjugated streptavidin (solid black histogram), with background staining in the absence of FcγR dimer (dashed histogram); pairwise combinations of FcγRIIa binding are indicated with a solid black diamond; and (B) the resulting proximity map showing the relationship of different anti-IL-12Rβ1 mab epitopes, depicted as ellipses and the pairs that show FcR dimer binding are represented by as solid diamonds and correspond to the pairwise combinations indicated in (A);

FIG. 21 provides graphical representation showing (A) dimeric rsFcγRIIIa binding to ICs of intravenous immunoglobulin (IVIg) opsonized with HA of A(H1N1)pdm09 virus; with (B) hemagglutinin inhibition (HAi) titre; the arrow shows the approximate time of emergence of A(H1N1)pdm09; and (C) shows the correlation between FcR activity and HA inhibition titre using non-parametric spearman analysis; and

FIG. 22 provides graphical representation showing selective binding of dimeric but not monomeric recombinant soluble FcγR to IgG immune complexes; A) binding of recombinant soluble monomeric FcγRIIa or FcγRIIIa to the immune complexes formed from Human IgG1 (1 μg per ml) captured by binding with TNP BSA; and (B) binding of dimeric recombinant soluble FcγRIIa bound to IgG1 TNP BSA complexes (closed symbols) as formed in (A), and also to human IgG1 immune complexes formed by capture with plate bound Fab′ 2 anti-human Fab′ 2 (open symbols).

### DETAILED DESCRIPTION

A number of factors influence whether an antibody will be suitable for use as a therapeutic antibody, primarily its specificity for a target antigen, and the particular epitope within the antigen with which the antibody binds. However, the characteristics of the non-antigen binding part of the antibody can be instrumental in activating immune system effector functions. In particular, the peptide sequence in the vicinity of the Fc region of the antibody can affect which Fc receptors (FcR) the antibody will predominantly bind, and the affinity of such binding. In turn, the preferential binding of an Fc portion of an antibody to a particular FcR (ie in comparison a different FcR) can stimulate a particular immune effector response.

As such, the detection of the binding ability of an antibody for a particular FcR can be useful for assessing the FcR binding characteristics of that antibody. It may also indicate the type of immune response raised by that antibody in vivo. Accordingly, assessing the FcR binding activities during the screening of therapeutic antibodies could be particularly useful, as it may permit the identification of an antibody that will induce a desired aspect of the immune system effector functions, such as antibody-dependent cellular cytotoxicity (ADCC), phagocytosis including antibody mediated cellular phagocytosis (ADCP), or presentation of antigen in immune complexes, and may therefore enable tailoring of therapeutic antibodies to produce the desired immune system effector function. However, it can be difficult to detect the binding of antibodies to FcR that are considered to have lower binding affinities. In particular, when the Fc binding regions of low affinity FcR are produced in a monomer form (ie as a single Fc binding region), stable binding of an antibody is not readily detectable because of the low affinity for the immunoglobulin.

Disclosed herein are Fc binding multimers comprising multiple Fc binding regions (eg soluble FcR ectodomains or fragments thereof), which are each capable of binding an antibody via the Fc region of the antibody, or another peptide having an Fc region, such that the Fc binding multimer is capable of binding multiple antibodies, or other peptide having an Fc region. Accordingly, the Fc binding multimers are able to bind target antibodies (or another peptide having an Fc region) in a multivalent manner. This feature is advantageous, particularly for FcR considered to have lower binding affinities, as the multivalency of the binding can mediate detection of the binding of an antibody with a particular FcR that may otherwise have a binding affinity that is too low to be readily detected. Consequently, the Fc binding multimers may bind two or more antibodies concurrently. The binding of a Fc binding multimer to the two or more antibodies may advantageously mimic the cell surface clustering or cross linking of cell-bound FcRs that activates the FcR and initiates cell signalling.

Accordingly, in an aspect, the present disclosure relates to an at least one soluble Fc binding multimer comprising at least two Fc binding regions as described herein.

In an aspect, the present disclosure relates to the use of an at least one soluble Fc binding multimer comprising at least two Fc binding regions to assess FcR binding activity of an at least one polypeptide comprising an Fc region or fragment thereof.

The term “Fc” will be well understood by a person skilled in the art as referring to the non-antigen binding portion or “crystallisable fragment” of an immunoglobulin or antibody, or fragment thereof, through which an antibody is able to mediate effector functions, such as binding to a cellular receptor and inducing immune responses. Likewise, the term “FcR” will be well understood by a person skilled in the art to refer to a binding partner for Fc, which is typically an Fc receptor, or a soluble Fc binding fragment, which may be derived from an Fc receptor.

The term “Fc binding” and “FcR binding” as used herein are intended to refer to the specific binding between an Fc region of an immunoglobulin or antibody (or fragment thereof) and an Fc receptor or fragment thereof that is capable of binding Fc.

The term “Fc binding region” as used herein is intended to refer to the portion of a peptide molecule that is capable of specifically binding with Fc, for example, a peptide molecule derived from an FcR. Those skilled in the art will appreciate that there are various forms of FcR, such as cellular receptors FcαR, FcγR, FcδR, FcεR, and FcμR, etc, and that the Fc binding regions of such receptors typically comprise one or more FcR ectodomains, which comprise the extracellular region or domain of an FcR, and which mediates the Fc binding function of the FcR. The Fc binding region of the present disclosure encompasses the ectodomain(s) of any of the various forms of FcR that are able to bind Fc, as well as fragments of such ectodomains that are able to bind Fc, and synthetic Fc binding polypeptides that are able to bind Fc. Those skilled in the art will be able to readily identify Fc binding ectodomains of FcγR receptors as these domains as Ig-like domains belonging to the IgG domain superfamily (Hulett et al., 1994, Hulett et al., 1995, Hulett et al., 1998, and Tamm et al., 1996) and are typically characterised by “a tryptophan sandwich” (eg residues W90 and W113 of FcγRIIa) and other residues (eg in FcγRIIa; residues of the ectodomain 1 and ectodomain 2 linker, and the BC (W113-V119), C′E (F132-P137) and FG (G159-Y160) loops of ectodomain 2 (Hulett et al., 1994; Maxwell et al., 1999; Hogarth and Pietersz, 2012)). In an embodiment, the Fc binding region consists of the entire extracellular region of an FcR, that is both ectodomain 1 and 2 of the FcR (ie, both entire Ig-like domains of the FcR). In an embodiment, the Fc binding region may, for example, comprise all, or an Fc binding fragment of, an ectodomain of an FcγR receptor. The Fc binding regions of the present disclosure may be derived from FcR of any animals of commercial, scientific, medical or personal significance, such as humans, non-human primates and other mammals such as laboratory animals, livestock, exotic animals, and companion animals (including monkeys, race horses, donkeys, sheep, cattle, goats, pigs, lions, tigers, elephants, dogs, cats, rabbits, guinea pigs, rats and mice, etc). In an embodiment, the FcR are human FcR. In an embodiment, the FcR are macaque FcR.

As used herein, the term “Fc binding peptide” is intended to refer to a single peptide comprising one or more Fc binding regions.

The term “Fc binding multimer” is to be understood as referring to a Fc binding peptide or polypeptide, or a complex of covalently or non-covalently bonded Fc binding peptides or polypeptides, wherein the Fc binding multimer comprises two or more Fc binding regions as described herein. Where the term “Fc binding oligomer” is used herein, this is intended to more specifically refer to a complex of covalently or non-covalently bonded peptides or polypeptides, which each comprise two or more Fc binding regions, to distinguish from a genetic fusion peptide which comprises two or more Fc binding region.

As used herein, the term “FcR binding activity” refers to the property of an antibody (or other Fc containing peptide) to bind with an Fc binding region (eg an FcR). As detailed elsewhere herein, a particular antibody may bind with higher affinity (in vivo or in vitro) to a particular type of FcR, as compared to another type of FcR. The FcR binding activity of an antibody is characterised by which Fc binding regions it binds with highest affinity, and or by the detected magnitude of the binding of the antibody to a given Fc binding region.

The antibody may be of commercial or therapeutic interest that specifically binds with a specific target antigen of interest. In an embodiment, the antibody is a monoclonal antibody. However, in an embodiment, the antibody is derived from plasma or serum from an individual to determine the Fc binding activity of the plasma or serum.

In an embodiment, a suitable Fc binding region is derived from an FcR. Fc binding regions considered as having been “derived from” a particular FcR include Fc binding regions having an amino acid sequence which is equivalent to that of an FcR receptor as well as Fc binding regions which include one or more amino acid modification(s) of the sequence of the Fc binding region as found in an Fc receptor. Such amino acid modification(s) may include amino acid substitution(s), deletion(s), addition(s) or a combination of any of those modifications, providing the amino acid modifications do not substantially alter the biological activity of the Fc binding region relative to that of the particular FcR from which the modified sequence originates. Amino acid modification(s) of this kind will typically comprise conservative amino acid substitution(s). Exemplary conservative amino acid substitutions are provided in Table 1 below. Particular conservative amino acid substitutions envisaged are: G, A, V, I, L, M; D, E, N, Q; S, C, T; K, R, H; and P, Na-alkylamino acids. In general, conservative amino acid substitutions will be selected on the basis that they do not have any substantial effect on (a) the structure of the polypeptide backbone of the Fc binding region at the site of the substitution, (b) the charge or hydrophobicity of the polypeptide at the site of the substitution, and/or (c) the bulk of the amino acid side chain at the site of the substitution. Where an Fc binding region including one or more conservative amino acid substitution is prepared by synthesis, the Fc binding region may also include an amino acid or amino acids not encoded by the genetic code, such as γ-carboxyglutamic acid, hydroxyproline and D-amino acids.

TABLE 1
Exemplary conservative amino acid substitutions

| | Conservative Substitutions |
|---|---|
| Ala | Val*, Leu, Ile |
| Arg | Lys*, Gln, Asn |
| Asn | Gln*, His, Lys, Arg, Asp |
| Asp | Glu*, Asn |
| Cys | Ser |
| Gln | Asn*, His, Lys, |
| Glu | Asp*, c-carboxyglutamic acid (Gla) |
| Gly | Pro |
| His | Asn, Gln, Lys, Arg* |
| Ile | Leu*, Val, Met, Ala, Phe, norleucine (Nle) |
| Leu | Nle, Ile*, Val, Met, Ala, Phe |
| Lys | Arg*, Gln, Asn, ornithine (Orn) |
| Met | Leu*, Ile, Phe, Nle |
| Phe | Leu*, Val, Ile, Ala |
| Pro | Gly*, hydroxyproline (Hyp), Ser, Thr |
| Ser | Thr |
| Thr | Ser |
| Trp | Tyr |
| Tyr | Trp, Phe*, Thr, Ser |
| Val | Ile, Leu*, Met, Phe, Ala, Nle |

*indicates preferred conservative substitutions

Sequence identity percentages referred to herein are to be understood as having been calculated by comparing two nucleotide sequences or two amino acid sequences, as the case may be, using alignment achieved with the BestFit program with default settings for determining similarity (nb. BestFit uses the local homology algorithm of Smith and Waterman, 1981 to find the best segment of similarity between two sequences). From the BestFit alignment, a determination is made of the number of positions with identical amino acids, divided by the total number of amino acids in the respective sequence, and expressed as a percentage.

In an embodiment, the Fc binding multimer comprises at least two homologous Fc binding regions. By the term “homologous Fc binding region”, it is intended that the Fc binding multimer consists of two or more Fc binding regions comprising an identical or substantially identical amino acid sequence. As such, in an embodiment, the Fc binding multimer can be regarded as a homomultimer, wherein the at least two homologous Fc binding regions are derived from the same FcγR. A substantially identical amino acid may be regarded as an amino acid showing at least 95%, preferably at least 98%, amino acid sequence identity to the amino acid sequence of the other (ie comparator(s)) Fc binding regions of the Fc binding multimer. As such, in some embodiments, the homologous Fc binding regions may have a limited number of amino acid modifications which, preferably, do not substantially affect binding of Fc to the Fc binding region. In an embodiment, where the Fc binding multimer has more than two Fc binding regions, each of the Fc binding regions are homologous. However, in an embodiment, a Fc binding multimer may have heterologous Fc binding regions.

In an embodiment, the Fc binding multimer is soluble. As used herein, the term “soluble” indicates that the peptide or polypeptide (ie the Fc binding multimer) is not bound to a cellular membrane, and is instead characterised by the absence or functional disruption of all or a substantial part of any transmembrane (ie lipophilic) domain (for example, originating from an FcR from which a Fc binding region of the Fc binding multimer may be derived), so that the soluble Fc binding multimer is devoid of a membrane anchoring function.

The “polypeptide comprising an Fc region or fragment thereof” may be any polypeptide comprising an Fc region or fragment thereof, that is capable of specifically binding with a Fc binding region. Typically, the polypeptide comprising an Fc region or fragment thereof will be an antibody; however, in an embodiment, the polypeptide comprising an Fc region or fragment thereof may be one or more other naturally occurring and/or recombinant and/or synthetic polypeptide(s) comprising an Fc region or fragment thereof. For example, the polypeptide comprising an Fc region or fragment thereof may be a fusion polypeptide comprising an Fc region fused to another peptide or a fragment thereof. Examples of such fusion peptides include a cytokine receptor fusion with an Fc region or a CTLA4 protein fusion with an Fc region.

In an embodiment, the polypeptide comprising an Fc region or fragment thereof is an antibody. The term “antibody” is intended to refer to a particular antibody, or a combination of antibodies, the binding activity of which is of interest; for example, a therapeutic monoclonal antibody or a “cocktail” comprising more than one therapeutic monoclonal antibody (eg a cocktail of monoclonal antibodies targeting different and/or overlapping epitopes of a pathogenic polypeptide (eg a viral protein) or a cancer-cell polypeptide). The antibody could alternatively be an antibody raised in vivo, for example, in response to vaccination or naturally acquired immunity, including a polyclonal antibody (eg as prepared from serum).

In an embodiment, the antibody specifically binds to a specific antigen with its antigen binding domains as will be well understood by a person skilled in the art. The specific antigen may be a particular pathogen, including a bacterium, a virus, a yeast cell, a fungus cell, etc, or be a cellular receptor, a toxin, a soluble peptide, a cell-bound peptide, a biological surface, or another item, again as would be well understood by a person killed in the art. In an embodiment, the specific antigen is a particular epitope from a larger antigenic item. However, in a particular embodiment, the antibody is not an antibody targeted to a pathogenic polypeptide (eg a viral protein). In an embodiment, the antibody is not an antibody targeted to a HIV polypeptide. In an embodiment, the antibody is not an antibody targeted to an influenza peptide.

The “FcR binding activity” of a polypeptide comprising an Fc region or fragment thereof is intended to refer to the ability of the polypeptide (eg an antibody) to specifically bind with a particular Fc binding region and may include, for example, the affinity, avidity and/or overall strength of binding between the specific antigen and the particular Fc binding region. While not wishing to be bound by theory, when cell bound, different FcγR are considered to have different affinity hierarchies for different isotypes of immunoglobulin (Ig) G. This is significant because the binding of differing isotypes of IgG to different FcγR, and the subsequent activation of the FcγR, can induce different types of immunological effector responses as would be understood by those skilled in the art. Moreover, the strength of FcR binding activity can be further influenced by the heavy chain genetic polymorphism (eg allotype), carbohydrate content of the N-linked glycan (eg addition or omission of fucose moieties), and mutations that are naturally occurring or introduced by standard techniques. In addition, the hierarchy or strength of FcR binding activity may be further influenced by the antigen interaction with the Fab component of the antibody to which it is bound (eg the location, repetition and spatial density of the epitope). Accordingly, for an antibody, the binding ability of the antibody to bind to one or more FcγR is of interest in order to promote the production a desired immune response.

There are a number of different types of FcγR, including FcγRI, FcγRIIa, FcγRIIb1, FcγRIIb2, FcγRIIc, FcγRIIIa, and FcγRIIIb. Additionally, there are a number of allelic variants of these receptors. The Fc binding region of the present disclosure may be derived from any of these FcγR. In an embodiment, the Fc binding region is derived from a low affinity FcγR (eg an FcγR having an affinity for monomeric IgG of less than 5×10⁷ M⁻¹).

In an embodiment, the Fc binding region is derived from FcγRIIa which is typically considered to be a low affinity FcγR. FcγRIIa is expressed on cells, especially innate leukocytes, including macrophages, monocytes, professional antigen presenting cells (APC) such as dendritic cells, neutrophils, eosinophils, and mast cells and platelets. Activation of FcγRIIa predominately induces phagocytosis including ADCP, degranulation, mediator release, cytokine production and antigen presentation by the delivery of antigen contained in immune complexes to professional APCs.

The present disclosure encompasses at least two allelic variants (ie polymorphs) of human FcγRIIa; that is, FcγRIIa-His131 and FcγRIIa-Arg131 and, while not wishing to be bound by theory, it is thought that individuals homozygous for FcγRIIa-His131 have a higher binding affinity for IgG1, and more particularly IgG2, compared to individuals homozygous for FcγRIIa-Arg131. Functionally, phagocytes from individuals homozygous for FcγRIIa-His131 have an increased capacity to respond with phagocytosis, degranulation, or cytokine release in response to IgG-opsonised bacteria or erythrocytes, when compared with cells from FcγRIIa-Arg131 homozygous individuals. The binding affinity hierarchy of FcγRIIa-His131 as a cell surface receptor is IgG3>IgG1˜IgG2>>IgG4, whereas the binding affinity hierarchy of FcγRIIa-Arg131 as a cell surface receptor is IgG3>IgG1>IgG2>>IgG4. In an embodiment, the Fc binding region is derived from FcγRIIa-His131. In an embodiment, the Fc binding region is derived from FcγRIIa-Arg131.

The amino acid sequence of the human FcγRIIa isoform 1 precursor, which is also known as FcγRIIa-His131 and is considered to be the human canonical sequence, can be found at UniProt P12318, which is at SEQ ID NO: 1. The standard amino acid numbering for this receptor is based on the convention of numbering the key functional polymorphic residue Arg131, which start numbering at the beginning of the mature peptide rather than the beginning of the leader peptide. For SEQ ID NO: 1, the His131 residue is represented by His167. The N terminus of the mature human FcγRIIa-His131 receptor is conventionally Ala1, which is at amino acid 37 of SEQ ID NO: 1. The ectodomains 1 and 2 that comprise the Fc binding region are conventionally numbered amino acids 1 to 171 (36-206 of SEQ ID NO: 1) and amino acids 172 to 179 (207-214 of SEQ ID NO: 1); which comprise the membrane proximal stalk, which in FcγRIIa links the ectodomains 1 and 2 to the transmembrane sequence. In an embodiment, the Fc binding may comprise amino acids 1 to 171 or amino acids 1 to 179 (as conventionally numbered) or a fragment of the ectodomains 1 and 2 comprising amino acids which includes residues of the ectodomain regions BC (conventionally numbered W110-V116), C′E (conventionally numbered F129-P134) and FG (conventionally numbered G156-Y157) participating in ligand binding.

The amino acid sequence of the human FcγRIIa-Arg131 is shown at SEQ ID NO: 2. Ala1 of the mature peptide is shown as Ala 37 in SEQ ID NO: 2, and based on the convention numbering, the key functional polymorphic residue Arg131 is shown as Arg167 in SEQ ID NO: 2.

Additionally, the present disclosure encompasses at least two allelic variants of macaque FcγRIIa that is, mnFcγRIIa-His131 and mnFcγRIIa-Pro13. Binding studies have demonstrated that the mnFcγRIIa-Pro131 is hypofunctional in binding human IgG1 (Trist et al., 2014). Individual macaques expressing this allelic form of mnFcγRIIa might be expected to develop relatively poor FcγRIIa mediated responses to human IgG therapeutic antibodies in comparison to individuals expressing the mnFcγRIIa-His131. In an embodiment, the Fc binding region is derived from FcγRIIa-Pro131.

The amino acid sequence of macaque FcγRIIa-His131 is provided at SEQ ID NO: 3 (Trist et al., 2014). Ala1 of the mature peptide is shown as Ala 34 in SEQ ID NO: 3, and the conventionally numbered functional polymorphic residue His131 is found at position 164 in SEQ ID NO:3.

In an embodiment, the Fc binding region may comprise amino acids 1 to 171 (as conventionally numbered) or amino acids 1 to 179 (as conventionally numbered) or a fragment of the ectodomains 1 and 2 of macaque FcγRIIa comprising amino acids which includes residues of the ectodomain regions BC (W110-I116), (F129-P134) and FG (G156-Y157) participating in ligand binding.

The amino acid sequence of macaque FcγRIIa-Pro131 is provided at SEQ ID NO: 4 (Trist et al., 2014). Ala1 of the mature peptide is shown as Ala 34 in SEQ ID NO: 4, and the conventionally numbered functional polymorphic residue Pro131 is found at position 164 in SEQ ID NO:4. In an embodiment, the Fc binding region may comprise amino acids 1 to 171 or amino acids 1 to 179 (as conventionally numbered) or a fragment of the ectodomains 1 and 2 of macaque FcγRIIa-Pro131 comprising amino acids which includes residues of the ectodomain regions (as conventionally numbered) BC (W110-I116), C′E (F129-P134) and FG (G156-Y157) participating in ligand binding.

In an embodiment, the Fc binding region is derived from FcγRIIb which is typically considered to be a low affinity FcγR. FcγRIIb is expressed on cells including monocytes, macrophages, B cells and mast cells. Binding of antibody to cellular FcγRIIb inhibits cell activity and some variants inhibit phagocytosis, while other variants may induce phagocytosis. The binding affinity hierarchy of FcγRIIb as a cell surface receptor is IgG3>IgG1>IgG4>IgG2 (Hogarth and Pietersz, 2012).

The amino acid sequence of the human FcγRIIb is provided at SEQ ID NO: 5. Ala1 of the mature peptide is shown as Ala 45 in SEQ ID NO: 5, and based on the convention numbering, the key functional polymorphic residue Arg131 is shown as Arg175 in SEQ ID NO: 5. In an embodiment, the Fc binding region may comprise (conventionally numbered) amino acids 1 to 171 or amino acids 1 to 178 of human FcγRIIb.

The amino acid sequence of the macaque FcγRIIb is shown below as SEQ ID NO: 6 (Trist et al., 2014). Ala1 of the mature peptide is shown as Ala 45 in SEQ ID NO: 6, and based on the convention numbering, the key functional polymorphic residue His131 is shown as His176 in SEQ ID NO: 6. In an embodiment, the Fc binding region may comprise (conventionally numbered) amino acids 1 to 171 or amino acids 1 to 179 of macaque FcγRIIb.

In an embodiment, the Fc binding region is derived from FcγRIII. FcγRIII is generally considered to be a low affinity receptor. There are three isotypes presently known, namely FcγRIIIa, FcγRIIIb and FcγRIIIc. In an embodiment, the Fc binding region is derived from FcγRIIIa. FcγRIIIa may be expressed on cells including NK cells, monocytes and macrophages in certain tissues, and cellular activation through FcγRIIIa binding with antibody may induce effector functions including the induction of antibody-dependent cell-mediated cytotoxicity (ADCC), and cytokine release and mediator release.

The present disclosure encompasses at least two allelic variants of human FcγRIIIa; that is, FcγRIIIa-Val158 and FcγRIIIa-Phe158. The FcγRIIIa-Val158 is considered to be a higher affinity receptor. Reports that therapeutic responses to anti-tumour IgG1 antibodies correlated with patient expression of higher affinity (Val158) alleles of FcγRIIIa has demonstrated the importance of this interaction in the mechanism of action of these antibodies (Weng and Levy, 2003; Musolino et al., 2008). Further, analysis of the glycosylation of the IgG Fc found that the absence of a bisecting fucose in the Asn297 linked carbohydrate greatly increased interaction with FcγRIIIa (Shinkawa et al., 2003; Ferrara et al., 2006; Ferrara et al., 2011). Therapeutic antibodies that lack this fucose were shown to have improved receptor binding and target killing in pre-clinical studies (Iida et al., 2006; Herter et al., 2013) and improved responses in the clinic (Cameron and McCormack, 2014; Illidge et al., 2014; Rogers and Jones, 2014). The binding affinity hierarchy of both the FcγRIIIa-Val158 and FcγRIIIa-Phe158 variants have as cell surface receptors is IgG3>IgG1>>IgG4 IgG2 (Hogarth and Pietersz, 2012; Bruhns et al., 2009). In an embodiment, the Fc binding region is derived from FcγRIIIa-Phe158.

The amino acid sequence of the human FcγRIIIa-Val158 (SEQ ID NO: 7), also known as the low affinity immunoglobulin gamma Fc region receptor IIIa, is provided at SEQ ID No: 7. Arg1 of the mature peptide is shown as Arg19 in SEQ ID NO: 7, and based on the convention numbering, the key functional polymorphic residue Val158 is shown as Val167 in SEQ ID NO: 7. In an embodiment, the Fc binding region of FcγRIIIa-Val158 may comprise (conventionally numbered) amino acids 1-174.

The amino acid sequence of the human FcγRIIIa-Phe158 (SEQ ID NO: 8), also known as the low affinity immunoglobulin gamma Fc region receptor IIIc, is provided at SEQ ID NO: 8. Arg1 of the mature peptide is shown as Arg55 in SEQ ID NO: 8, and based on the convention numbering, the key functional polymorphic residue Phe158 is shown as Phe212 in SEQ ID NO: 8. In an embodiment, the Fc binding region of FcγRIIIa-Phe58 may comprise (conventionally numbered) amino acids 1-174.

In an embodiment, the Fc binding multimer comprises at least two Fc binding regions are derived from an Fcγreceptor (FcγR). In an embodiment, the Fc binding multimer comprises at least two Fc binding regions derived from an FcγR selected from the group consisting of FcγRI, FcγRIIa, FcγRIIb1, FcγRIIb2, FcγRIIIa, FcγRIIIb and FcγRIIIc. In an embodiment, the Fc binding multimer comprises at least two Fc binding regions derived from an FcγR selected from the group consisting of FcγRIIa, FcγRIIb, FcγRIIIa and FcγRIIIb. In an embodiment, the Fc binding regions are derived from a FcγR selected from the group consisting of FcγRIIa-His131, FcγRIIa-Arg131, FcγRIIa-Pro131, FcγRIIb, FcγRIIIa-Val158, and FcγRIIIa-Phe158. In an embodiment, the Fc binding regions are derived from an amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8 as disclosed herein. In an embodiment, the Fc binding regions consist of a Fc binding fragment of an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

FcγRIIa is the most widespread Fc receptor being present on all innate leucocytes and platelets but is absent from lymphocytes. Usually FcγRIIa is activating type FcγR that when clustered by oligo valent immune complexes, signals through the immunoreceptor tyrosine activation motif (ITAM) pathway. As a consequence of its broad cellular distribution, FcγRIIa is involved in activation of a range of effector cell functions including phagocytosis by professional phagocytes such as macrophages (reviewed in Hogarth and Pietersz, 2012; and Immunological Reviews volume 268 in its entirety), antigen presentation particularly by dendritic cells and long-term vaccine-like immunity following monoclonal antibody therapy (Dilillo et al., 2015), granulocyte activation leading to tissue destruction. Its expression on individual cell types may be influenced by environmental factors to which the cells are exposed for example a site of active inflammation, a cancer mass, infection or a site of resolving inflammation. Whilst normally recognised as an activating receptor, in situations of low receptor occupancy for example exposure to high concentrations of monomeric Ig, FcγRIIa can deliver inhibitory signals via the Immunoreceptor tyrosine-based activation motif (ITAM; ITAMi).

Genetic polymorphism of the FcγRIIa gene can result in receptor proteins of different amino acid sequence which have altered receptor function in humans and nonhuman primates. Such altered function is includes alterations to the interaction with antibody Fc and or alterations in signalling. FcγRIIa-His131 and FcγRIIa-Arg131 are polymorphic forms of human FcγRIIa. They differ in the amino acid at position 131 of the ectodomains. Amino acids at position 131 can form part of the surface of the FcγRIIa that is in contact with the antibody Fc portion and so this polymorphism influences the strength and specificity of interaction with human immunoglobulins such that human IgG2 binds poorly to the FcγRIIa-Arg131 but strongly to FcγRIIa-His131. Differences in the specificity and strength of binding result in altered signalling and cellular effector responses and clinical outcomes through FcγRIIa induced by IgG, particularly IgG2. Thus, for example, the efficiency of responses to bacterial infection can be affected by allelic variants of FcγRIIa in patients (reviewed in Hogarth and Pietersz, 2012). Thus target antibodies that have a high binding affinity for FcγRIIa-His131 may have enhanced capacity to induce more potent effector functions (eg enhanced phagocytosis or improved delivery of immune complexes for better antigen presentation).

FcγRIIa-Pro131 is a polymorphic form of FcγRIIa found in macaque nemestrina. When expressed on the cell surface it shows profound impairment of binding of all human IgG classes (Trist et al., 2014) and so would be expected to show consequence of impairment of signalling and cell effector functions.

FcγRIIb is expressed on many, though not all, leucocytes in humans and other important mammalian species. It is an inhibitory type Fc receptor that modulates the activating signals of ITAM signalling pathways of the B-cell antigen receptor and activating type Fc receptors. The inhibitory signalling of FcγRIIb occurs via an immunoreceptor tyrosine inhibitory motif (ITIM) pathway. The inhibitory action of FcγRIIb on effector functions requires that it is co-aggregated with receptors that signal through the ITAM pathway. Thus, FcγRIIb on B lymphocytes, where it is the only FcγR, acts by co-engagement with the antigen receptor to modulate B-cell function. On cells of the innate immune system, co-engagement of FcγRIIb with activating receptors such as FcγRIIIa on macrophages decreases phagocytosis or co-engagement with the high affinity IgE receptor FcεRI on basophils decreases degranulation and mediator release. Thus, target antibodies that have a high binding affinity for FcγRIIb may have enhanced inhibition of cell functions (eg greater inhibition of B cell antigen receptor activation leading to further reduction in antibody production or in innate immune cells, greater reduction of macrophage phagocytosis or mediator release by, for example, basophils or other myeloid cells).

FcγRIIIa is a multi-subunit Fc receptor activating type FcγR. The Fc binding polypeptide chain of the receptor complex is non-covalently associated with a low molecular weight dimer wherein each chain of the dimer contains an ITAM. The dimer can be homo- or hetero-dimeric, being formed from chains derived from the so-called common FcRγ-chain or the CD3ζ-chain. It is expressed on some lymphocytes such as γδ-T cells and NK cells where it is responsible for ADCC which allows the cytotoxic killing of cells such as virus-infected cells or cancer cells that are opsonised with antibody; for example, vaccine-induced antibodies or therapeutic monoclonal antibodies. Such cell killing can also include killing of normal cells opsonised with auto-antibodies. FcγRIIIa is also expressed on macrophages and is important in the phagocytic uptake of opsonised particles for example virus-infected cells or cancer cells. Such phagocytosis can include normal cells opsonised with auto-antibodies.

Genetic polymorphism of the FcγRIIIa gene can result in receptor proteins of different amino acid sequence which have altered receptor function in humans and nonhuman primates. Such altered function includes alterations to interactions with antibody Fc and or alterations in signalling. FcγRIIIa-Val158 and FcγRIIIa-Phe158 are polymorphic forms of human FcγRIIIa. They differ in the amino acid at position 158 of the ectodomains. Amino acids at position 158 can form part of the surface of the FcγRIIIa that is in contact with the antibody Fc portion. This polymorphism influences the strength and specificity of interaction with human immunoglobulins such that human IgG binds strongly to the FcγRIIIa-Val158 but poorly to FcγRIIIa-Phe158. Differences in the specificity and strength of binding result in altered signalling and cellular effector responses and clinical outcomes through FcγRIIIa. Thus, for example, the efficiency of ADCC cell killing responses is significantly greater by killer cells expressing FcγRIIIa-Val 158 compared with killer cells expressing FcγRIIIa-Phe158 therapeutic monoclonal antibodies in patients (reviewed in Hogarth and Pietersz, 2012).

Thus target antibodies that have a highly binding affinity for FcγRIIIa-Val158 or FcγRIIIa-Phe158 may have enhanced capacity to induce more potent effector functions for example enhanced ADCC by NK cells or improved phagocytosis by macrophages.

In an embodiment, the Fc binding multimer may consist of an Fc binding peptide that is a fusion polypeptide comprising at least two Fc binding regions that are genetically linked. The fusion polypeptide may be encoded by a nucleic acid molecule coding for a genetically linked fusion of two or more Fc binding regions. In embodiments encompassed by the present disclosure, the Fc binding multimer may consist of a fusion polypeptide comprising, for example, two, three, four, five, six, seven, eight, nine or ten Fc binding regions that are genetically linked. In an embodiment, the Fc binding multimer may be an Fc binding dimer consisting of a fusion polypeptide comprising two Fc binding regions. In an embodiment, the Fc binding multimer may be an Fc binding trimer consisting of a fusion polypeptide comprising three Fc binding regions. In an embodiment, the Fc binding multimer may be an Fc binding tetramer consisting of a fusion polypeptide comprising four Fc binding regions.

In an embodiment, the Fc binding regions of an Fc binding multimer fusion protein may be joined by a linker peptide. A number of different linker peptides may be suitable for the multimer of the present disclosure. For example, the linker peptide may be the amino acid sequence VPSMGSSSPVA (SEQ ID NO: 23). In alternative embodiments, the peptide linker may comprise other suitable known peptide linkers of lengths between approximately 2 and 50 amino acids, such as can be found in peptide linker databases such as those linkers known as PLrigid, 2aa GS linker, baa [GS]x linker, 10aa[GS]x linker, 10 aa flexible protein domain linker, 8 aa protein domain linker, Flexible linker 2x (GGGS), flexible linker 2x (GGGGS), 13 amino acids linker [GGGS GGGGS GGGS], 10 aa linker, Split fluorophore linker (Freiburg standard), 15 aa flexible glycine-serine protein domain linker (Freiburg standard), Short Linker (Gly-Gly-Ser-Gly), Middle Linker (Gly-Gly-Ser-Gly)x2, Long Linker (Gly-Gly-Ser-Gly)x3, GSAT Linker, SEG, SEG-Linker, GSAT-Linker, Z-EGFR-1907_Short-Linker, Z-EGFR-1907_Middle-Linker, Z-EGFR-1907_Long-Linker, Z-EGFR-1907_SEG-Linker, (Gly4Ser)3 Flexible Peptide Linker, Short Fusion Protein Linker: GGSG with standard 25 prefix/suffix, Long 10AA Fusion Protein Linker with Standard 25 Prefix/Suffix, Medium 6AA Fusion Protein Linker (GGSGGS with Standard 25 Prefix/Suffix), etc.

In an embodiment, the Fc binding peptides of the present invention further comprise an N-terminal leading sequence or signal sequence, as would be understood by a person skilled in the art. In an embodiment, the Fc binding multimer has a native signal sequence derived from a FcγR such as FcγRIIa or from an immunoglobulin such as a human IgG. However, a person skilled in the art will appreciate that a wide range of N-terminal leader sequences may be suitable.

In an embodiment, the Fc binding peptides disclosed herein may further comprise sequences that may assist with peptide detection and/or peptide purification, such as a His tag. However, a person skilled in the art will appreciate that a great many detection and/or purification tags could be used in the Fc binding peptides of the present invention.

In an embodiment, the Fc binding peptides disclosed herein may further comprise sequences that may assist with detection and/or oligomerisation. For example, the Fc binding peptides of the present invention may comprise a BirA ligase target sequence. The Bir A ligase target sequence consists of an amino acid motif that is recognised by and specifically bound by a Bir A enzyme, which mediates biotinylation of the Fc binding peptides. The biotin on the peptides can be used to detect the presence of the Fc binding peptide (eg by binding with streptavidin or avidin that is bound to a suitable detection tag). In an alternative or additional embodiment, the biotin on the Fc binding peptide can be used to oligomerise two or more biotin-labelled Fc binding peptides together to form an oligomerised Fc binding multimer as described herein. However, a person skilled in the art will appreciate that other detection or oligomerisation sequences may be present in the Fc binding peptides and used to oligomerise Fc binding peptides together, and/or to detect the presence of the Fc binding peptide as described herein.

In an embodiment, the Fc binding peptides disclosed herein may further comprise other useful sequence features, such as a cleavage sequence, and/or other binding domains, etc, as would be understood by a person skilled in the art.

In an embodiment, the Fc binding peptides may comprise an amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

In an embodiment, the Fc binding multimer consists of an Fc binding peptide.

In an embodiment, the Fc binding multimer consists of at least two distinct Fc binding peptides each comprising at least one Fc binding region, wherein the at least two distinct Fc binding peptides have been oligomerised covalently or non-covalently to form an oligomerised Fc binding multimer. In an embodiment, the Fc binding multimer consists of at least two polypeptides, each comprising a monomer of the Fc binding region, that have been oligomerised non-covalently to form an oligomerised Fc binding dimer. In an embodiment, the Fc binding multimer consists of at least two fusion polypeptides, each comprising a dimer of Fc binding regions, that have been oligomerised non-covalently to form an oligomerised Fc binding tetramer. However, it is envisioned that fusion peptides can each comprise a trimer of Fc binding regions, a tetramer of Fc binding regions, or more copies of the Fc binding region. In an embodiment, these fusion peptides can be oligomerised non-covalently to form an oligomerised Fc binding multimer. Moreover, two, three, four, or more of these fusion polypeptides, each containing one, two, three, four, or more Fc binding regions can be oligomerised together to form the Fc binding multimers. In an embodiment, the Fc binding multimer consists of at least two monomer Fc binding regions that have been oligomerised non-covalently. In an embodiment, the Fc binding multimer consists of at least two dimer Fc binding regions that have been oligomerised non-covalently. In an embodiment, the Fc binding multimer consists of at least two trimer Fc binding regions that have been oligomerised non-covalently. In an embodiment, the Fc binding multimer consists of at least two tetramer Fc binding regions that have been oligomerised non-covalently. In an embodiment, the at least two monomer, dimer, trimer or tetramer Fc binding regions are three monomer, dimer, trimer or tetramer Fc binding regions. In an embodiment, the at least two monomer, dimer, trimer or tetramer Fc binding regions are four monomer, dimer, trimer or tetramer Fc binding regions. A person skilled in the art will also appreciate that if four copies of a tetramer of Fc binding regions are oligomerised together, the resulting Fc binding multimer would have 16 copies of the Fc binding region, etc. In an embodiment, the peptides oligomerised together may have different numbers of Fc binding regions, such that the produced oligomerised Fc binding multimer may have a range of numbers of Fc binding regions.

Accordingly, in an embodiment, the Fc binding peptides may be oligomerised with the use of a specific binding pair, such as biotin and streptavidin, or biotin and avidin, although the present disclosure also encompasses any other suitable ligand binding pairs. The Fc binding peptides can, for example, be bound with one partner of a specific binding pair, and then the Fc binding peptides can be oligomerised into a multimer upon the addition of the second partner of the specific binding pair. In an embodiment, Fc binding regions can be biotinylated, and then oligomerised in the presence of streptavidin or avidin, both specific binding partners for biotin. The Fc binding regions can be biotinylated using standard methods, for example, by chemically biotinylating the peptide in vitro. In an embodiment, the Fc binding region is modified to be fused to a biotinylation sequence such as the AviTag sequence GLNDIFEAQKIEWHE (SEQ ID NO: 9), which can be biotinylated by the BirA enzyme either within the cellular expression system producing the Fc binding regions or in vitro. Both streptavidin and avidin are tetramers, and each subunit of the tetramers binds biotin with equal affinity. Therefore, the addition of streptavidin or avidin to biotinylated monomer Fc binding regions may result in the oligomerisation of Fc binding multimer complexes with two, three or four Fc binding peptides (ie Fc binding tetramers). Accordingly, in an embodiment, the Fc binding multimer consists of at least two Fc binding peptides each comprising an at least one Fc binding region, wherein each Fc binding peptide is bound to a first partner of a specific binding pair such that when a second partner of the specific binding pair is present, at least two of the first partners each bind with the second partner of the specific binding pair to form the Fc binding multimer. For example, the Fc binding multimer consists of at least two Fc binding peptides each comprising an at least one Fc binding region, wherein each Fc binding peptide is bound to biotin such that when streptavidin or avidin is present, at least two biotin molecules each bind with the same streptavidin or avidin molecule to form the Fc binding multimer.

Other specific binding pairs are encompassed by the present disclosure, for example, a secondary antibody may specifically bind via its antigen-specific Fab domain to a primary antibody, a tertiary antibody may specifically bind via its antigen-specific Fab domain to a secondary antibody, etc, and other receptor-ligand pairs may be suitable for use. In some embodiments, a primary binding pair may be used to form the Fc binding multimer, and a secondary or tertiary binding pair may also be used, for example in surface binding the interaction between the Fc binding region and an antibody, or alternatively in detecting or identifying the binding between the Fc binding multimer and the antibody. In an embodiment, the Fc binding multimer may be oligomerised using other methods, for example, chemical cross-linking using techniques known to a person skilled in the art.

The Fc binding peptides described herein, in their monomer or genetic fusion forms can be produced using expression systems known to those skilled in the art, such as bacterial expression systems, eukaryotic cells expression systems, insect cell expression systems, yeast expression systems or they can be synthetically produced.

The cellular effector response induced by IgG antibodies is mediated by a combination of IgG subclass specificity and affinity for FcγR, and the functional nature of the type of cell expressing one or more FcγR, which are the receptors for IgG. When antibodies bind to the FcγR, the binding subsequently induces clustering or cross-linking of the FcR, and induces cell effector responses. These responses typically involve activation of effector cells such as a phagocytes or killer cell but the responses can also involve a modulation of activation through an inhibitory mechanism. It is desirable to evaluate the binding activity of antibodies to different FcR, as there are differences in the specificity of the human IgG subclasses for the different FcγR and differences in the cell distribution of the different FcγR types.

Activating receptors include FcγRIIa, FcγRIIIa which activate effector cells through the ITAM (Immunoreceptor tyrosine activation motif); whereas the archetypal or canonical inhibitory functions are mediated by antibody-induced clustering of FcγRIIb through the ITIM (Immunoreceptor tyrosine inhibitory motif) pathway.

The four human IgG subclasses (IgG1, IgG2, IgG3, IgG4) show some similarity in the specificity of interaction with Fc receptors but also show key differences. For example, IgG3 binds to all human FcγR whereas IgG4 binds only to FcγRI and FcγRIIb.

The human FcγR also show differences in their distribution on different effector leucocytes. For example, FcγRIIa is the most widespread FcγR being present on all lineages of innate leucocytes (monocytes, dendritic cells and macrophages, mast cells and basophils, neutrophils and platelets but not present on lymphocyte lineages i.e. T and B lymphocytes and NK cells;

NK cells express FcγRIIIa, and ADCC is mediated through this receptor on the cells. Thus antibodies that have preferential or even exclusive binding to FcγRIIIa have improved therapeutic action where ADCC is required to kill a target cell. Clustering the same receptor on a different cell may facilitate a different response. Macrophages also express FcγRIIIa and phagocytic uptake (ie ADCP) of opsonised particles and/or cells is mediated by this receptor. Antibodies that have preferential or even selective binding to FcγRIIIa may have improved therapeutic action where phagocytosis is required to eliminate a target.

Neutrophils release mediators and phagocytosis is induced in response to FcγRIIa activation, and therefore, therefore, FcγRIIa binding antibodies may induce neutrophil function or mediator release. Such antibodies include antibodies of the human IgG2 subclass, which is known to bind only to FcγRIIa and only to the allelic variant containing histidine at position 131 (FcγRIIa H 131). Antibodies that can be engineered to selectively engage FcγRIIa will also mediate such effector responses.

The inhibitory Fc receptor FcγRIIb is a powerful modulator of cell function when co-clustered with an activating-type receptor that uses the ITAM pathway, such as the B-cell antigen receptor or an activating Fc receptor. Antibodies that co-engage the FcγRIIb on B cells with the antigen receptor complex can reduce B cell activation and antibody production. Antibodies that co-cluster this receptor with an activating Fc receptor on innate effector cells such as basophils can reduce pro-inflammatory responses of the basophil induced by immune complexes or allergen and IgE. IgG subclasses like IgG4 or engineered antibodies that to selectively engage this receptor can produce potent inhibitory responses in inflammatory cells.

Additionally, for individuals known to have a particular FcR allelic variant, it is advantageous to identify antibodies that will effectively bind to the variant. Additionally, it may be of interest to determine the Fc binding activities of antibodies raised in vivo, for example, in response to vaccination or naturally acquired immunity, including a polyclonal antibody (eg as prepared from serum).

Accordingly, the Fc binding multimers of the present disclosure can be used in a variety of assays and methods to assess the Fc binding characteristics of Fc containing peptides, such as antibodies. In an aspect, the present disclosure relates to an in vitro Fc binding assay that is capable of measuring the Fc binding of a polypeptide comprising an Fc region or fragment thereof (eg an antibody) using an Fc binding multimer disclosed herein. In an aspect, the present disclosure relates to an in vitro method of determining Fc binding activity of a polypeptide comprising an Fc region or fragment thereof (eg an antibody) using an Fc binding multimer disclosed herein.

Accordingly, in an aspect, the present disclosure relates to a method of assessing in vitro the FcR binding activity of an antibody comprising an Fc region or fragment thereof, the method comprising the following steps:
a. providing an at least one Fc binding multimer comprising at least two Fc binding regions;
b. incubating the antibody with the at least one Fc binding multimer under conditions suitable to permit specific binding of the Fc binding regions to a Fc region or fragment thereof and
c. identifying the magnitude of FcR binding activity of the antibody with the Fe binding multimer using a label agent;

wherein the magnitude of FcR binding activity identified in step (c) provides an assessment of the FcR binding activity of the antibody.

Further, in another aspect, the present disclosure relates to an assay to assess in vitro the FcR binding activity of an antibody comprising an Fe region or fragment thereof, the method comprising the following steps:
a. providing an at least one Fc binding multimer comprising at least two Fc binding regions;
b. incubating the antibody with the at least one Fc binding multimer under conditions suitable to permit specific binding of the Fe binding regions to a Fc region or fragment thereof; and
c. identifying the magnitude of FcR binding activity of the antibody with the Fc binding multimer using a label agent;

wherein the magnitude of FcR binding activity identified in step (c) provides an assessment of the FcR binding activity of the antibody.

Further, in another aspect, the present disclosure relates to a method of selecting an at least one antibody or fragment thereof having an Fc region with a desired FcR binding activity from a panel of antibodies or fragments thereof, the method comprising
a. providing an Fc binding multimer comprising at least two Fc binding regions, wherein the Fc binding regions are derived from a pre-selected Fc receptor (FcR);
b. separately incubating each of the panel of antibodies or fragments thereof with the Fc binding multimer under conditions suitable to permit specific binding of an antibody with a Fc region having the preferred FcR binding activity with the Fc binding multimer;
c. detecting magnitude of binding of each of the panel of antibodies to the Fc binding multimer; and
d. selecting from the panel of antibodies an antibody having a high magnitude of binding with the Fc multimer;
   wherein the selected antibody has the desired FcR binding activity. In an embodiment, the desired FcR binding activity may be a pre-selected FcR binding activity, a preferred FcR binding activity, a predetermined binding activity, an optimal FcR binding activity, etc.

In an embodiment of the method or assay, the magnitude of FcR binding activity identified is qualitatively measured. In an embodiment, the magnitude of FcR binding activity identified is qualitatively measured.

In an embodiment, the methods or assays of the present invention can be performed on any suitable surface known to a person skilled in the art, for example, on a plate (eg an ELISA plate, or other type of tissue culture plate or other plate), on beads (eg magnetic beads, fluorescent beads, polystyrene beads, etc), fibres, membranes, bio-sensors, etc. Accordingly, in an embodiment, the methods or assays of the present invention can be immobilised to a surface. In an embodiment, step (b) further comprises immobilising the antibody by binding with an immobilised specific antigen or an immobilised immunoglobulin or fragment thereof which specifically binds with the antibody, prior to incubating the antibody with the at least one Fc binding multimer. In this embodiment, a binding partner of the antibody is bound to the surface, by incubating the antibody binding partner on the surface under conditions that promote binding of the binding partner to the surface. The binding partner may include a specific target antigen, a super-antigen, a mitogen, or an immunoglobulin, etc. The antibody of interest may then be bound to the immobilised binding partner under conditions that promote binding of the binding partner to the surface. The Fc binding multimer may be applied to the antibody under conditions that mediate specific binding of the Fc binding multimer to the Fc portion of the antibody. In an embodiment, binding of the Fc binding multimer may be detected using a method described herein.

In an embodiment, the methods or assays of the present invention can be performed on cellular surfaces, and detected using techniques known to a person skilled in the art, such as by flow cytometry or fluorescent activated cell sorting (FACS) analysis. Accordingly, in an embodiment, step (b) further comprises incubating the antibody with a cell bound specific antigen, or a cell-fragment-bound specific antigen to bind the antibody to the cell or cell fragment, prior to incubating the antibody with the at least one Fc binding multimer. In this embodiment, the specific antigen is a cellular receptor or cell bound peptide under conditions that promote the specific binding of the antibody to an antigen. The Fc binding multimer may be applied to the antibody under conditions that mediate specific binding of the Fc binding multimer to the Fc portion of the antibody. In an embodiment, binding of the Fc binding multimer may be detected using a method described herein.

In an embodiment, the methods or assays of the present invention may occur in solution.

The Fc binding activity of an antibody may be affected by antibody subclass, species differences, antibody density, receptor allotype, antibody presentation/topology, and/or other features such as Fc fucosylation. In an embodiment, these methods and assays provide a means to screen target antibodies that are of interest (for example, due to their antigen specificity) to determine their FcR binding activity, which may provide valuable insight as to the strength of Fc binding to a particular FcR and/or type of effector function the antibody may induce in vivo. In an embodiment, the Fc binding activity of an antibody may be assessed by comparison with the Fc binding activities of control antibodies with known Fc binding activities. Accordingly, in an embodiment, the method or assay further comprises comparing the magnitude of FcR binding activity between the Fc binding multimer and the antibody with the magnitude of binding between the Fc binding multimer and one or more control antibodies.

In an embodiment, an increased magnitude of FcR binding activity of the antibody compared to a control antibody demonstrates that the Fc region of the antibody has stronger binding activity for the Fc binding region than the control antibody, a decreased magnitude of binding of the antibody compared to a control antibody demonstrates that the antibody has weaker binding activity for the Fc binding region than the control antibody, or unchanged or similar magnitude of binding of the Fc region of an antibody compared to a control antibody demonstrates that the antibody has the same or similar binding activity for the Fc binding region as the control antibody.

In an embodiment, the method or assay further comprises comparing the magnitude of FcR binding activity between the antibody and a plurality of different Fc binding multimers to identify which Fc receptors (FcRs) the antibody preferentially binds with, and wherein the identifying of preferential binding to a FcR indicates whether the antibody will in use induce a preferred, desired or optimal immune response outcome. This may allow for the selection of an antibody that induces a preferred immune effector response, for example, selected from the group consisting of ADCC, ADCP, degranulation, mediator release, cytokine production and antigen presentation, etc. The preferred immune effector response may vary depending upon the planned use and/or antigen specificity of the antibody.

In an embodiment, an antibody having a preferred Fc binding activity may be selected from a panel of different antibodies specific for the same target antigen. This may allow for the selection of an antibody that induces a preferred immune effector response, for example, ADCC, ADCP, degranulation, mediator release, cytokine production and antigen presentation, etc. Accordingly, in an embodiment, the method or assay further comprises comparing the magnitude of FcR binding activity between the Fc binding multimer and a panel of different antibodies each specific for a same specific antigen to identify an antibody that preferentially binds to a Fc binding multimer comprising at least two Fc binding regions derived from a selected FcR. The selected FcR may be determined on the basis of the type or magnitude of the preferred immune effector response desired to be induced by the antibody.

The preferred effector immune response may change dependent upon the specific antigen and the intended purpose of the antibody. In an embodiment, it may be desirable that the immune effector function induced by an administered antibody is minimised, for example, for certain neutralising antibodies or antibodies that are used to treat autoimmune diseases, etc. However, in an alternative embodiment, it may be preferable that the antibody induce a strong effector immune response, for example, in some cancers and infectious diseases including viral diseases. In an embodiment, the induced immune response is selected from ADCC, ADCP, degranulation, mediator release, cytokine production antigen presentation, complement activation and platelet activation.

In an embodiment, the Fc binding multimers of the present disclosure can be used in methods or assays that determine the ability of a specific antigen to opsonise a target, for example a target cell.

In an embodiment, the Fc binding multimers of the present disclosure can be used in methods or assays that determine the ability of antibody to induce cellular activation of immune cells or to induce inhibition of immune cells through FcγRIIb or non-canonical inhibitory signalling through the activating FcγR (eg ITAMi). The immune cells may include B lymphocytes, NK cells, macrophages, neutrophils, eosinophils and monocytes.

In an embodiment, step (b) of the method or assay further comprises incubating the antibody with an antigenic target or target cell under conditions suitable to permit opsonisation of the antigenic target or target cell with the antibody, and then incubating the antibody with the at least one Fc binding multimer; wherein the FcR binding activity of the antibody indicates whether opsonisation of the antigenic target or target cell with the antibody is likely to induce a cellular induced response or lack thereof. In an embodiment, the cellular induced response is selected from the group consisting of antibody dependent cellular cytoxicity (ADCC), antibody dependent cellular phagocytosis degranulation, mediator release, cytokine production and antigen presentation.

As described herein, the Fc binding multimers can be used to map the proximity of epitopes within the same antigen that different antibodies specifically bind to. Accordingly, in an embodiment of the method or assay, the FcR binding activity of at least two target antibodies are simultaneously assessed to map the proximity of at least two antibodies when bound to a target antigen.

In an embodiment, the Fc binding multimers of the present disclosure can be used in methods or assays that assess the immunity of an individual following vaccination or exposure to a pathogen.

Similarly, in a further aspect, the present disclosure relates to a method of assessing in vitro the FcR binding activity of an antibody comprising an Fc region or fragment thereof, the method comprising:
a. incubating an antibody comprising an Fc region or fragment thereof with at least one Fc receptor (FcR) multimer that binds with the Fc region of the antibody under conditions suitable to permit specific binding of the Fc region of the antibody with the Fc binding multimer; and
b. quantitatively identifying the magnitude of binding of the antibody and Fc binding multimer with a label agent;
   such that the magnitude of binding of the Fc binding multimer with the antibody assesses the FcR binding activity of the antibody.

Detection of binding of the Fc multimer to the Fc region of the antibody can be performed using standard techniques known to those skilled in the art, for example, using a “label agent”. As used herein, the term “label agent” is intended to refer to molecule such as an enzyme tag (eg horseradish peroxidase or alkaline phosphatase), a fluorescent tag, a radioisotope tag or electrochemiluminescent tag, etc, retained by the binding of the Fc region of an antibody with the Fc binding multimer of the present disclosure, and moreover, which produces a “staining” that is detectable using methods well known to those skilled in the art such as ELISA protocols, FACS analysis, electrochemiluminescence analysis, radioisotope analysis, etc. Accordingly, when the Fc binding multimer binds with an antibody, the label agent is immobilised and detected using methods well known to those skilled in the art. In an example, the antibody may be immobilised on a surface (such as a cellular surface or an in vitro surface such as an assay plate). The Fc binding multimer may be non-covalently bound to a first partner of a specific binding pair (eg biotin), such that when the Fc binding multimer binds with the Fc region of the antibody, the multimer is immobilised to the surface. The label agent may be non-covalently bound to a second partner of a specific binding pair (eg streptavidin), which may in turn bind to the immobilised Fc binding multimer. The label agent can then be detected using standard ELISA, FACS, electrochemiluminescence analysis, radioisotope analysis etc protocols.

The label agent enables quantitatively identification of the magnitude of binding of the Fc binding multimer with the Fc region of the antibody. For example, the higher the level of binding activity between the Fc binding multimer and the antibody, the higher the level of the labelling agent that may be immobilised. This in turn can mediate the intensity of the staining caused by the label agent. In an embodiment, the staining can be qualitatively assessed, for example by considering whether the staining in a reaction of interest is more or less intense than the staining in a control reaction. However, the staining can alternatively be quantitatively assessed by comparing the intensity of staining with the intensity of staining of known standards. In an embodiment, the Fc binding assay further comprises comparing the magnitude of binding between the Fc binding multimer and the antibody with the magnitude of binding between the Fc binding multimer and one or more control antibodies.

The magnitude of the binding activity of the antibody may be assessed in comparison to the magnitude of control antibodies, the binding activity of which is known for the same Fc binding multimer. In an embodiment, an increased magnitude of binding of the antibody compared to the control antibody demonstrates that the Fc region of the antibody has stronger binding activity for the FcR than the control antibody. In an embodiment, a decreased magnitude of binding of the antibody compared to the control antibody demonstrates that the antibody has weaker binding activity for the FcR than the control antibody. In an embodiment, an unchanged magnitude of binding of the Fc portion or fragment thereof of an antibody compared to the control antibody demonstrates that the antibody has the same or similar binding activity for the FcR as the control antibody.

A panel of target antibodies, for example, directed against a particular antigen of interest, can be screened individually or in combination using the multimer, assay and method of the present disclosure, to identify which of the target antibodies, or combinations of antibodies together, have FcR binding activity that is able to induce the desired immune system effector function. In an embodiment, it may be advantageous to screen a panel of target antibodies and identify a number of antibodies that are able to induce different effector functions, or that target different FcγR allelic variants, to be able to adapt or personalise therapy for a particular individual. Accordingly, in an embodiment, the magnitude of binding activity between the Fc binding multimer and the antibody is compared with the magnitude of binding activity between different Fc binding multimer(s) and the antibody.

The antibody (including, for example, a combination of antibodies) may be detected in the form of immune complexes (ICs), that is, bound with the specific antigen for the antibody, including complexes with multivalent binding of the target antigen. Alternatively, the antibody may be in the form of surrogate immune complexes, for example, immune complexes formed by the multivalent binding of a secondary antibody that specifically recognises with its Fab binding regions and binds to the target antigen. Such secondary antibodies may be specific for the species or isotype of the antibody, for example, the secondary antibody may be an anti-human IgG2 immunoglobulin, etc, as would be understood by those skilled in the art.

The assay and method described herein may utilise a number of sequential binding steps. In an embodiment, the surfaces (including cellular surfaces) are washed well several times with an appropriate buffer (eg PBS, TBS, optionally with detergents such as Tween 20, etc) between each binding step, and the binding step may be conducted in the presence of a suitable blocking buffer (eg PBS-BSA, TBS-BSA, optionally in the presence of EDTA, Tween 20 etc) to reduce any non-specific binding.

The antibody (including, for example, a combination of antibodies), may be immobilised to a surface (such as an in vitro surface such as an assay plate surface such as an ELISA plate, or alternatively a cellular surface) by direct adsorption or due to the presence of the specific antigen for the antibody on the surface. Accordingly, in an embodiment of the assay and method disclosed herein, the antibody binds with cell bound antigen on the cellular surface. The cell may be a prokaryotic cell, a eukaryotic cell, a mammalian cell, an insect cell, a parasite, a yeast cell, etc. In an embodiment, the antibody binds with cell fragment bound antigen (eg on a platelet, or membrane bound cell fragment derived from a cell). In an embodiment, the antibody binds with specific antigen on an in vitro surface. The antibody may be immobilised to the surface through specific binding with the antigen through its antigen binding (Fab) domains. Alternatively, a secondary antibody (or F(ab′)₂ or Fab antigen binding domains thereof) that is specific for the antibody (that is, for example, a secondary anti-immunoglobulin antibody that specifically binds with, for example, the species and/or isotype of the antibody (such as a human anti-IgG1 antibody, etc)), may be immobilised or bound on an in vitro surface such as a plate surface, and the antibody may be immobilised on the surface through the specific binding of the antigen binding (Fab) domains of the secondary antibody. The Fc binding multimer may then be applied, and the Fc binding multimer may be immobilised to the surface. The label agent may then be applied, which may bind to the immobilised Fc binding multimer via the non-covalent interactions of a specific binding pair, and the label agent may then be qualitatively or quantitatively detected.

In an embodiment, the specific antigen is immobilised by binding with an immobilised specific antigen or immobilised immunoglobulin able to specifically bind with the target antigen. The magnitude of binding can be assessed as described elsewhere herein. However, the present disclosure encompasses a method or assay wherein the Fc binding multimer is immobilised to an in vitro surface, and the antibody is applied, and binding is detected as described elsewhere in, for example, using a label agent non-covalently bound to a secondary antibody specific for the target antigen.

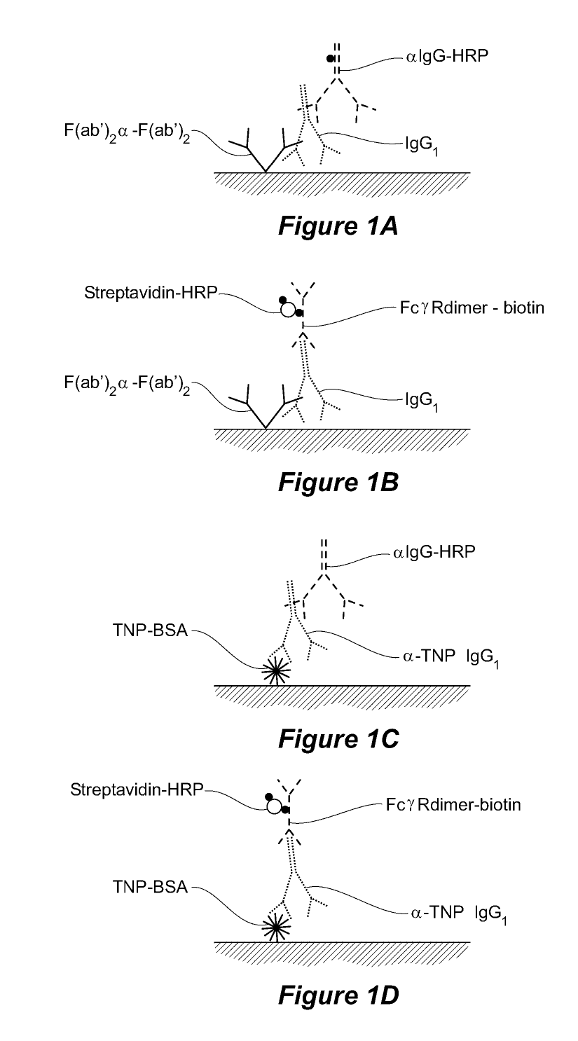

Some embodiments of the assay and method of the present disclosure are shown in FIG. 1.

In an embodiment, the use, assay or method disclosed herein may occur in solution.

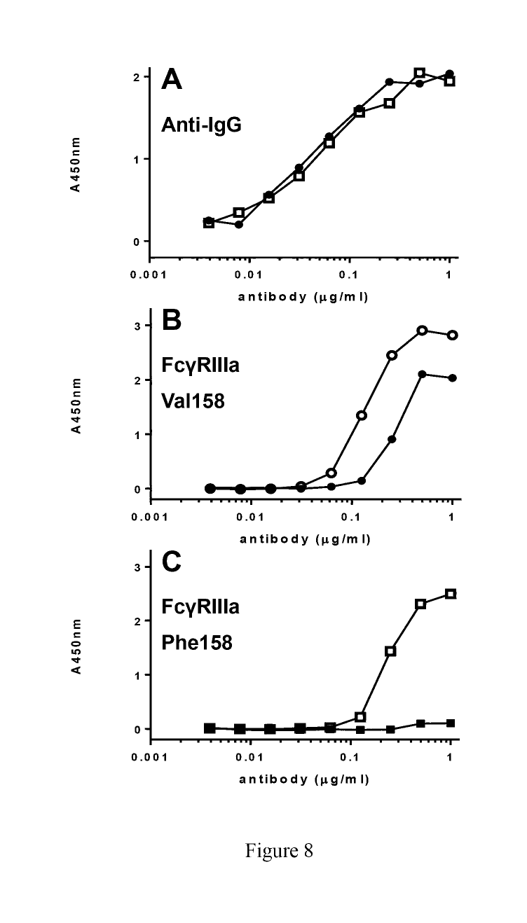

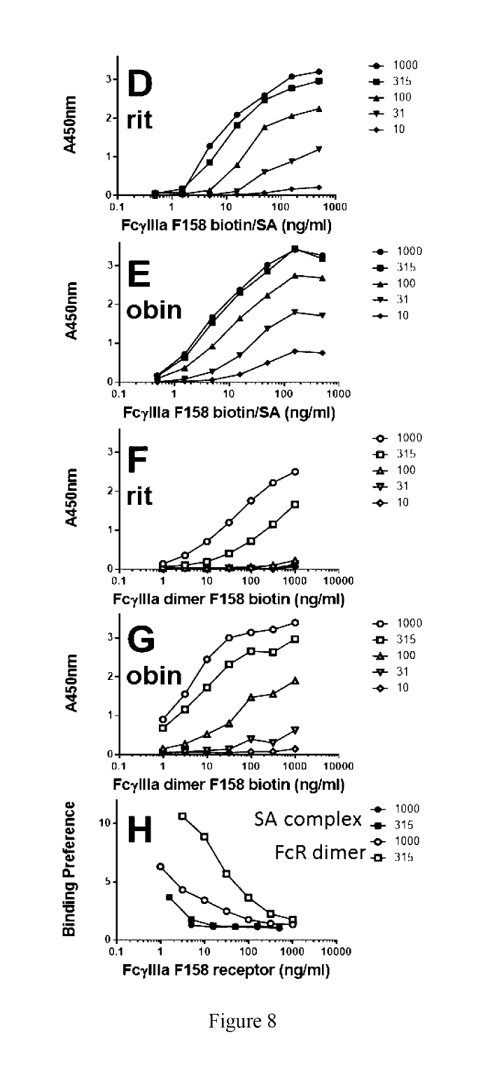

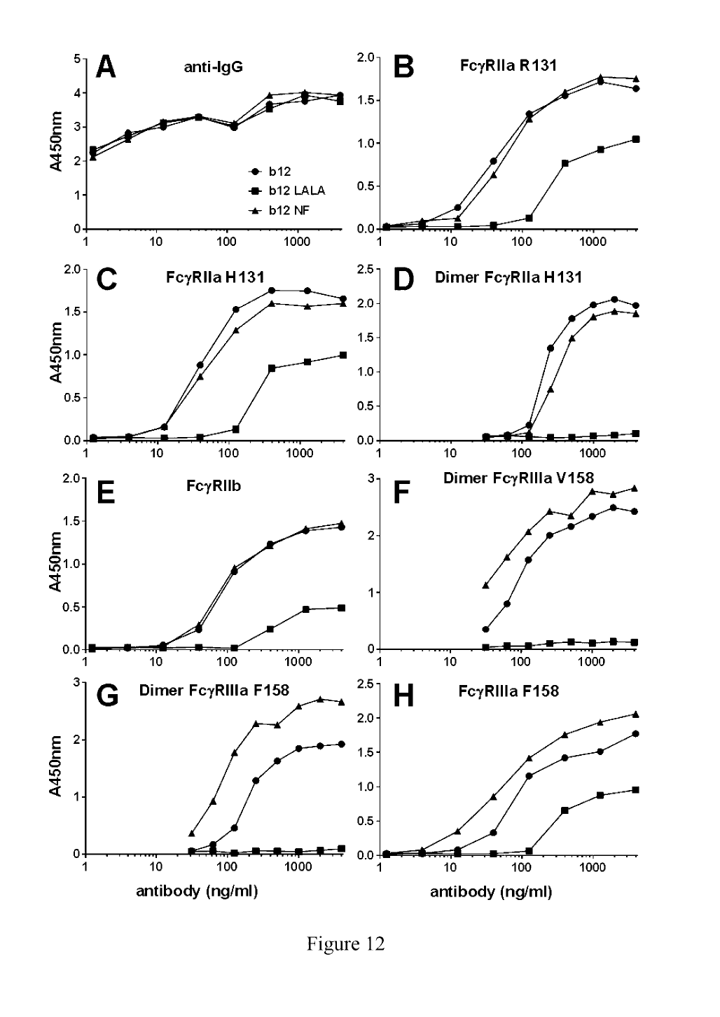

The multimer, binding assay and method of the present disclosure may be used to distinguish IgG subclasses (eg IgG1, IgG2, IgG3, IgG4) on the basis of their FcγR reactivity, as for example, FcγRIIa-H131 binds IgG2 while other FcγRs bind IgG2 poorly. In an embodiment, the multimer, binding assay and method can be used to distinguish between antibody mutants that affect FcR binding activity, including changing the typical Fc binding hierarchy such as (eg IgG) antibody mutants. In an embodiment, they can be used to screen a group of antibodies that bind to a specific antigen to identify an antibody that has a desired FcR binding activity and, accordingly, has a desired immune effector function. When variants of IgG1 that influence FcγR binding were evaluated in the FcγR dimer assays, known effects of modification of IgG were well recapitulated in the assay. Mutation of the lower hinge of IgG1, LL(234-235)AA (aka LALA mutant), a modification known to greatly diminish FcγR binding activity, as was recapitulated in the binding assays with dimeric FcγRIIa and FcγRIIIa binding only weakly to IgG1-LALA:ICs (FIG. 12B-H). In contrast, afucosyl-IgG1 has enhanced binding activity for FcγRIIIa as demonstrated with the receptor dimer assay (FIG. 8B-C; 10C-H) and was most apparent with the low affinity Phe158 allelic form of the receptor. Thus, the FcγR assays are useful for discriminating both activity enhancing and diminishing variants of IgG. FcγRIIIa is considered to be the major mediator of ADCC by NK cells and is also expressed on the CD16+ subset of monocytes and macrophages. The expression FcγRIIa on a wide range of leukocyte cells types allows for the involvement in a variety of aspects of antibody mediated immunity. The FcγRIIb expressed on B cells is a major target for the regulation of immunity. FcγRIIb is also expressed at varying levels on many myeloid cell types.

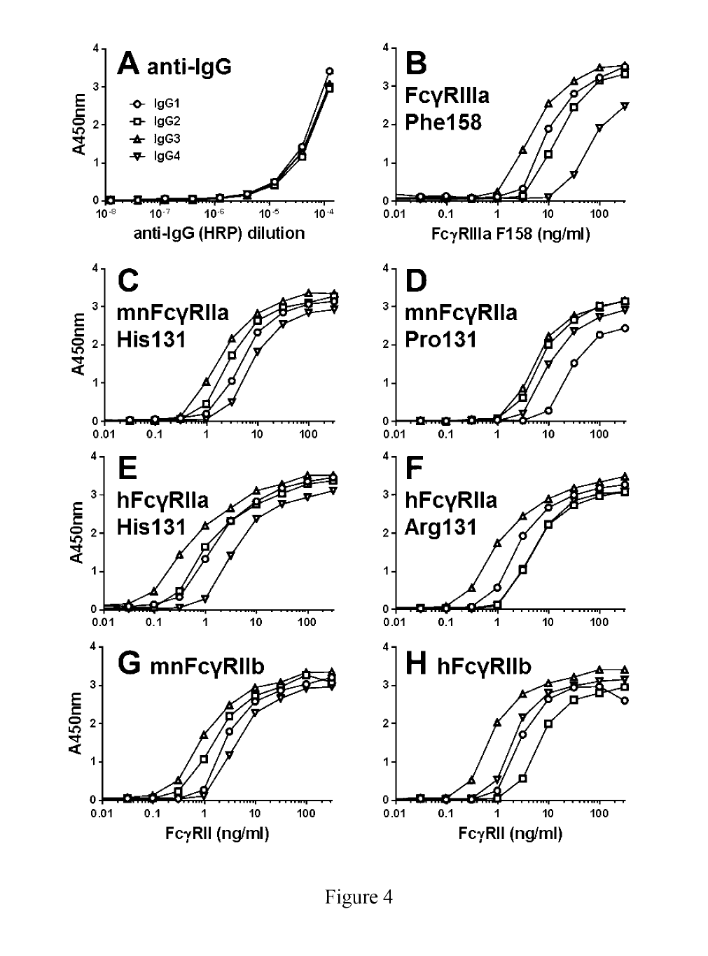

Therapeutic antibodies that block or neutralise target antigens (eg cytokines such as IL-2, receptors such as IL-6R, CD markers, such as CD28) or Fc fusion proteins (such as CTLA-4-Ig fusion protein) are often required to have no FcγR binding activity to avoid off target effects. Sometimes with high target density, such that many antibodies or Fc fusion proteins bind the target so they are have close proximity to one another, highly multivalent FcγR interactions with Fcs are possible. Measuring the decreased intrinsic binding activity of antibody/Fc mutants can give a false impression of complete abrogation of binding. Under high valency of interaction (ie avidity) such antibody/Fc mutants can display significant binding activity. Complexing biotin labelled FcγR ectodomain monomers with streptavidin generates high avidity FcγR complexes for investigating such high valency (ie avidity) interactions. While the IgG4 constant region may be considered backbone with null or low effector function FIG. 4 clearly shows that IgG4 displayed at high density binds well in-fact to all the FcγRs tested when the receptors are arrayed by complexing with streptavidin. Likewise while the LALA mutant of IgG1 may be considered an FcR binding null variant of IgG, FIG. 12B, C, E, H clearly show that when arrayed at high multiplicity by complexing with streptavidin this mutant IgG binds well, although less than wild type IgG1, to the human receptors FcγRIIa H131 (FIG. 12B), FcγRIIa R131 (FIG. 12C), FcγRIIb (FIG. 12E) and FcγRIIIa V158 (FIG. 12H). In contrast the binding of the LALA mutant IgG1 to the FcγR dimers, with well defined valency (ie since they are dimers) is only just detectable. This poor binding of the dimer proteins to LALA mutant IgG1 is demonstrated for FcγRIIa H131 dimer (FIG. 12D), FcγRIIIa V158 (FIG. 12F), FcγRIIIa F158 (FIG. 12G).

In embodiments, the multimer, binding assay and method of the present disclosure may be used to, at least:
Identify IgG antibodies that that selectively bind one Fc receptor type over another;
Identify IgG antibodies that following antigen binding have topology that appropriately or optimally presents the Fc to permit Fc binding with a Fe binding region derived from a particular FcR;
Evaluate FcR binding activity in antibodies derived following vaccination against a specific antigen for predicting protective efficacy of vaccine responses;
Evaluate FcR binding activity in antibodies derived following exposure to a pathogen to determine protective efficacy of pathogen exposure;
Identify antibodies with mutations or modifications associated with altered FcR binding;
Permit tailoring of therapeutic antibodies for administration to patients known to have particular FcγR allelic variants;
Determine epitope proximity mapping; and
Predict cellular activation

In an embodiment, the multimer, binding assay and method of the present disclosure may be used to identify or screen antibodies with antibodies with mutations or modifications associated with altered FcR binding. For example, afucosylated antibodies bind FcγRIII multimers with a higher binding activity compared to antibodies with normal glycosylation patterns.

The binding assay and method of the present disclosure may be suitable for the assessment of human or humanised target antibodies, but the present disclosure also encompasses the assessment of target antibodies from animals of commercial, scientific, medical or personal significance, such as non-human primates and other mammals such as laboratory animals, livestock, exotic animals, and companion animals (including monkeys, race horses, donkeys, sheep, cattle, goats, pigs, lions, tigers, elephants, dogs, cats, rabbits, guinea pigs, rats and mice, etc).

The invention is hereinafter described by way of the following non-limiting examples and accompanying figures.

### EXAMPLE

### Example 1 FcγR Dimers Probe Activation of Immune Cells by IgG

### Materials and Methods

Reagents

The following reagents were used in the experiments described in this example: F96 Nunc maxisorp plate #4424.; Costar Serocluster 96 well U bottom plate #2797; Albumin, bovine Fraction V. Sigma #A-9418; Pierce High Sensitivity Streptavidin HRP conjugate 0.5 ml (1 mg/ml), ThermoScientific #21130; IgG capture reagent: AffiniPure F(ab′)₂ fragment goat anti-human IgG, F(ab′)₂ fragment specific, Jackson ImmunoResearch Laboratories Inc #109-006-097. (lot 120688, 1.3 mg/ml); IVIg: Intragam Pm (intravenous immunoglobulin (normal IgG) (6%) 12 g/200 ml CSL Biotherapies AUST R 68633; IgG1, Kappa from human myeloma Sigma-Aldrich#15154; IgG2, Kappa from human myeloma Sigma-Aldrich#15404; IgG3, Kappa from human myeloma Sigma-Aldrich#15654; IgG4, Kappa from human myeloma Sigma-Aldrich#14639; Polyclonal rabbit anti-human IgG-HRP Dako #P0214; and TMB Single solution 500 ml, Life Technologies #2023 (ELISA substrate).

Summary of Receptor Construction

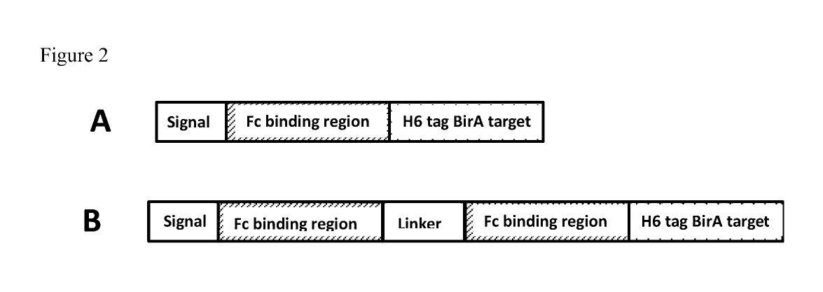

The basic format of the FcR monomer or FcR fusion dimer polypeptides is outlined schematically in FIG. 2A and FIG. 2B.

Briefly, monomer forms were generated as a single polypeptide with an appropriate signal sequence, such as the signal sequence derived from an FcγR such as FcγRIIa or from the human IgG. The signal sequence is fused to an Fc binding region such as an FcγR ectodomain which is fused to a stretch of sequence containing amino acid tag for purification of the recombinant protein, such as a hexa-histidine tag and a sequence for the oligomerisation or for labelling of the expressed polypeptide such as the BirA ligase target sequence for biotinylation. The multivalent fusion polypeptides such as the FcR dimers were generated with an appropriate signal sequence, which is fused to an Fc binding region fused to a linker that connects to a second Fc binding region which in turn is fused to a stretch of sequence containing amino acid for purification of the recombinant protein, such as a hexa-histidine tag and a sequence for the for labelling of the expressed polypeptide such as the BirA ligase target sequence for biotinylation. These polypeptides can be generated by a person skilled in the art by the construction of cDNA using standard laboratory molecular genetics techniques such as PCR or synthesised as codon optimised cDNA or as synthetic polypeptides. The amino acid sequences of the monomer and dimer polypeptides forms of the human and macaque polypeptides are provided below as SEQ ID NOS: 10-22. In the sequences, the polymorphic key residues are shown as a single bold and underlined amino acid residue and for the dimer proteins, the linking sequence between the two tandem FcR binding regions that is derived from an FcR membrane proximal region is shown in bold. The N-terminal leader sequences are underlined and the C-terminal hexahistidine tag and BirA ligase target sequence is underlined.

The amino acid sequence of Human rsFcγRIIa H131 monomer is provided at SEQ ID NO: 10.

| (SEQ ID NO: 10) | |
|---|---|
| 1 | METQMSQNVC PRNLWLLQPL TVLLLLASAD SQAAAPPKAV LKLEPPWINV |
| 51 | LQEDSVTLTC QGARSPESDS IQWFHNGNLI PTHTQPSYRF KANNNDSGEY |
| 101 | TCQTGQTSLS DPVHLTVLSE WLVLQTPHLE FQEGETIMLR CHSWKDKPLV |
| 151 | KVTFFQNGKS QKFSHLDPTF SIPQANHSHS GDYHCTGNIG YTLFSSKPVT |
| 201 | ITVQVPSMGP GSSSHHHHHH PGGGLNDIFE AQKIEWHE |

The amino acid sequence of Human rsFcγRIIa H131 dimer is provided at SEQ ID NO: 11.

| (SEQ ID NO: 11) | |
|---|---|
| 1 | METQMSQNVC PRNLWLLQPL TVLLLLASAD SQAAAPPKAV LKLEPPWINV |
| 51 | LQEDSVTLTC QGARSPESDS IQWFHNGNLI PTHTQPSYRF KANNNDSGEY |
| 101 | TCQTGQTSLS DPVHLTVLSE WLVLQTPHLE FQEGETIMLR CHSWKDKPLV |
| 151 | KVTFFQNGKS QKFSHLDPTF SIPQANHSHS GDYHCTGNIG YTLFSSKPVT |
| 201 | ITVQVPSMGS SSPVAPPKAV LKLEPPWINV LQEDSVTLTC QGARSPESDS |
| 251 | IQWFHNGNLI PTHTQPSYRF KANNNDSGEY TCQTGQTSLS DPVHLTVLSE |
| 301 | WLVLQTPHLE FQEGETIMLR CHSWKDKPLV KVTFFQNGKS QKFSHLDPTF |
| 351 | SIPQANHSHS GDYHCTGNIG YTLFSSKPVT ITVQVPSMGP GSSSHHHHHH |
| 401 | PGGGLNDIFE AQKIEWHE |

The amino acid sequence of Human rsFcγRIIa Rβ1 monomer is provided at SEQ ID NO: 12.

| (SEQ ID NO: 12) | |
|---|---|
| 1 | MVLSLLYLLT ALPGILSAAP PKAVLKLEPP WINVLQEDSV TLTCQGARSP |
| 51 | ESDSIQWFHN GNLIPTHTQP SYRFKANNND SGEYTCQTGQ TSLSDPVHLT |
| 101 | VLSEWLVLQT PHLEFQEGET IMLRCHSWKD KPLVKVTFFQ NGKSQKFSRL |
| 151 | DPTFSIPQAN HSHSGDYHCT GNIGYTLFSS KPVTITVQVP SMGSSSPGSS |
| 201 | SHHHHHHPGG GLNDIFEAQK IEWHE |

The amino acid sequence of Human rsFcγRIIa Rβ1 dimer is provided at SEQ ID NO: 13.

| (SEQ ID NO: 13) | |
|---|---|
| 1 | MVLSLLYLLT ALPGILSAAP PKAVLKLEPP WINVLQEDSV TLTCQGARSP |
| 51 | ESDSIQWFHN GNLIPTHTQP SYRFKANNND SGEYTCQTGQ TSLSDPVHLT |
| 101 | VLSEWLVLQT PHLEFQEGET IMLRCHSWKD KPLVKVTFFQ NGKSQKFSRL |
| 151 | DPTFSIPQAN HSHSGDYHCT GNIGYTLFSS KPVTITVQVP SMGSSSPAAP |
| 201 | PKAVLKLEPP WINVLQEDSV TLTCQGARSP ESDSIQWFHN GNLIPTHTQP |
| 251 | SYRFKANNND SGEYTCQTGQ TSLSDPVHLT VLSEWLVLQT PHLEFQEGET |
| 301 | IMLRCHSWKD KPLVKVTFFQ NGKSQKFSRL DPTFSIPQAN HSHSGDYHCT |
| 351 | GNIGYTLFSS KPVTITVQVP SMGSSSPGSS SHHHHHHPGG GLNDIFEAQK |
| 401 | IEWHE |

The amino acid sequence of Human rsFcγRIIb monomer is provided at SEQ ID NO: 14.

| (SEQ ID NO: 14) | |
|---|---|
| 1 | MVLSLLYLLT ALPGILSAAP PKAVLKLEPQ WINVLQEDSV TLTCRGTHSP |
| 51 | ESDSIQWFHN GNLIPTHTQP SYRFKANNND SGEYTCQTGQ TSLSDPVHLT |
| 101 | VLSEWLVLQT PHLEFQEGET IVLRCHSWKD KPLVKVTFFQ NGKSKKFSRS |
| 151 | DPNFSIPQAN HSHSGDYHCT GNIGYTLYSS KPVTITVQAP SSSPMGPGSS |
| 201 | SHHHHHHPGG GLNDIFEAQK IEWHE |

The amino acid sequence of Human rsFcγRIIb dimer is provided at SEQ ID NO: 15.

| (SEQ ID NO: 15) | |
|---|---|
| 1 | MVLSLLYLLT ALPGILSAAP PKAVLKLEPQ WINVLQEDSV TLTCRGTHSP |
| 51 | ESDSIQWFHN GNLIPTHTQP SYRFKANNND SGEYTCQTGQ TSLSDPVHLT |
| 101 | VLSEWLVLQT PHLEFQEGET IVLRCHSWKD KPLVKVTFFQ NGKSKKFSRS |
| 151 | DPNFSIPQAN HSHSGDYHCT GNIGYTLYSS KPVTITVQAP SSSPMGPAAP |
| 201 | PKAVLKLEPQ WINVLQEDSV TLTCRGTHSP ESDSIQWFHN GNLIPTHTQP |
| 251 | SYRFKANNND SGEYTCQTGQ TSLSDPVHLT VLSEWLVLQT PHLEFQEGET |
| 301 | IVLRCHSWKD KPLVKVTFFQ NGKSKKFSRS DPNFSIPQAN HSHSGDYHCT |
| 351 | GNIGYTLYSS KPVTITVQAP SSSPMGPGSS SHHHHHHPGG GLNDIFEAQK |
| 401 | IEWHE |

The amino acid sequence of Human rsFcγRIIIa V158 monomer is provided at SEQ ID NO: 16.

| (SEQ ID NO: 16) | |
|---|---|
| 1 | MVLSLLYLLT ALPGISTEDL PKAVVFLEPQ WYRVLEKDSV TLKCQGAYSP |
| 51 | EDNSTQWFHN ESLISSQASS YFIDAATVDD SGEYRCQTNL STLSDPVQLE |
| 101 | VHIGWLLLQA PRWVFKEEDP IHLRCHSWKN TALHKVTYLQ NGKGRKYFHH |
| 151 | NSDFYIPKAT LKDSGSYFCR GLVGSKNVSS ETVNITITQG PSMGSSSPGP |
| 201 | GSSSHHHHHH PGGGLNDIFE AQKIEWHE |

The amino acid sequence of Human rsFcγRIIIa V158 dimer is provided at SEQ ID NO: 17. The PSMGSSSP is from the amino acid linker from the rsFcγRIIa binding region dimer:

| (SEQ ID NO: 17) | |
|---|---|
| 1 | MVLSLLYLLT ALPGISTEDL PKAVVFLEPQ WYRVLEKDSV TLKCQGAYSP |
| 51 | EDNSTQWFHN ESLISSQASS YFIDAATVDD SGEYRCQTNL STLSDPVQLE |
| 101 | VHIGWLLLQA PRWVFKEEDP IHLRCHSWKN TALHKVTYLQ NGKGRKYFHH |
| 151 | NSDFYIPKAT LKDSGSYFCR GLVGSKNVSS ETVNITITQG PSMGSSSPSE |
| 201 | DLPKAVVFLE PQWYRVLEKD SVTLKCQGAY SPEDNSTQWF HNESLISSQA |
| 251 | SSYFIDAATV DDSGEYRCQT NLSTLSDPVQ LEVHIGWLLL QAPRWVFKEE |
| 301 | DPIHLRCHSW KNTALHKVTY LQNGKGRKYF HHNSDFYIPK ATLKDSGSYF |
| 351 | CRGLVGSKNV SSETVNITIT QGPSMGSSSP GPGSSSHHHH HHPGGGLNDI |
| 401 | FEAQKIEWHE |

The amino acid sequence of Human rsFcγRIIIa F158 monomer is provided at SEQ ID NO: 18.

| (SEQ ID NO: 18) | |
|---|---|
| 1 | MVLSLLYLLT ALPGISTEDL PKAVVFLEPQ WYRVLEKDSV TLKCQGAYSP |
| 51 | EDNSTQWFHN ESLISSQASS YFIDAATVDD SGEYRCQTNL STLSDPVQLE |
| 101 | VHIGWLLLQA PRWVFKEEDP IHLRCHSWKN TALHKVTYLQ NGKGRKYFHH |
| 151 | NSDFYIPKAT LKDSGSYFCR GLFGSKNVSS ETVNITITQG PSMGSSSPGP |
| 201 | GSSSHHHHHH PGGGLNDIFE AQKIEWHE |

The amino acid sequence of Human rsFcγRIIIa F158 dimer is provided at SEQ ID NO: 19:

| (SEQ ID NO: 19) | |
|---|---|
| 1 | MVLSLLYLLT ALPGISTEDL PKAVVFLEPQ WYRVLEKDSV TLKCQGAYSP |
| 51 | EDNSTQWFHN ESLISSQASS YFIDAATVDD SGEYRCQTNL STLSDPVQLE |
| 101 | VHIGWLLLQA PRWVFKEEDP IHLRCHSWKN TALHKVTYLQ NGKGRKYFHH |
| 151 | NSDFYIPKAT LKDSGSYFCR GLFGSKNVSS ETVNITITQG PSMGSSSPSE |
| 201 | DLPKAVVFLE PQWYRVLEKD SVTLKCQGAY SPEDNSTQWF HNESLISSQA |
| 251 | SSYFIDAATV DDSGEYRCQT NLSTLSDPVQ LEVHIGWLLL QAPRWVFKEE |
| 301 | DPIHLRCHSW KNTALHKVTY LQNGKGRKYF HHNSDFYIPK ATLKDSGSYF |
| 351 | CRGLFGSKNV SSETVNITIT QGPSMGSSSP GPGSSSHHHH HHPGGGLNDI |
| 401 | FEAQKIEWHE |

The amino acid sequence of macaque rsFcγRIIa-His131, including hexahistidine and BirA target sequence tags, is provided at SEQ ID NO: 20.

| (SEQ ID NO: 20) | |
|---|---|
| 1 | METQMSQNVC PGNLWLLQPL TVLLLLASAD SQTAPPKAVL KLEPPWINVL |
| 51 | REDSVTLTCG GAHSPDSDST QWFHNGNLIP THTQPSYRFK ANNNDSGEYR |
| 101 | CQTGRTSLSD PIHLTVLSEW LALQTPHLEF REGETIMLRC HSWKDKPLIK |
| 151 | VTFFQNGISK KFSHMDPNFS IPQANHSHSG DYHCTGNIGY TPYSSKPVTI |
| 201 | TVQVPSVGSS SPGPGSSSHH HHHHPGGGLN DIFEAQKIEW HE |

The amino acid sequence of macaque rsFcγRIIa-Pro131 hexahistidine and BirA target sequence tagged, is provided at SEQ ID NO: 21.

| (SEQ ID NO: 21) | |
|---|---|
| 1 | METQMSQNVC PGNLWLLQPL TVLLLLASAD SQTAPPKAVL KLEPPWINVL |
| 51 | REDSVTLTCG GAHSPDSDST QWFHNGNLIP THTQPSYRFK ANNNDSGEYR |
| 101 | CQTGRTSLSD PIHLTVLSEW LALQTPHLEF REGETIMLRC HSWKDKPLIK |
| 151 | VTFFQNGISK KFSPMDPNFS IPQANHSHSG DYHCTGNIGY TPYSSKPVTI |
| 201 | TVQVPSVGSS SPGPGSSSHH HHHHPGGGLN DIFEAQKIEW HE |

The amino acid sequence of the macaque rsFcγRIIb hexahistidine and BirA target sequence tagged, is provided at SEQ ID NO: 22.

| (SEQ ID NO: 22) | |
|---|---|
| 1 | MGILSFLPVL ATESDWADCK SPQPWGHMLL WTAVLFLAPV AGTPAPPKAV |
| 51 | LKLEPPWINV LREDSVTLTC GGAHSPDSDS TQWFHNGNLI PTHTQPSYRF |
| 101 | KANNNDSGEY RCQTGRTSLS DPVHLTVLSE WLALQTPHLE FREGETIMLR |
| 151 | CHSWKDKPLI KVTFFQNGIS KKFSHMDPNF SIPQANHSHS GDYHCTGNIG |
| 201 | YTPYSSKPVT ITVQVPSMGS SSPGPGSSSH HHHHHPGGGL NDIFEAQKIE |
| 251 | WHE |

Human Fc Receptor Reagents

The following human Fc receptor reagents were prepared in and used in this example:
Biotinylated purified FcγRIIa His131 ectodomain dimer.
Biotinylated purified FcγRIIa Arg131 ectodomain dimer.
Biotinylated purified FcγRIIb ectodomain dimer.
Biotinylated purified FcγRIIIa Val158 ectodomain dimer.
Biotinylated purified FcγRIIIa Phe158 ectodomain dimer.
Biotinylated purified FcγRIIa His131 ectodomain monomer.
Biotinylated purified FcγRIIa Arg131 ectodomain monomer.
Biotinylated purified FcγRIIb ectodomain monomer.
Biotinylated purified FcγRIIIa Val158 ectodomain monomer.
Biotinylated purified FcγRIIIa Phe158 ectodomain monomer.

Macaca Nemestrina Fc Receptor Reagents

The following Macaca nemestrina Fc receptor reagents were used in this example:
Biotinylated purified mnFcγRIIa His131 ectodomain monomer.
Biotinylated purified mnFcγRIIa Pro131 ectodomain monomer.
Biotinylated purified mnFcγRIIb ectodomain monomer.
   The nucleotide and/or amino acid sequences for mnFcγRIIa and mnFcγRIIb are as described by Trist et al., 2014.

Generation of BirA Ligase-Expressing Cell Lines

A pIREShygro expression vector containing the BirA ligase with a C-terminal ER targeting sequence (insert from pDisplay-BirA-ER a gift from Alice Ting (Addgene plasmid #20856; Addgene, Cambridge, Mass., United States of America) (Howarth et al., 2005) was transfected using lipofectamine 2000 into HEK293EBNA cells and Expi293F™ cells (Gibco LifeTechnologies) and transfectants were selected with 100 μg/ml hygromycin and maintained in 50 μg/ml hyuyomycin. This resulted in the production of HEK293EBNA-BirA cells or Expi293F™-BirA cells used below.

FcγRIIa Expression Constructs

FcγRIIa H131 Monomer and Dimer Expression Constructs.

All PCR reactions were performed using polymerase Pwo (Roche) or accuprime Pfx polymerase (Invitrogen, Life Technologies). All other DNA modifying enzymes were from New England Biolabs. The construction of a vector encoding monomeric rsFcγRIIa with a hexahistidine and biotin ligase target tag used amplification from the cDNA encoding FcγRIIa (clone Hu3.0 which includes an unique BamH1 site, Hibbs et al., 1988) and the product ligated to a codon-optimised sequence encoding a hexahistidine tag and biotin ligase target sequence (GenScript USA) so that the C-terminal sequence was GPGSSSHHHHHHPGGGLNDIFEAQKIEWHE such that the underlined residue corresponds to Gly175 of FcγRIIa (ie Gly211 of the precursor; ref NP_001129691). To make a dimerised receptor construct, the clone Hu3.0 was amplified using the equivalent of primers GTAGCTCCCCAAA GGCTG and GGGTGAAGAGCTGCCCATG (GGG corresponding to the antisense for the codon of FcγRIIa Pro179, ie Pro215 of the precursor) and the blunt product was ligated together with T4 ligase and a correctly orientated tandem dimer of the receptor ectodomain subcloned. Using standard molecular biology, a BamHI fragment from this construct was subcloned into the unique BamHI site in the monomeric vector to produce a vector comprising the endogenous FcγRIIa leader, the dimeric ectodomain sequence described above and the C-terminal GPGSSSHHHHHHPGGGLNDIFEAQKIEWHE tag. The monomeric or dimeric rsFcγRIIa proteins were expressed from pAPEX-3p-X-DEST (pBAR424) or pCR3-DEST as described previously (Patel et al., 2010) except using HEK293EBNA-BirA cells or Expi293F™-BirA cells respectively. Expression using Expi293F™-BirA cells followed the manufacturers' instructions for Expi293F™ cells (Invitrogen). All hexahistidine tagged proteins were purified by affinity chromatography on Talon Superflow (BD Biosciences) as described previously (Ramsland et al., 2007).

FcγRIIIa Monomer and Dimer Expression Constructs.

Codon-optimised sequences encoding the ectodomains of rsFcγRIIIa Val158 and Phe158 with the N-terminal sequence

MVLSLLYLLTALPGISTEDLPKAVVFL

and the C-terminal sequence

QGPSMGSSSPGPGSSSHHHHHHPGGGLNDIFEAQKIEWHE

(underlined Q corresponds to residue Gln172, or Gln191 of the precursor, ref NP_000560) were synthesised by GeneArt (Invitrogen) and transposed by BP clonase reaction into pENTR1A (Invitrogen). The native protein leader sequences in these constructs were replaced with

MVLSLLYLLTALPGIST

and validated using Signal P (Petersen et al., 2011). PCR amplification with the primer pair AGCGAGGACCTGCCTAAGGCCGT and CGGAGAACTAGAGCCCATGCTG and with the primer pair CGGAGAACTAGAGCCCATGCTG and GGGCCTGGCAGCTCCTCTC generated an ectodomain and ectodomain in the vector product respectively which were ligated to generate the hexahistidine and BirA ligase target tagged FcγRIIIa ectodomain dimer sequences. The underlined residues of

QGPSMGSSSPSE

were encoded by the linking sequence between the tandem ectodomains, and Q corresponds to residue Gln172 of the first ectodomain and E corresponds to residue Glu1 of the tandem ectodomain. After sequence validation, LR clonase reactions (Invitrogen) with pCR3-DEST generated expression vectors for monomeric and dimeric forms of FcγRIIIa, Val158 and Phe158. Expression of these pCR3 based constructs used Expi293F™-BirA cells.

Macaque FcγR Monomer Expression Constructs.

Standard molecular biology techniques and the cDNA templates described in Trist et al., 2014 were used to create C-terminal hexahistidine and BirA target sequence tagged FcγR binding regions of mnFcγRIIa His131, mnFcγRIIa Pro131, and mnFcγRIIb.

Biotinylation of Recombinant Soluble FcR

Biotinylation of the FcR polypeptides described above was achieved by transfection of the expression plasmids into cells that express BirA ligase resulting in the secretion of FcR proteins that were biotinylated during their cellular production. Thus, the FcR protein expression vectors were transfected into HEK293EBNA-BirA cells or Expi293F™-BirA cells as described above.

Antibody Expression Constructs

The production of a chimeric IgG1 comprising a mouse leader and VH sequence (from TIB142; American Type Culture Collection) joined to a human IgG1 constant region sequence and a corresponding mouse kappa L chain (TIB142) has been described previously (Patel et al., 2010). Chimeric anti-TNP kappa light chain consisting of TIB142VH and human constant kappa was produced from a codon opsonised construct synthesized by Bioneer Pacific (Kew, Australia). An IgG4 chimeric heavy chain comprising TIB142 leader and VH and IgG4 constant (Bioneer Pacific) sequence was codon optimised and synthesised and transferred into pCR3-DEST by LR clonase reaction. Likewise, a TNP specific chimeric sequence using an IgG2 genomic constant region (Accession number J00230) was made using pCR3 and standard molecular biology techniques.

Binding Assay Using Biotinylated FcγR Genetic Fusion Dimer

An IgG capture reagent, either F(ab′)₂ goat anti-human IgG, F(ab)₂ (10 μg/ml) or TNP-BSA (20 μg/ml), or antigen (typically 1 μg/ml; for example HIV envelope gp140 as described in Center et al., 2009) was prepared in PBS and adsorbed (50 ul per well) to plates (Nunc Maxisorp) and subsequently blocked with PBS containing 1 mM EDTA and 1% (w/v) bovine serum albumin (BSA, fraction V, Sigma). IgG samples (typically diluted from a starting concentration of 5 to 1 μg/ml were incubated with the IgG capture reagent, TNP-BSA or antigen coated wells for 1 hour at 37° C. After washing the plate by five times filling and emptying with PBS containing 0.05% Tween 20, the antibody bound plates were incubated with 0.2 μg/ml biotinylated purified FcγRIIa-dimer, or 0.1 μg/ml biotinylated purified FcγRIIIa-dimer, in PBS diluent containing 1 mM EDTA, 0.05% Tween20 and 1% (w/v) bovine serum albumin for 1 hour at 37° C. After five cycles of filling and emptying with wash buffer, high sensitivity (hs) HRP-streptavidin (Pierce) 1/10,000 in diluent buffer was added for 1 hour at 37° C. followed by eight cycles of filling and emptying with wash buffer and development with TMB Single solution (Life Technologies). The reaction was stopped by the addition of an equal volume of 1M HCl, and absorbance at A450 nm was then immediately determined as delayed determination of absorbance can produce an apparent “prozone effect” artefact as precipitation of the high colorimetric product occurs at high concentration. In an alternative to measuring the FcR binding activity of the IgG immune complexes, IgG was also detected using polyclonal rabbit anti-human IgG-HRP (Agilent Technologies-Dako, Denmark) at 1/10 000 dilution.

Binding Assay Using Streptavidin-HRP Conjugate Complexed Biotinylated FcγR Monomer

Assays were performed with the biotinylated FcγR monomer proteins (1 μg/ml) mixed with HRP-streptavidin (Pierce) 1/10,000 to form receptor/streptavidin-HRP complexes and otherwise used the procedure described for the dimeric receptor assay.

FcγRIIa and FcγRIIIa Dimer Binding to IgG Subclasses

Capture reagent and human immunoglobulin Intragam (IgG) standard were coated onto the ELISA plate as follows. IgG capture reagent, F(ab′)₂ goat anti-human IgG, or F(ab′)₂, was prepared in PBS at 10 μg/ml and incubated at 50 μl per well in Maxisorp ELISA plates in an appropriate layout leaving some wells without capture reagent. 1 μg/ml of the IgG capture reagent has proved to be an optimal concentration for a number of antigens. High antigen density on the plate increases the density of the subsequent opsonising antibodies and the FcR binding activity of even low activity samples. When 10 μg/ml is used, a higher density of human IgG is captured.

Human immunoglobulin for intravenous use (Mg; Intragam P, CSL Limited, Parkville, VIC, Australia; stock 60 mg/ml) was diluted to 5 μg/ml in phosphate buffered saline containing 1 mm EDTA (PBSE) and ½ log dilutions performed starting from this 5 μg/ml standard. The IVIg standard provided a positive control on the plate with a titration of the FcγR dimer activity. The signal from the 5 μg/ml wells can be used as an inter-plate standard to normalise the FcR activities across plates. The plates were incubated overnight at 4° C. or 1 hour at 37° C. or 2 hours at RT. Every well on the plates were blocked by adding 140 μl of PBSE containing 1% (w/v) bovine serum albumin, or PBSE/BSA when testing monoclonal antibodies or mixtures of mAbs binding to antigens; however, when human sera containing antibodies was tested, the plate was blocked with human serum albumin as human sera at low dilution shows significant IgG binding to BSA for some individuals. The plates were then incubated at 37° C., or 1 h or overnight at 4° C. The plates were then washed twice with PBS 0.05% Tween20 to remove the blocking solution. The wells were then filled with wash buffer and aspirated.

IgG1, IgG2, IgG4 myeloma derived IgG were serially diluted 1:1 in PBSE/BSA from 5 μg/ml to 0.039 μg/ml. For IgG3 myeloma samples, the serial dilution ranged from 1.25 μg/ml to 0.010 μg/ml. The dilution series (50 μl) were performed on the IgG capture reagent coated/blocked wells. For antibody bound to antigen, both antigen coated/blocked and control blocked wells were present on the plate.

The plates were then incubated at 37° C. for 1 h (alternatively incubation overnight at 4° C. or 1-2 hours at RT is also appropriate). The plates were then washed with PBS 0.05% Tween to remove the blocking solution. The wells were then filled with wash buffer and aspirated five times. 0.2 μg/ml of the biotinylated purified FcγRIIa-dimer diluted in PBSE/BSA, or 0.1 μg/ml biotinylated purified FcγRIIIa-dimer diluted in PBSE/BSA or 1/10 000 rabbit anti-human IgG-HRP was added to every well of the plate which was then incubated at 37° C. for 1 h. The plate was then washed five or eight times with PBS 0.05% Tween. High sensitivity HRP-streptavidin (Pierce) diluted 1/10,000 in PBSE/BSA was then added to each well, and the plate was incubated at 37° C. for 1 h. The plate was then washed with PBS 0.05% Tween eight times. Remaining buffer was removed from the wells by tapping on a paper towel, and then 50 μl TMB substrate was added. At the appropriate time, judged by observing the intensity of the 5 μg/ml IgG standard, typically 4 to 10 min, colour development was stopped by adding 50 μl 1M HCl. The absorbance at 450 nm was then determined.

FcR binding activity for each sample was calculated by zeroing the absorbance readings on the basis of the “no IgG” control, and then the FcR binding activities were normalised as follows:

Normalised FcR activity=(OD1−OD2)/OD3

### Where:

OD1=OD of FcR dimer binding to antibody;

OD2=OD of FcR background binding to Ab in the absence of the capture reagent (or antigen);

and

OD3=FcR binding to the 5 μg/ml Mg IgG standard.

For sera, FcR activities were normalised to IgG titre, when the FcR reagent was substituted for HRP conjugated anti-IgG (eg Dako at 1/10,000 dilution). The FcR binding activities were correlated to cellular activities such as phagocytosis or ADDC, and the activities of different FcR (eg FcγRIIa to FcgRIIIa) can be correlated to each other.

Curve Fitting and Data Analysis Method

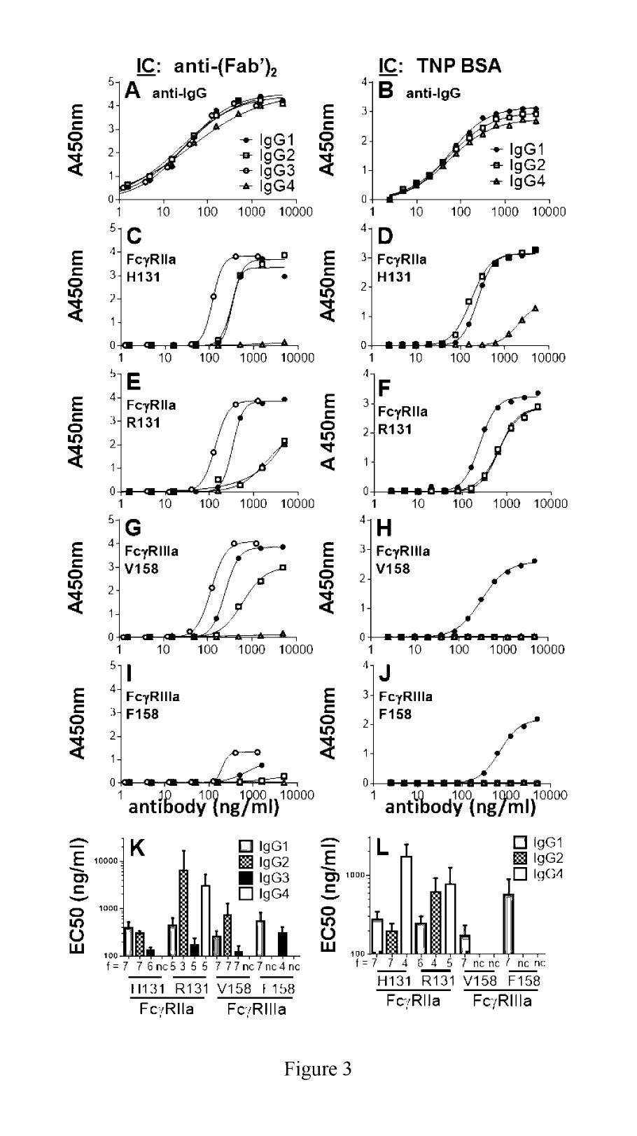

Statistical analysis was performed with Prism GraphPad version 6.05 (GraphPad Software, San Diego, Calif.). Binding data were fitted using Prism software, to log (agonist) vs. response (variable slope, constraining bottom value=0, the top value was allow to vary freely). Cumulative data shown as EC50 with error bars is presented as mean±95% C.I. in FIGS. 3K and L. Curve fitting for some low affinity interactions, with IgG2 and IgG4, were ambiguous and EC50 values were only used from data fitting where r2>0.97. When binding was undetectable, or too weak to enable adequate fitting, the EC50 value was not calculated (annotated in the figures as “nc”).

Binding Assay Using Fucosylated and Afucsoylated Anti-CD20 Antibodies

Binding assays using variant antibodies such as mutants or glycosylation mutants used the same method as for WT IgG molecules. The afucosylated anti-CD20, GAZYVA® (obinutuzumab) (Klein et al., 2013) was obtained from the Alfred Hospital Pharmacy (Prahran, VIC, Australia) and afucosyl and LALA variants of the anti-b12 anti-HIV mAb were as described in Moldt et al., 2012.

Flow Cytometry Binding Assay on Cellular Surfaces

Flow cytometric analysis was performed as previously described (Trist et al., 2014).

### Reagents Used for Flow Cytometry

FACS Buffer (FB) PBS/1% BSA/1.1 mM glucose; Rituximab 20 μg/ml in FB [Pharmacy stock at 12 mg/ml]; Obituzumab 20 μg/ml in FB (Pharmacy stock at 22 mg/ml); PE conjugated anti-CD19 APC conjugated mAB ( 1/10; BD Biosciences); Biotinylated FcR receptor dimer (stock concentrations as follows: FcγRIIa-His131, 1.6 mg/ml; FcγRIIIa-V158, 0.7 mg/ml; FcγRIIIa-F158, 0.5 mg/ml); Streptavidin PE conjugated (0.5 mg/ml stock)(554061, Chemicon); Dilution of goat anti-hIgG Fc FITC (2 mg/ml stock; APF, Chemicon)

Method—

Daudi cells or peripheral blood mononuclear cells (PBMC) were isolated from whole blood as described (Trist et al., 2014) and used at a concentration of 5×10⁶ cells/ml in FACS buffer. CD20 mAbs were diluted to the relevant concentration from 20 μg/ml stock in cold FB and 25 μL added to 25 μl of cells at 5× cells/ml. Cells were incubated on ice for 30 minutes washed twice with cold FB and resuspended in 25 μl of FB. Twenty five microliters of biotinylated FcR dimer was added at the indicated concentration. The cells were incubated on ice for 30 mins, washed twice with cold FB, and re-suspended in Streptavidin PE (25 μl of 1/800 dilution in FB). Cells were incubated for a further 30 mins on ice, washed twice with FB then resuspended in 200 μl of FB and analysed on a BD biosciences Canto flow cytometer. Alternatively, following incubation with anti-CD20 mAbs and washing, the degree of anti-CD20 opsonisation of cells was separately determined by substituting 25 μl of 1/200 dilution of goat anti-hIgG Fc FITC conjugate for FcR dimer in the above protocol. The cells were incubated on ice for 30 minutes, washed twice in cold FB and resuspended in 200 μl of cold FB and analysed on a BD biosciences Canto flow cytometer. For experiments analysing rituximab and FcR interaction on CD19 B cells in PBMC, the anti-CD19 mAB (25 μl at a 1/10 dilution in FB) was added to cells at the same time as the streptavidin-PE conjugate (25 μl of 1/800 dilution in FB). The cells were incubated for 30 mins on ice, washed twice with FB, then resuspended in 200 μl of FB and analysed on a BD biosciences Canto flow cytometer.

Results—

Recombinant soluble (rs) FcγRs tagged with hexahistidine and BirA target sequences were expressed in Expi293-BirA cells and purified using Talon affinity chromatography. The proteins were analysed by 12% polyacrylamide SDS-PAGE (data not shown

It was hypothesised that the dimerisation of the ectodomains of FcγRII or FcγRIII with a flexible linking sequence would permit their avid binding to antibody pairs in immune complexes. The biotin-labelled ectodomain dimers of FcγRIIa His131, FcγRIIa Arg131, FcγRIIIa Val158 and FcγRIIIa Phe158 (and FcγRIIb) were produced in BirA ligase expressing cells. The activities of these human FcγR dimers were characterised by testing their binding to the different human IgG subclasses.

Human rsFcγRII and rsFcγRIIIa Dimers Selectively Bind IgG Subclasses and Distinguish Between Different Types of IC (Anti-Fab Versus Anti-TNP IC)

The data displayed in FIG. 3 is a curve fitting analysis of the binding of dimeric FcγR to human IgG subclasses formed as immune complexes by either the capture of IgG with Fab′2 fragments of an anti-human IgG Fab′2 antibody or by capture with antigen (TNP-BSA).

Human IgG subclasses at concentrations from approximately 500 to 1 ng/ml were formed into immune complexes either by capture with plate bound F(ab′), anti-human F(ab), and myeloma IgG1, IgG2, IgG3, IgG4 proteins (FIG. 3 A, C, E, G, I), or by binding TNP-BSA with recombinant human IgG1, IgG2 and IgG4 subclass antibodies specific for the hapten, trinitrophenol (TNP) (FIG. 3 B, D, F, H, J). These immune complexes were then reacted with HRP-conjugated anti-human IgG (FIG. 3 A, B) or with biotinylated FcγRIIa His131 dimer (FIG. 3 C, D), FcγRIIa Arg131 (FIG. 3 E, F) biotinylated FcγRIIIa Val158 dimer (FIG. 3 G,H), or biotinylated FcγRIIIa Phe158 dimer (FIG. 3 I, J) with subsequent detection by streptavidin-HRP.

The effect of IgG subclass on dimeric rsFcγR binding was investigated using two methods of forming model ICs. Firstly, the capture of IgG by the F(ab′)2 anti-human F(ab′)2 to form ICs (FIG. 3A) results in the presentation of Fc regions of the IgGs in varied orientations for dimeric rsFcγR binding (FIG. 3 C, E, G, I). Secondly, TNP hapten specific recombinant IgG and TNP-BSA forms ICs (FIG. 3B) in which all IgGs are orientated by the same variable domain:hapten interaction and so will display a more uniform presentation of Fcs for dimeric rsFcγR binding (FIG. 3D, F, H, J).

Dimeric rsFcγRIIa His131 binding to human IgG1, IgG2, IgG3 or IgG4 captured using F(ab′)2 anti-human F(ab′)2 to form ICs was most potent to IgG3 complexes, equivalent between IgG1 and IgG2 and least potent to IgG4, for which a binding curve could not be fitted unambiguously (i.e. IgG3>IgG1 IgG2>>IgG4, FIG. 3C). Across experiments, the EC50 values for dimeric rsFcγRIIa His131 binding to Ab pairs within ICs formed with IgG1, IgG2 and IgG3 were defined by 95% confidence intervals of 270-520 ng/ml, 260-340, and 110-160 ng/ml (means of 398, 302 and 134 ng/ml respectively, FIG. 3K). Likewise the dimeric rsFcγRIIa Arg131 bound strongly to IgG3 and IgG1 ICs (FIG. 3E, mean EC50 of 440 and 170 ng/ml respectively) but weakly to both IgG2, in contrast to the FcγRIIa His131, and IgG4 (FIG. 3D, mean EC50 of ˜6 and 3 μg/ml respectively). This hierarchy of binding defined in the receptor dimer assay (FIG. 3K,) is comparable to the reported reactivity of the allelic forms of FcγRIIa as a cell surface receptor.

The nature of the IC also influenced dimeric rsFcγR binding. The binding differed most markedly for the weakest FcR interactions observed. For example, dimeric rsFcγRIIa His131 binding activity was just detectable with ICs formed with anti-human F(ab′)2 with 5 μg/ml IgG4 (FIG. 3C), while binding to TNP-BSA: IgG4 IC the signal was ˜6-fold higher at 5 μg/ml IgG4 (FIG. 3D, FIG. 3L, EC50 of ˜2 μg/ml). For this lowest affinity FcR interaction, antibody presentation in these different forms of ICs profoundly affects receptor binding. For the higher affinity interactions, differences between the two methods of IC formation for FcγR binding were less apparent. For example, the dimeric rsFcγRIIa His131 bound similarly to both the anti-human (Fab′)2:IgG1 and IgG2 ICs (EC50 of 280 and 190 ng/ml respectively, FIG. 3 C, K) and to the TNP-BSA:IgG1 and IgG2 ICs (EC50 of 400 and 300 ng/ml respectively, FIG. 3 D, L).

The dimeric rsFcγRIIa Arg131 bound anti-human F(ab′)2 ICs with the ranking, IgG3>IgG1>>IgG2˜IgG4 (FIG. 3E) and a similar hierarchy, IgG1>IgG2˜IgG4, was apparent with anti-TNP ICs (FIG. 3F). Thus the allelic forms of dimeric rsFcγRIIa recapitulate the IgG subclass binding behaviour of their cellular counterparts and their weak binding to IgG4 is influenced by Fc presentation in different forms of IC.

Analysis of the allelic variants of dimeric rsFcγRIIIa proteins showed, as expected, that the dimeric rsFcγRIIIa Val158 had greater binding activity to IgG1 IC than the lower affinity dimeric rsFcγRIIIa Phe158 (EC50=260 versus 540 ng/ml FIG. 3K; EC50=170 versus 570 ng/ml FIG. 3L). The hierarchy of binding to anti-F(ab′)2: ICs of IgG3>IgG1>IgG2>>IgG4 and TNP BSA-ICs of IgG1>>>IgG2, IgG4=nil (FIG. 3G, I, H, J, K,L, Table II) is largely comparable with binding of ICs of the different IgG subclasses to cell surface expressed FcγRIIIa.

It is notable that differences occur in FcγR binding to ICs made with anti-Fab′2 or TNP-BSA antigen. Using the assay to evaluate FcγRIIIa interactions with anti-TNP IC revealed, similarly to FcγRIIa, that the weakest interactions between dimeric rsFcγRIIIa and ligand were influenced the most by the nature of formation of the IC. The interaction of ICs of IgG2 and IgG4 with the higher (FIG. 3 G, H) and lower affinity (FIG. 3 I, J) alleles of the dimeric rsFcγRIIIa were undetectable with the anti-TNP formed ICs (FIG. 3 H, J) but binding was measurable for ICs of F(ab′)2 anti-human F(ab′)2 with IgG2 binding the dimeric rsFcγRIIIa Val158 (FIG. 3G, EC50=620, 95% C.I.=520-740 ng/ml). Clearly the binding, or non-binding, to IgG subclasses by FcγRIIa (compare IgG4 IC binding in FIG. 3C with 3D) and FcγRIIIa (compare IgG2 IC binding in FIG. 3H with 3G) can be dependent on the nature of the IC.

In summary the dimeric rsFcγRs demonstrate binding equivalent to cell surface FcγRs in, i) the hierarchy of binding to IgG subclasses (e.g. binding to IgG3>other subclasses), ii) the subclass specificity of the polymorphic forms of FcγR (e.g. binding of IgG2 by FcγRIIa His131>FcγRIIa Arg131, and iii) the expected differences in binding strength of the polymorphic forms of FcγR (e.g. FcγRIIIa Val158 versus FcγRIIIa Phe158. In addition, how the Fc is presented influences dimeric rsFcγR binding, especially for low affinity interactions, in particular IgG2 and IgG4 e.g. FcγRIIa His131 with IgG4 ICs (FIG. 3C, D) and FcγRIIIa with IgG2 (FIG. 3G-J).

TABLE 2
Reactivity of FcγRIIa His131 with immune complexes of IgG subclasses

| Receptor Form | IC Form | Binding hierarchy |
|---|---|---|
| Dimers | anti-Fab′2 IC | IgG3 > IgG1~IgG2 >> IgG4 |
| Monomer/SA | anti-Fab′2 IC | IgG3 > IgG1~IgG2 >> IgG4 |
| Dimers | TNP IC | IgG1 ≥ IgG1 >> IgG4 |
| Monomer/SA | TNP IC | IgG1~IgG2 > IgG4 |
| Lux2013* | TNP IC | IgG3~IgG1 > IgG2 ≥ IgG4 |
| Bruhns 2009 | 0.5 ug/ml Fab′2 IC | IgG3 > IgG1 > IgG2 > IgG4 |

Reactivity of FcγRIIIa with immune complexes of IgG subclasses.

| receptor form | IC | Binding hierarchy |
|---|---|---|
| Dimers | anti-Fab′2 IC | IgG3 > IgG1 > IgG2 > IgG4 |
| Monomer/SA | anti-Fab′2 IC | lgG3 > IgG1 > IgG2 > IgG4 |
| Dimers | TNP IC | IgG1 >> IgG4 > IgG2 |
| Monomer/SA | TNP IC | IgG1 > IgG4 > IgG2 |
| Lux2103* | TNP IC | IgG3 ≥ IgG1 ≥ IgG4 ≥ IgG2 |
| Bruhns 2009 | 0.5 ug/ml Fab′2 IC | IgG3 > IgG1 > IgG4 ≥ IgG2 |

Accordingly, the FcγR genetically fused dimer assay conforms to the expected specificity for i) different FcγR types (FcγRII versus FcγRIII), ii) for different polymorphic FcγR (eg FcγRIIIa Val158 versus FcγRIIIa Phe158, and iii) retains hierarchy of binding to IgG subclasses found for cell surface FcγRs (for example, IgG3>other subclasses). It was found that the assay can distinguish between different forms of IgG immune complexes. For example, IgG4 immune complexes formed with F(ab′)₂, anti-human F(ab′)₂ (FIG. 3C) have little binding activity for FcγRIIa His131, whereas TNP-specific IgG4 bound to TNP antigen has, in comparison, substantial FcγRII binding activity.

FcγRII and FcγRIIIa Biotinylated Monomers Complexed with Streptavidin have Less Selective Binding of IgG Subclasses than FcγR Dimers

Next, the biotin-labelled monomer ectodomains of human FcγRIIa His131, FcγRIIa Arg131, FcγRIIb and FcγRIIIa Phe158 and Macaca nemestrina (mn) FcγRIIa His131, mnFcγRIIa Pro131 and mnFcγRIIb were produced in cells expressing biotin ligase. As the genetically fused dimeric ectodomains bound immune complexes, it was hypothesised that complexing these FcγRII or FcγRIII receptor monomer-biotin proteins with streptavidin-HRP would confer on them avid binding to IgG immune complexes. Human IgG subclass myeloma proteins IgG1, IgG2, IgG4 (each at 5 μg/ml) and IgG3 (1.25 μg/ml) were captured using F(ab′)₂ anti-human F(ab′)₂ to form immune complexes and reacted with the indicated dilutions of (FIG. 4 A) HRP-conjugated anti-human IgG or the following FcγR monomer biotin conjugates: (FIG. 4B) hFcγRIIIa Phe158, (FIG. 4C) mnFcγRIIa His131, (FIG. 4D) mnFcγRIIa Pro131, (FIG. 4E) hFcγRIIa His131, (FIG. 4F) hFcγRIIa Arg131, (FIG. 4G) mnFcγRIIb His131, or with (FIG. 4H) hFcγRIIb Arg131.

The binding activities of human and macaque (Macaca nemestrina; mn) rsFcγR-biotin/streptavidin-HRP complexes were characterised by binding to the different human IgG subclasses. A fixed concentration of human IgG subclass myeloma proteins was captured using F(ab′)₂ anti-human F(ab′)2 to form immune complexes and was found to show equivalent reactivity with anti-IgG (FIG. 4A). By titrating the rsFcγR-biotin (for example, FIG. 4B) with a fixed concentration of streptavidin-HRP, different receptor:streptavidin complexes with varied avidities were examined for binding to IgG immune complexes. As these receptors bind with low affinity as monomers, binding increased as the input concentration of the rsFcγR-biotin was increased, with the signal approaching a maximum at 100 ng/ml rsFcγR-biotin. Interestingly, the binding curves of the different IgG subclasses showed most difference at low rsFcγR-biotin input concentration, where the binding avidity would be lowest. At high rsFcγR-biotin input, where the streptavidin would be saturated and binding avidity greatest, the curves effectively converged (especially for rsFcγRII FIG. 4C, E, F, G, H). Accordingly, at low avidity, the affinity differences of FcγRII and FcγRIII for different subclasses has a dominant effect on binding outcome; while at sufficient higher avidity these binding differences converge so that “weaker” and “stronger” receptor subclass interactions become essentially equivalent. For example, huFcγRIIa at 1 ng/ml complexed with streptavidin-HRP, binds IgG1 approximately 5-fold more strongly than IgG4, while at 100 ng/ml, this binding preference is only approximately 1.1-fold (FIG. 4E). Similarly, while binding to IgG4 is barely detectable at a rsFcγRIIa-biotin input less than 1 ng/ml, active receptor complexes were formed at this concentration as binding to IgG3 was readily detected at 1 ng/ml (FIG. 4E, F). Notably, the huFcγRIIa Arg131/streptavidin-HRP complexes showed lower binding activity (FIG. 4F) than the His131 allelomorph (FIG. 4E) at low receptor concentrations.

Comparison of the binding activities of rsFcγR in the streptavidin complexed format demonstrated that low activity combinations of ligand (ie IgG subclass such as IgG4) and receptor (eg FcγRIIa) can result in near equivalent binding outcomes to high activity combinations (eg IgG1 or IgG3 with FcγRIIa or FcγRIIIa) when rsFcγR input is high (ie when the streptavidin/rsFcγR complexes display high stoichiometry of rsFcγR). Further, while streptavidin has up to 4 binding sites for biotin, the molecular state of the HRP conjugate with streptavidin is not specifically defined; however, at sufficient stoichiometry, avid interactions of Fc receptors will result in near equivalent binding of all of the IgG subclasses. This is important as some antigens on cellular targets will be able to form such highly multivalent interactions, which would, for example, lead to significant FcR mediated binding by a therapeutic antibody formatted as an IgG4.

At the low FcγR concentrations, the hierarchies of IgG subclass binding for each receptor is similar to that previously reported for cell surface binding. For example, the His131 form of huFcγRIIa (FIG. 4E) is a superior binder of IgG2 compared to the Arg131 receptor (FIG. 4F). Likewise, for the macaque receptors, the His131 form of mnFcγRIIa has somewhat reduced IgG1 binding (FIG. 4C) compared to huFcγRIIa His131 (FIG. 4E) and the mnFcγRIIa Pro131 (FIG. 4D) has further reduced IgG1 binding. Human huFcγRIIb (FIG. 4H) showed better or equivalent binding to IgG4 and IgG1, whereas for the macaque (mn)FcγRIIb (FIG. 4G), IgG4 was the lowest activity antibody subclass. This indicates that macaque models may underestimate the “in-human” activity of a therapeutic IgG4 formatted antibody. Accordingly, the FcγR-biotin monomers at low concentration complexed with streptavidin (FIG. 4) show species differences in interactions and hierarchies of binding activities for the different IgG subclasses that are similar to the dimer formatted receptors (shown in FIG. 3). At higher receptor concentration, these binding differences were reduced by the higher binding avidity of the streptavidin complexed receptors, which is a useful difference between the two receptor formats.

Accordingly, the oligomers of various human and non-human primate (NHP) monomeric FcγR are highly functional in ligand binding. When receptor concentration is sufficiently high, the discrimination between subclasses is no longer apparent, that is, the discrimination between IgG subclasses is most apparent at low FcγR concentration. Comparative use of FcγR dimers and FcγR oligomers can discriminate between IgG subclass capacity to bind FcγR at low and high levels of receptor.

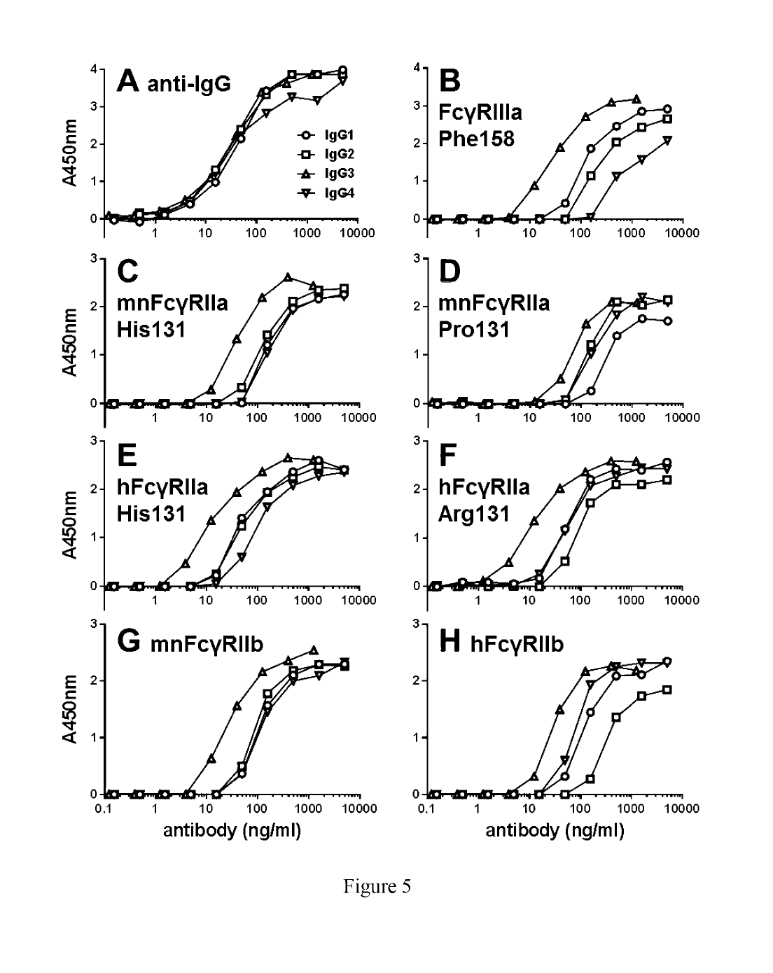

FcγRII and FcγRIIIa biotin monomers in excess complexed with HRP-conjugated streptavidin display selective binding of IgG subclasses is most apparent a low antibody concentration Human IgG subclass myeloma proteins IgG1, IgG2, IgG4 (initially 5 μg/ml) and IgG3 (initially 1.25 μg/ml) were serially diluted as indicated and the concentration series captured using F(ab′)₂ anti-human F(ab′)₂ and reacted with either HRP-conjugated anti-human IgG (FIG. 5A) or the following biotinylated FcγR monomer (at 1 μg/ml) complexed with 1/10,000 diluted HRP-conjugated streptavidin: hFcγRIIIa Phe158 (FIG. 5B); mnFcγRIIa His131 (FIG. 5C); mnFcγRIIa Pro131 (FIG. 5D); hFcγRIIa His131 (FIG. 5E); hFcγRIIa Arg131 (FIG. 5F); mnFcγRIIb His131 (FIG. 5G); or hFcγRIIb Arg131 (FIG. 5H). FcγR oligomers are produced by streptavidin binding with several bioinylated FcγR monomers.

The strength of Fc receptor immune complex interactions can be altered by changes in levels of cellular Fc receptors or in the size of immune complexes. Accordingly, the avidity of the interaction of our purified receptor system was next modified by forming highly avid receptor complexes (oligomers) with an excess rsFcγR-biotin (2 μg/ml) over streptavidin-HRP and the titration of the IgG subclass ligands to limiting concentration (FIG. 5). This approach produced a similar pattern of binding hierarchy for FcγRIIIa (FIG. 5B) as occurred with varying receptor concentration (FIG. 4B). For IgG3, IgG1 and IgG2 binding activities converge towards a maximum above 100 ng/ml IgG, while IgG4 binding only occurs with immune complexes formed above 100 ng/ml and only reaches half maximal binding at 1000 ng/ml. Likewise, the IgG subclass binding hierarchies by the binding various forms of FcγRIIa tested were similar whether the avidity of the interaction was modified by varying the receptor or antibody concentration.

Accordingly, various human and NHP low affinity monomeric FcγR retain the capacity to bind IgG subclasses following oligomerisation (with streptavidin-HRP) as do FcγR dimers; however, the streptavidin oligomerised monomers have greater activity for binding the low affinity IgG subclasses (FIG. 4) than do the FcR dimers (FIG. 3). As such, comparative use of FcγR dimers and FcγR oligomers can discriminate between subclass capacity to bind FcγR at low and high levels of receptor.

Human Biotinylated FcγRII and FcγRIIIa Monomers Complexed with Streptavidin Selectively Bind to Different Antigen: IgG1/IgG2/IgG4 Subclass Immune Complexes

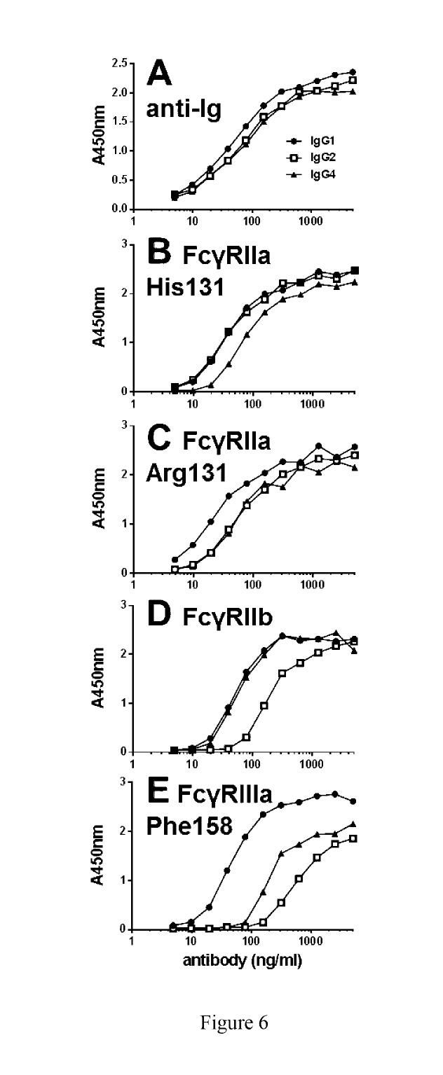

Recombinant human IgG1, IgG2 and IgG4 subclass antibodies specific for the TNP hapten were reacted with TNP-BSA and then binding of IgG to TNP-BSA was evaluated with HRP-conjugated anti-human IgG (FIG. 6A). The FcγR binding activities of the IgG:TNP-BSA immune complexes was measured with the following FcγR monomer biotin; hFcγRIIa His131 (FIG. 6B), hFcγRIIa Arg131 (FIG. 6C), hFcγRIIb (FIG. 6D), hFcγRIIIa Phe158 (FIG. 6E).

To examine higher avidity interactions, human and macaque FcγR monomers were complexed with streptavidin and then reacted with equivalent levels of TNP-BSA antigen:antibody immune complexes formed using recombinant IgG1, IgG2 and IgG4 (FIG. 6A). The streptavidin-complexed FcγRIIa His131 biotin preferentially bound IgG2 when compared to the Arg131 form of the protein (FIG. 6B, C, and Table 2). The streptavidin complexed FcγRIIb biotin equally bound IgG1 and IgG4 in accordance with the unique specificity of this receptor (FIG. 6D). The streptavidin complexed FcγRIIIa binding hierarchy IgG1>IgG4>IgG2 was as found with the dimeric version of the receptor excepting that the streptavidin:receptor complexes (FIG. 6C, D) had greater binding activity for the low activity IgG4 and IgG2 subclasses than did the receptor dimers. This again demonstrates that higher avidity interactions enhance the low affinity interactions to, potentially, even diminish the binding differences of Fc receptors to the different IgG subclasses. Similarly to the results shown in FIG. 5, the comparative use of FcγR dimers and FcγR oligomers can discriminate between IgG subclass capacity to bind FcγR at low and high levels of antibody.

Dimeric rsFcγRIIb Selectively Bind IgG Subclass ICs

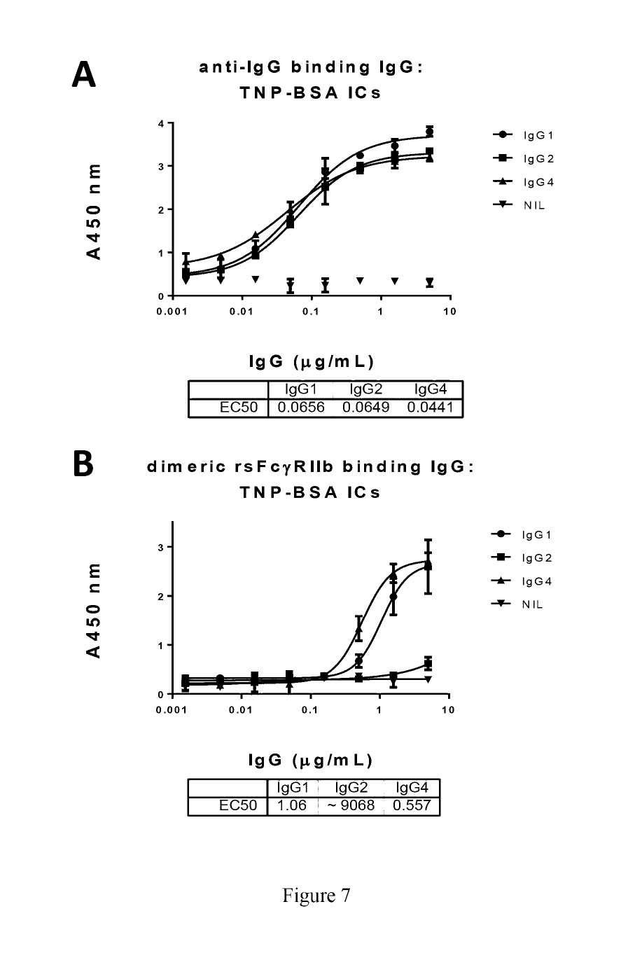

ELISA analysis was conducted using anti-TNP monoclonal antibodies of different human subclasses and TNP BSA coated on ELISA plates as described for the experiments shown in FIG. 3. The extracellular region or ectodomain of the inhibitory receptor FcγRIIb is highly related to the ectodomains of the activating receptors FcγRIIa and FcγRIII. Biotinylated dimeric rsFcγRIIb was produced as a single polypeptide and its interaction with human IgG subclasses analysed by ELISA (FIG. 7). In these experiments equivalent levels of opsonisation of TNP:BSA with the IgG1, IgG2, IgG4, was revealed by using HRP conjugated anti IgG (FIG. 7). However, clear differences in the interaction of the dimeric FcγRIIb were apparent and the EC 50 values indicate a hierarchy of binding of IgG4>IgG1>>>IgG2 FIG. 7B). This hierarchy is consistent with the binding of immune complexes to cell surface expressed FcγRIIb (Hogarth and Pietersz 2012).

FcγRIIIa Dimers Selectively Bind Non-Fucosylated Anti-CD20 Antibodies

The anti-CD20 mAb rituximab or the afucosylated anti-CD20 mAb obinutuzumab were captured using F(ab′)₂ anti-human F(ab′)₂, reacted with HRP-conjugated anti-human IgG (FIG. 8A) or with the following FcγR dimers: biotinylated FcγRIIIa Val158 dimer (FIG. 8B) or biotinylated FcγRIIIa Phe158 dimer (FIG. 8C) followed by detection with streptavidin-HRP. In addition to IgG subclass and presentation, glycosylation of the IgG Fc influences its interaction with FcγRs. Of interest, the absence of a bisecting fucosyl residue in the N-linked carbohydrate in the Fc of IgG1 enhances its binding to FcγRIIIa and antibody-dependent cell-mediated cytotoxicity (ADCC). This property is demonstrated by the biotinylated FcγRIIIa Val158 dimer which showed a modest (approximately 2-fold) enhanced level of binding to afucosylated obinutuzumab captured as an immune complex by the F(ab), anti-human F(ab′)₂ (FIG. 8B). In contrast, the FcγRIIIa Phe158 dimer had a strong binding preference for obinutuzumab (approximately 20-fold) over rituximab (FIG. 8C). Accordingly, FcγRIIIa dimers discriminate between antibodies with modified functions, discriminating between normally glycosylated and non-fucosylated forms of IgG1.

FcγRIIIa-Phe158 Biotin as Streptavidin Complexed Monomers or Dimers Selectively Bind Non-Fucosylated Anti-CD20 Antibodies

The anti-CD20 mAb rituximab or the afucosylated anti-CD20 mAb obinutuzumab at concentrations from 1000 to 10 ng/ml were formed into an immune complex by capture with plate bound F(ab′)₂ anti-human F(ab′)₂. These immune complexes were then reacted with streptavidin-HRP complexed FcγRIIIa Phe158 monomer biotin or biotinylated FcγRIIIa Phe158 dimer followed by detection with streptavidin-HRP. For the 1000 and 315 ng/ml antibody concentrations, the ratio of the FcR binding activity of obinutuzumab and rituximab was calculated. OD (A450 mn) values<0.1 were excluded from calculating this ratio and negated the evaluation of the results from the monoclonal antibody (mAb) concentrations <315 ng/ml, although preferential FcR binding to obinutuzumab was clearly more marked at <100 ng/ml (data not shown).

The streptavidin complexed FcγRIIIa-Phe158 monomer was compared with the dimeric form of this receptor in the binding of afucosylated IgG1 (obinutuzumab) compared to the unengineered glycosylated IgG1(rituximab). Both the FcγRIIIa Phe158 receptor ectodomains as streptavidin complexed monomers or as a genetic dimer showed receptor dose dependent and antibody dose dependent binding to IgG1 and enhanced binding to afucosyl-IgG1. Indeed both the streptavidin complexed FcγRIIIa Phe158 monomer (c.f. FIG. 8E with 8D) and the dimeric form of this receptor (c.f. FIG. 8G with 8F) showed preferential binding to the afucosylated IgG1 (obinutuzumab) compared to the unengineered glycosylated IgG1(rituximab). This enhanced binding to obinituzumab was most pronounced at the lower antibody (100 and 31 ng/ml) and low receptor (<25 ng/ml) concentrations (FIG. 8E, G). A more substantial binding preference for obinutuzumab over rituximab was displayed by the FcγRIIIa Phe158 dimers (FIG. 8F,G) than the streptavidin complexed FcγRIIIa Phe158 monomer (FIG. 8E, 8D). Again, this was greatest at the lower antibody (100 and 31 ng/ml) and low receptor (<25 ng/ml) concentrations.

The preference of binding to obinutuzumab or rituximab, at 1000 and 315 ng/ml IgG, clearly demonstrated the preferential binding of the FcγRIIIa Phe158 dimer to the afucosylated antibody. This preference was inversely correlated until the receptor and antibody concentration reached a 10-fold binding preference for obinutuzumab over rituximab (FIG. 8H). Thus, low levels of opsonising obinutuzumab more effectively binds FcγRIIIa dimer than equivalent levels of rituximab, which, while not wishing to be bound by theory, is considered to underlie the mechanistic basis of the greater ADCC activity of obinutuzumab, and other non-fucosylated anti-CD20 IgG molecules, over their normally glycosylated counterparts. FcγRIIIa dimers discriminate between antibodies with modified functions (ie between normally glycosylated and non-fucosylated forms of IgG1); this is most apparent with the FcγRIIIa Phe158 allotype and at low concentrations of ligand (IgG) and receptor dimer or oligomer. This discrimination is also more effective with the FcγRIIIa dimer than oligomers.

Streptavidin Complexed FcγRII Binds Equivalently to Fucosylated and Afucsoylated Anti-CD20 Antibodies

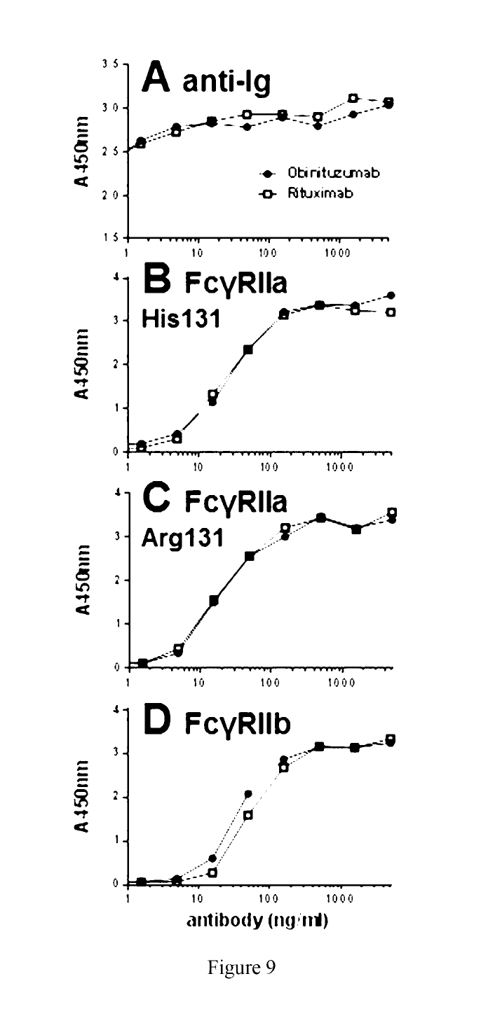

The anti-CD20 mAb rituximab or the afucosylated anti-CD20 mAb obinutuzumab were captured using F(ab′)₂ anti-human F(ab′)₂ and reacted with biotin-conjugated anti-human IgG (FIG. 9A) or with the following streptavidin (SA)-complexed FcγR biotin monomers: FcγRIIa His131 (FIG. 9B), FcγRIIa Arg131 (FIG. 9C) or FcγRIIb (FIG. 9D). In contrast to FcγRIIIa, the various forms of streptavidin complexed FcγRII biotin monomers, FcγRIIa His131, FcγRIIa Arg131 and FcγRIIb, showed near equivalent binding to obinutuzumab and rituximab immune complexes (FIG. 9). This concurs with reports that fucosylation of the Fc N-linked carbohydrate has little effect on FcγRII binding. Accordingly, the results indicate that unlike FcγRIIIa, FcγRIIa does not distinguish between fucosylated and non-fucosylated IgG. Thus, the prediction of FcR activity is not surprising and the assay is useful for discriminating between IgG molecules with characteristics that alter FcγRIII binding activity and those that alter FcγRII binding activity.

FcγRIIIa-Phe158 Biotin Dimer Selectively Binds Afucsoylated Anti-CD20 Antibody Opsonised Daudi Cells

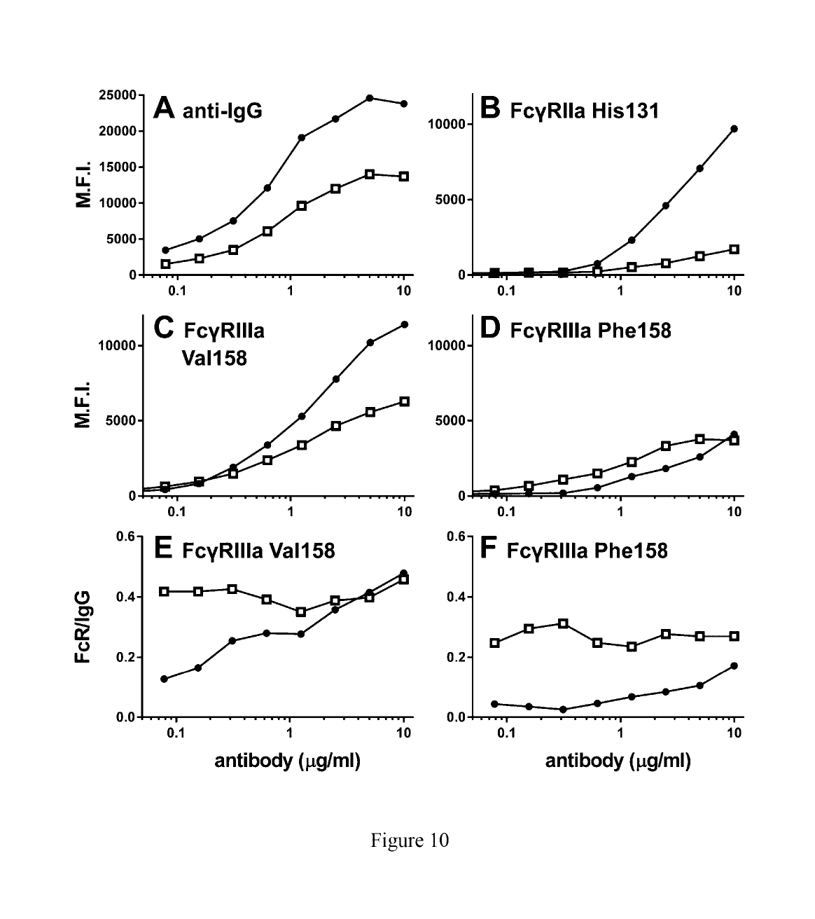

Daudi cells were reacted with the anti-CD20 mAbs rituximab or obinutuzumab at concentrations from 10 μg/ml to 0.1 μg/ml, and the opsonised cells were then reacted with FITC-conjugated anti-human IgG Fc (FIG. 10A) or with the following FcγR dimers: biotinylated FcγRIIa-His131 dimer (FIG. 10B), biotinylated FcγRIIIa-Val158 dimer (FIG. 10C) or biotinylated FcγRIIIa-Phe158 dimer (FIG. 10D). Specific activities of binding (ie FcR binding normalised for IgG binding) were calculated for each antibody concentration as a relative measure of binding stoichiometry for the two allelic forms of FcγRIIIa, that is, FcγRIIIa-Val158 dimer (ie MFI values panel C/values panel A; FIG. 10E) and FcγRIIIa-Phe158 dimer (ie MFI values panel D/values panel A; FIG. 10F). Representative histograms derived from the flow cytometry data from FIG. 10 of the Daudi cells opsonised at 2.5 μg/ml rituximab or obinituzumab were generated for anti-human IgG, FcγRIIa-His 131, FcγRIIIa-Val158 and FcγRIIIa-Phe158 (data not shown). Taken together, the data demonstrated that the binding of Fc receptor dimers or multimers complexes with streptavidin was characterised by IgG subclass, Fc presentation and Fc glycosylation. However, to evaluate the potential ADCP and ADCC activities of IgG antibodies, such as anti-CD20 antibodies, binding to cellular targets was assessed.

Daudi cells were found to be opsonised by rituximab to twice the level of obinutuzumab, consistent with these being type I and type II anti-CD20 mAbs, respectively, whereby type I mAbs (eg rituximab) have a 2-fold higher stoichiometry for CD20 than type II mAbs (FIG. 10A). Consequently, obinutuzumab opsonised cells weakly bound the biotinylated FcγRIIa His131 dimer compared to rituximab (FIG. 10B). Likewise, at the highest antibody concentration (10 μg/ml), the biotinylated FcγRIIIa Val158 dimer bound to rituximab opsonised cells at approximately twice the level of the obinutuzumab opsonised cells FIG. 10C; whereas at low antibody concentration (<0.3 μg/ml), binding to the two antibodies was equivalent.

In contrast, while the biotinylated FcγRIIIa Phe158 dimer had less binding activity than the Val158 protein, the Phe158 dimer preferentially bound the obinutuzumab opsonised cells over the rituximab opsonised cells despite the higher levels of opsonisation by rituximab (FIG. 10D). Accordingly, despite lesser binding of the afucosylated obinutuzumab antibody than rituximab to the cells, the FcγRIIIa dimer preferentially bound the obinutuzumab opsonised cells and this was most apparent for the FcγRIIIa-Phe158 dimer receptor. When the binding of receptor is expressed as a ratio over the amount of bound antibody, it is apparent that the afucosylated mAb has near unchanged, concentration-independent, binding of both the FcγRIIIa-Val158 and Phe158 dimers over a hundred-fold concentration range (0.1 to 10 μl/ml). That is, at low levels of opsonisation achieved at input concentrations of approximately 0.1 μg/ml anti-CD20 mAb, the amount of receptor engaged per antibody (relative stoichiometry) is the same as that found at high opsonisation achieved at an input concentration of approximately 10 μg/ml. The FcR binding behaviour of rituximab, in contrast, shows a concentration dependence with the relative stoichiometry of FcR/IgG for the Val158 dimer being low at 0.1 μg/ml input concentration but increasing by 5-fold at 10 μg/ml (FIG. 10E). The Phe158 allele likewise shows a concentration dependence of FcR/IgG activity; however this dependence is less in slope and magnitude, as is consistent with being the lower affinity allelic receptor. (FIG. 10F). Together, these data are consistent with the afucosylated obinutuzumab's superiority to rituximab for engaging both FcγRIIIa-Val158 and Phe158 receptors when targets are sparsely opsonised with antibody.

Accordingly, in a FACS-based model of cancer cell detection and mAb therapy, antibodies modified for effector function (ie afucosyl IgG with enhanced ADCC activity) can be distinguished, with FcγRIIIa-Val158 and more effectively, FcγRIIIa-Phe158 dimer, selectively detecting the ADCC-enhanced antibody. Despite higher levels of opsonisation by rituximab, the FcγRIIIa-Phe158 dimer specific activity is greater for the afucosyl anti-CD20 obinituzumab.

FcγRIIa Binding to Opsoninised Daudi Cells Reflects the Lower Level of Bound Obinutuzumab (Type II mAb) Compared to Rituximab (Type I mAb)

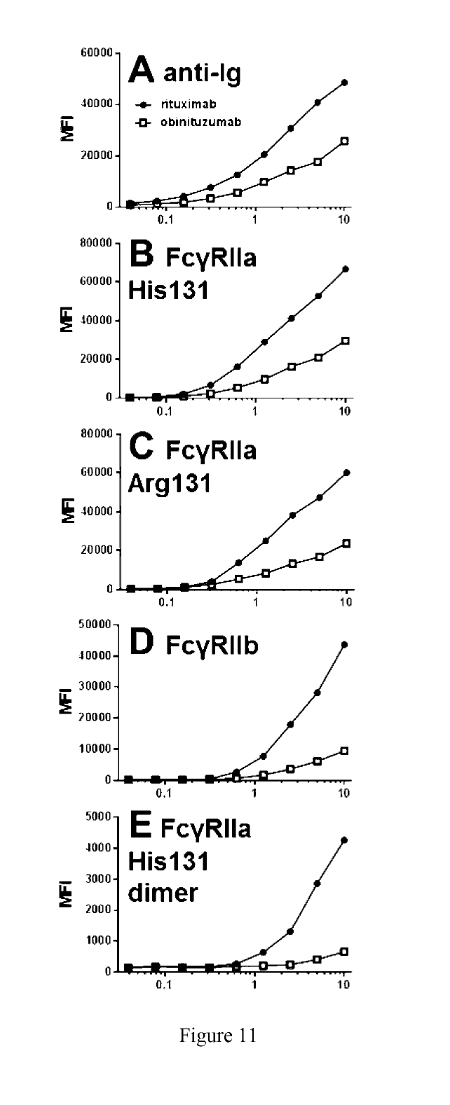

Daudi cells were reacted with the anti-CD20 mAbs rituximab or obinutuzumab at concentrations from 10 μg/ml to 0.1 μg/ml and the opsonised cells were then reacted with FITC-conjugated anti-human IgG Fc (FIG. 11A) or with the following streptavidin complexed FcγRII monomers: biotinylated FcγRIIa His131 (FIG. 11B), biotinylated FcγRIIa Arg131 (FIG. 11C), biotinylated FcγRIIb (FIG. 11D) or with biotinylated FcγRIIa His131 dimer (FIG. 11E). Daudi cells were opsonised to twice the level by rituximab compared to the mAb obinutuzumab, consistent with these being type I and type II anti-CD20 mAbs, respectively. The level of binding of all forms of FcγRII (ie FcγRIIa-His131 and Arg131 allelic receptors) as dimer or SA-complexed monomer forms, and FcγRIIb was greater for rituximab opsonised cells and thus correlated with the high (type I) and low (type II) levels of mAb opsonisation (FIG. 11). Accordingly, the different forms of FcγRII do not discriminate between normally fucosylated and non-fucosylated IgG, when formatted either as streptavidin oligomerised complexes or as a dimer.

Flow Cytometric Analysis of Cell Opsonisation by Therapeutic Antibodies in Peripheral Blood B Lymphocytes Cells

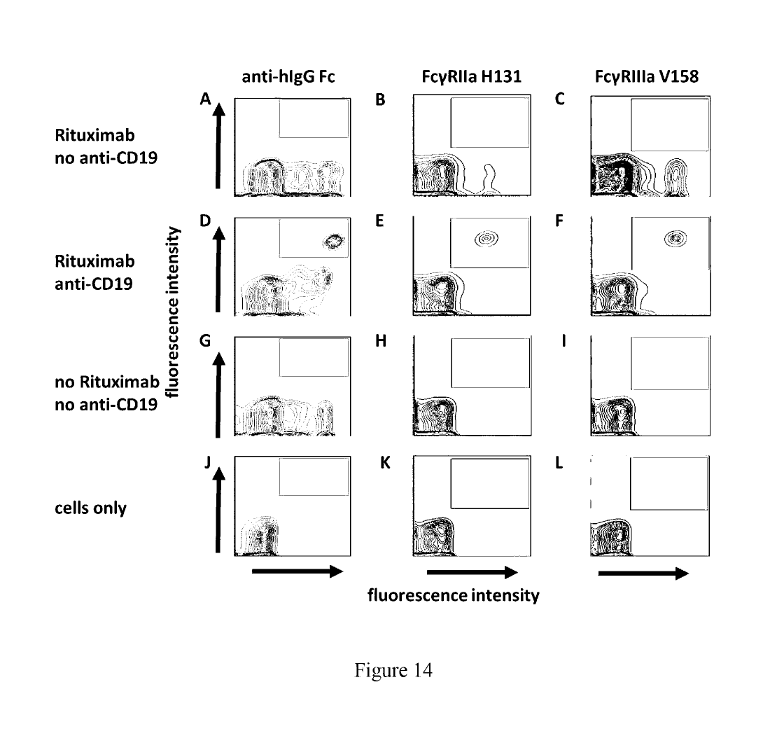

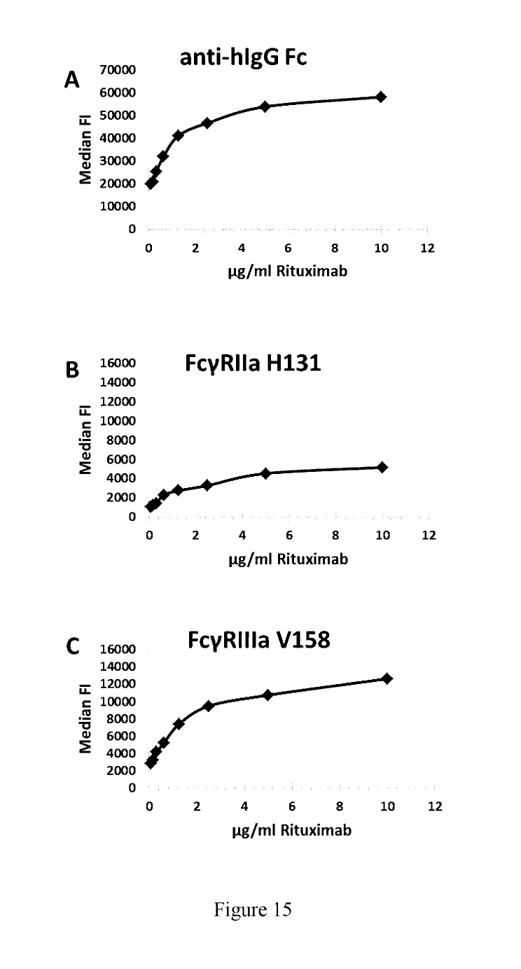

The use of the FcR multimers to detect properties of antibodies was extended to the analysis of peripheral blood cells and B lymphocytes in particular (FIG. 14 and FIG. 15). Peripheral blood mononuclear cells were reacted with the anti-CD20 mAb rituximab (2.5 μg/ml) PBMC opsonised with 2.5 μg/ml rituximab (FIG. 14A-F) or diluent, FACS buffer, only (FIG. 14G-L). Staining with an anti-human IgG Fc to determine opsonisation of the cells by rituximab demonstrated substantial cell opsonisation (FIG. 14A, D) compared to anti IgG Fc staining of un-opsonised cells (FIG. 14G). Indeed, a substantial population of rituximab opsonized cells were CD19-positive B lymphocytes (FIG. 14D). Interactions with rituximab opsonized cells were apparent for the binding of dimer FcγRIIa H131, (FIG. 103B, E,) and also the dimer FcγRIIIa-Val158 (FIG. 14C, F). The receptor dimers were used at 0.2 μg/ml and there was no binding of either of the FcγR dimers to unopsonised cells (FIG. 14 H, I, K, L) demonstrating that the observed FcR binding to opsonised cells was dependent on the presence of the anti CD20 mAb.

Since B cells express CD20 and are detected by anti CD20 mAbs including rituximab (Klein et al., 2013), the lineage marker, CD19 was used to identify B cells in the PBMC (FIG. 14 D, E, F). It is apparent that the CD19 B cells opsonised with the rituximab bound to the FcγRIIa-His131 (FIG. 14 E) and to FcγRIIIa-Val158 (FIG. 14 F). However, distinct differences were apparent in the strength of binding wherein FcγRIIa-His131 showed lower median fluorescence intensity (MFI) of approximately 4000 being less than half of the binding of FcγRIIIa-Val158 (MFI approximately 11000) (FIG. 14 F). The differences in binding of opsonised peripheral blood B cells to FcγRIIa-His131 FcR dimer and FcγRIIIa-Val158 FcR dimer were apparent over concentrations of rituximab from 10 μg/ml to 0.1 μg/ml (FIG. 15). The opsonised CD19-positive cells reacted with FITC-conjugated anti-human IgG showed the extent of opsonisation over the range of antibody concentrations (FIG. 15 A). Titration of the FcγRIIa-His131 (FIG. 15 B) revealed lower binding at every concentration than that obtained with FcγRIIIa-Val158 (FIG. 15 C). Thus, the FcγRIIa-His131 has a lower affinity interaction. This indicates that the distinct FcR multimers, FcγRIIa-His131 and FcγRIIIa-Val158, can interact differently with the same opsonised cells which is consistent with their different affinities and functional capabilities on cells (Hogarth and Pietersz, 2012).

Characterisation of Normal and Variant Anti-HIV Env mAb b12 with rsFcγR Assay

Therapeutic use of mAbs often uses variants of the Fc which commonly include enhanced reactivity for FcγR or abrogated reactivity for FcγR. One important enhanced variant of the Fc involves the afucosyl-glycosylation form and an Fc receptor binding reduced form is the Leu, Leu (234,235)Ala, Ala mutant (aka LALA) IgG. Subsequently, the ability of the FcR assays to detect the enhanced or reduced activities of these variant antibodies was examined.

Normal mAb b12, an unmodified human IgG1 monoclonal antibody (afucosyl-b12 a variant of mAb b12 that lacks fucose on the N-linked oligosaccharide of the IgG1 H chain), and LALA mutant b12 (a mutant derived form mAb b12 that has been mutated to replace the lower hinge residues Leu234 Leu235 with Ala234 and Ala235), were captured using F(ab′)₂ anti-human F(ab′), to form immune complexes and reacted with HRP-conjugated anti-human IgG (FIG. 12A) or the following human FcγRs (as streptavidin oligomerised monomers, or when indicated as dimers); streptavidin oligomerised monomer FcγRIIa-Arg131 (FIG. 12B), streptavidin oligomerised monomer FcγRIIa-His131 (FIG. 12C), dimer FcγRIIa-His131 (FIG. 12D), streptavidin oligomerised monomer FcγRIIb (FIG. 12E), dimer FcγRIIIa-Val158 (FIG. 12F), dimer FcγRIIIa-Phe158 (FIG. 12G), and streptavidin oligomerised monomer FcγRIIIa-Phe158 (FIG. 12H).

The results indicate that the FcγR assays are useful for discriminating both enhancing and diminishing variants of IgG. For example, afucosyl b12 was observed to have higher activity for FcγRIIIa compared to normal mAb b12, and is unchanged for FcγRII binding compared to normal b12 mAB; whereas the LALA mutant antibody, which has a mutation in the IgG lower hinge, shows reduced binding activity compared to normal mAb b12. Notably, the streptavidin oligomerised monomers of FcγRIIa (FIGS. 12 B and C), FcγRIIb (FIG. 12 E) and FcγRIIIa-Phe158 (FIG. 12 H) have substantial binding activity to the supposed FcγR abrogating LALA mutation. This demonstrates that some FcγR inhibiting mutations may have residual FcγR binding activity that can lead to substantial binding when the avidity of the interaction is sufficiently high (for example, in large immune complexes, or heavily opsonised cell targets, of sufficient receptor expression on the effector cell).

2G12 is the prototype of IgGs with non-covalently fused Fab regions. The I19R variant is a mutation towards the gem line sequence which disrupts the VH:VH domain interface which dissociates the non-covalently joined Fab regions to form two distinct Fabs as is found in every other described IgG antibody. The FcR dimer assay was tested with this antibody, as an IgG1, an IgG3 and as an I19R revertant to test if the assay can distinguish the FcR activities of these antibodies with different topologies.

Characterisation of Normal and Variant Anti-HIV Env mAb 2G12 with rsFcγR Assay

The FcγR assay was tested using an anti-HIV-1 gp120 IgG1 monoclonal antibody 2G12 and variants thereof; specifically, wild type mAb 2G12-IgG1 having a domain-swapped VH regions which result in Fab regions being atypically fused into a single binding domain (circles; obtained from and as described in Huber, 2010; a wild type 2G12 formatted as an IgG3 (squares a GeneArt (Invitrogen) codon-optimised sequence corresponding to accession AK126133.1); a I19R revertant mutant formatted as an IgG3 having structurally typical IgG topology (ie separate Fab regions) (triangles; a GeneArt (Invitrogen) codon-optimised sequence corresponding to accession AK126133.1).

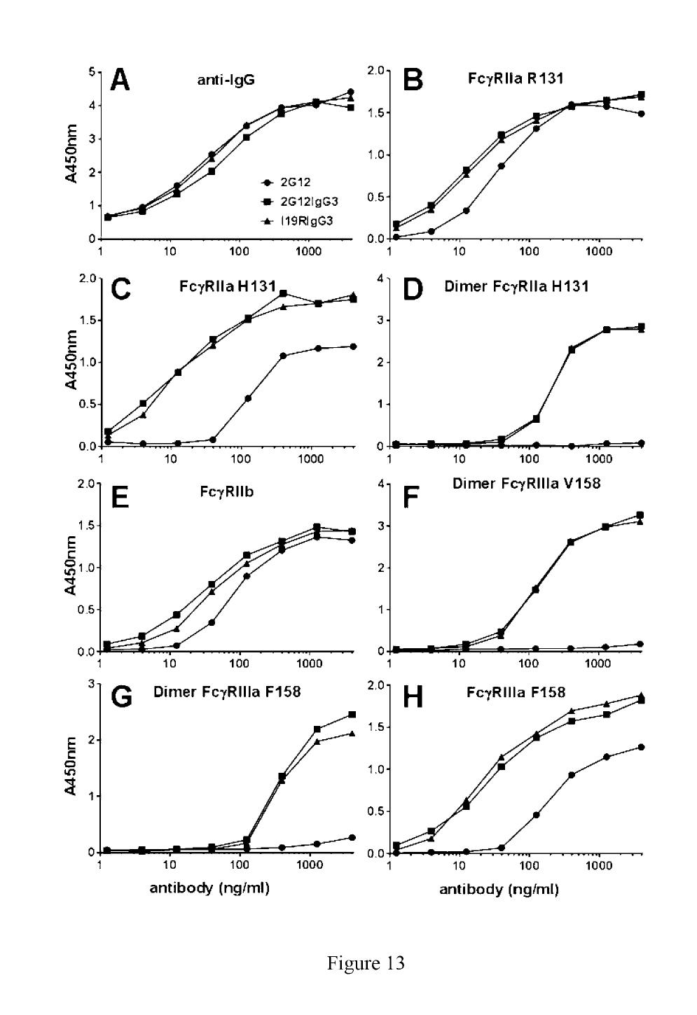

The antibodies were captured using F(ab′)₂ anti-human F(ab′)₂ to form immune complexes and reacted with HRP-conjugated anti-human IgG (FIG. 13A) or the following human FcγRs as streptavidin oligomerised monomers, or when indicated, as dimers: streptavidin oligomerised monomer FcγRIIa-Arg131 (FIG. 13B), streptavidin oligomerised monomer FcγRIIa His131 (FIG. 13C), dimer FcγRIIa-His131 (FIG. 13D), streptavidin oligomerised monomer FcγRIIb (FIG. 13E), dimer FcγRIIIa-Val158 (FIG. 13F), dimer FcγRIIIa-Phe158 (FIG. 13G), and streptavidin oligomerised monomer FcγRIIIa-Phe158 (FIG. 13H). The results indicate that the FcγR assays are useful for discriminating antibodies with normal topology for IgG (ie 2G12 I19R variant) and the unusual fused Fab form of WT 2G12 in which the Fab regions are non-covalently fused (2G12-IgG1 and 2G12-IgG3). Notably, compared to a structurally typical IgG (2G12 I19R-IgG3), the WT 2G12-IgG1 has diminished binding to FcγRIIa-His131 (FIGS. 13C and D) and FcγRIIIa (FIGS. 13F, G and H) but higher activity for FcγRIIa-Arg131 (FIG. 13B) and FcγRIIb (FIG. 13E). The results indicate that the diminished activity for FcγRIIa-His131 and for FcγRIIIa is lessened when the receptors were formatted as high avidity streptavidin complexed oligomers. Additionally, the structurally atypical form of 2G12 formatted as a IgG3 antibody (ie 2G12-IgG3) had a “no′ mat” high activity on all the FcR tested, unlike the 2G12-IgG1. This suggests that the fused Fab regions of the wild type 2G12 may place steric constraints on FcγR interactions, a constraint which is relieved in the context of the long hinge of IgG3 of 2G12-IgG3.

Soluble FcR Distinguish Antibody Responses in Individual HIV Patients

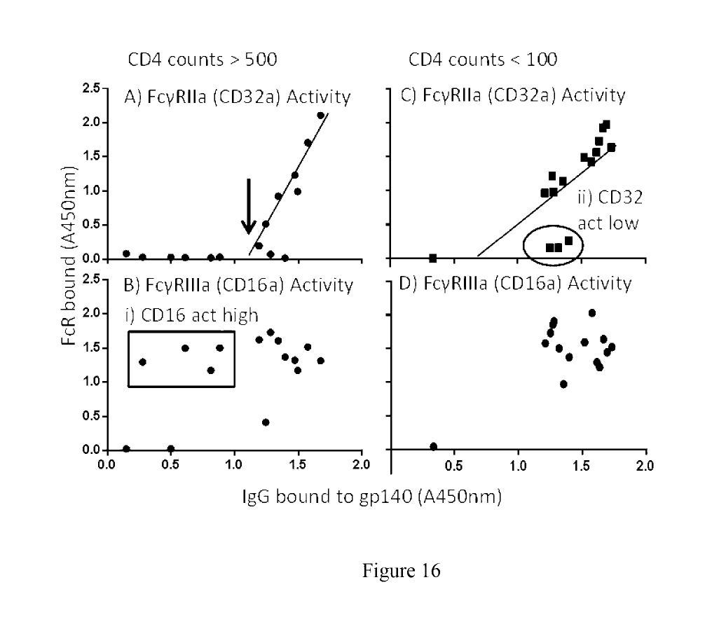

The FcγR dimers were used in an ELISA assay to test antibody responses to env protein in HIV patients (FIG. 16). The sera of 30 patients; 15 patients with high CD4 counts (>500 cells/μl) and 15 patients with low CD4 counts (<100 cells/μl) were reacted with recombinant gp140 HIV AD8 envelope trimer (Center et al., 2009). FIG. 16A demonstrates that below a certain level of bound opsonising IgG antibody, in this experiment an OD (A450 nm) of ˜1.0, there is essentially no binding of the FcγRIIa dimer. The arrow denotes this threshold of IgG opsonisation necessary for FcγRIIa (also referred to as CD32a) binding. As can be seen in FIG. 16 B, it was demonstrated that some patients have substantial FcγRIIIa (also referred to as CD16a) dimer binding activity while the level of bound IgG is comparatively low. This patient subpopulation is indicated by the box which encloses data from sera with high CD16a binding activity. FIG. 16 C demonstrates that some patient sera have low FcγRIIa dimer binding activity compared to others with equivalent levels of bound IgG. This patient subpopulation is indicated by the oval which encloses data from sera with low FcγRIIa (CD32a) binding activity. In FIG. 16 D, it can be seen that with the sera from patients with CD4 T cells <100/μl most have relatively high levels of anti-env IgG that form one population that is not stratified into subpopulations on the basis of FcγRIIIa dimer activity.

Discussion—

The lower affinity FcγR (eg FcγRII and FcγRIII) can induce ADCC and ADCP by immune system effector cells following multivalent binding of antigen specific IgG in immune complexes formed on the surface of a target cell or pathogen. Advantageously, a probe for evaluating the activity of IgG immune complexes may mimic the cell surface clustering of FcγRs by these ligands.

In the present disclosure, FcR multimers (probes) were generated that comprised biotin-tagged monomeric FcγR ectodomains oligomerised by reaction with streptavidin to form multimers, or which otherwise comprised genetically fused dimeric FcγR that were biotin tagged. These FcγR multimers bound immune complexes formed on plastic surfaces or cell surfaces and demonstrated hierarchies of binding of the different IgG subclasses similar to that reported for cell surface receptors. Further, the multimers comprising FcγRIIb or allelic forms of FcγRIIa, such as the FcγRIIa His/Arg131 polymorphs, showed their expected distinctive patterns of binding, distinguishing the lower abundance subclasses, IgG2 and IgG4. The genetically fused dimers showed greater binding differences to different forms of IgG than did the corresponding biotin-streptavidin induced oligomers. Further, afucosylated IgG1, as exemplified by the therapeutic antibody obinutuzumab, was more potent in binding FcγRIIIa dimers and biotin-streptavidin induced oligomers than the normally glycosylated rituximab. Similarly, the afucosyl form of the anti-HIV gp120 mAb b12 was more active in binding FcγRIIIa than the normally glycosylated mAb. Accordingly, the properties of these FcγR multimers are capable of distinguishing the immune activating capacities of different IgG antibody mutants, subclasses and glycoforms.

FcγR dependent cellular functions are a necessary arm of the effectiveness of a number of anti-tumour antibodies and may also be responsible for some deleterious effects of therapeutic antibodies. The efficacy of engagement of FcγRs depends on the intrinsic properties of the Fc domain such as particular amino acid residues at the interaction interfaces and glycosylation, but also depends on the features utilised by an antibody to form an immune complex. Such features include antibody topology, orientation, antibody density, and steric effects such as epitope proximity to a cell membrane. While the intrinsic properties of the Fc domain can be engineered, binding is influenced by the approximately 10¹¹ potential antibody:antigen interactions (Glanville 2009) responsible for target opsonisation and is not easily predicted.

FcγRII and FcγRIII expressing leukocytes selectively bind IgG immune complexes by the avid interactions of multiple Fcs with the Fc receptors that cluster on the cell surface. Fc receptor clustering results in signalling and cellular responses that underlie the efficacy of many therapeutic antibodies. The present applicants mimicked this interaction in a FcR binding assay which can be used to evaluate the binding of Fc receptors, such as Fc receptor monomer-biotin/streptavidin HRP complexes or genetically fused dimer-biotin receptor ectodomains with various antibodies or parts thereof. Complexing receptor monomer-biotin in excess with streptavidin-HRP can result in saturation of biotin binding sites of streptavidin, and the low affinity of the receptor monomers ensures they are poor inhibitors of the receptor/streptavidin complexes.

The strength of binding of antibody immune complexes, as measured by the assay of the present disclosure, was determined by: 1) antibody subclass, 2) species differences, 3) antibody density, 4) receptor allotype, 5) antibody presentation/topology, and/or 6) Fc fucosylation. Additionally, the assay showed “low activity” interactions, such as IgG4 binding to FcγRIIa can bind strongly when the immune complex is of sufficient avidity. This may occur with some cellular antigens which may reach high local concentration on the cell surface. Fc receptor interaction is key to the adverse activity of the TGN412 IgG4 formatted anti-CD28 antibody highlighting the benefit of testing these interactions under a range of receptor/antibody avidities. The assay also indicated the superiority of afucosyl-anti-CD20 Ab over a range of antibody concentrations in targeting a cell for both FcγRIIIa Val and Phe158 interactions.

### Example 2 Dimeric rsFcγRIIa Binding is Dependent on the Density of Antibody Opsonisation

Materials and Methods—

TNP-BSA was produced by reacting BSA (10 mg/ml) with 1.8, 1.4, 0.9, 0.6 mM 2,4,6-trinitrobenzenesulfonic acid, dialysed extensively against PBS and the level of modification determined by absorbance at 280 and 340 nm. Human anti-TNP IgG1 at concentrations from 500 to 1 ng/ml were formed into ICs by binding to TNP-BSA comprising 1.9, 3.3, 4.2 or 5.1 TNP molecules per BSA molecule. These ICs were then reacted with HRP-conjugated anti-human IgG1 or with dimeric rsFcγRIIa H131 with subsequent detection with streptavidin-HRP. Binding data (mean±SD, n=2) were fitted using Prism software, to log (agonist) vs. response (variable slope, constraining bottom value=0, and EC50 shared, the top value was allow to vary freely). The top value of the binding curves (i.e. Bmax) for anti-IgG and dimeric rsFcγRIIa were plotted against stoichiometry of TNP modification.

### Results and Discussion

Results and Discussion—

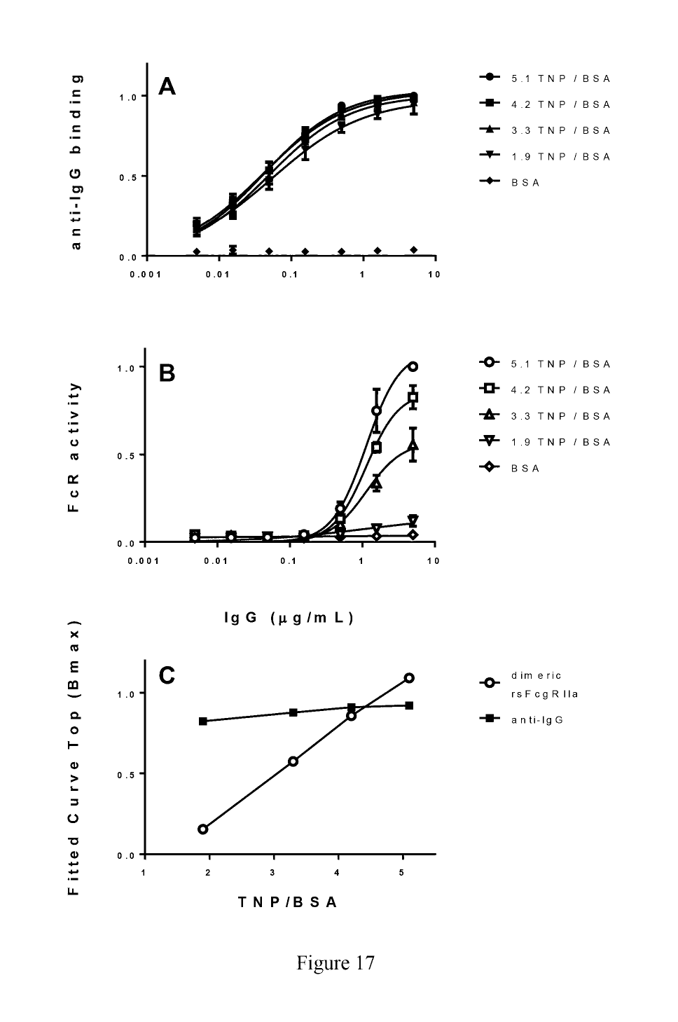

The capacity of dimeric FcγR to probe immune complexes formed at different antibody densities was tested using TNP BSA wherein the BSA contained varying numbers of TNP residues, on average 5.1, 4.2, 3.3, and 1.9 residues per BSA molecule. FIG. 17 shows a reduction in the antigen/opsonisation density influenced the binding of the dimeric RS FcγRIIa H131 to human IgG1. For IgG binding (FIGS. 17 A and C), there is a relatively shallow decrease in maximum binding levels (i.e. total binding sites or Bmax) as the level of TNPs per BSA decreases, that is, the signal generated by the anti-IgG HRP conjugate is relatively insensitive to differing opsonisation of the different TNP modified-BSA antigens with anti-TNP IgG1. In contrast, Bmax decreases steeply with the level of TNP-haptenylation for dimeric rsFcγRIIa H131 binding. This is consistent with the dimeric rsFcγR binding to pairs of closely spaced IgG molecules, the number of which decreases with the level of TNP modification of the BSA in the ICs.

### Example 3 Dimeric rsFcγRIIIa Val158 Assay Correlates with NK-92-FcγRIIIa Val158 Activation by Influenza a HA, A/Perth/16/2009 (H3N2)

Materials and Methods—

NK cell activation was measured by HA:anti-HA IC dependent induction of intracellular IFNγ and cell surface CD107a. The NK cell line NK-92 (55) expressing human FcγRIIIa Val158 (GFP-CD16 (176V) NK-92) was used in the NK cell activation assays and was kindly provided by Dr. Kerry Campbell at the Institute for Cancer Research in Philadelphia, Pa. Briefly, 96-well ELISA plates (Nunc, Rochester, N.Y.) were coated with 600 ng of purified HA protein overnight at 4° C. in PBS. Following PBS washing, a blocking step was then performed with PBS containing 5% BSA (Sigma Aldrich) and 0.1% Tween 20 (U-CyTech) for 2 h at 37° C. Once blocked, plates were washed with PBS and incubated with heat-inactivated sera/plasma or Mg for 2 h at 37° C. Plates were washed with PBS then 2×10⁵ GFP-CD16 (176V) NK-92 cells were added to each well and incubated at 37° C. with 5% CO2 for 5 h. Anti-human CD107a allophycocyanin (clone A4H3; BD Biosciences) and 1 nM EDTA were added to the cells for 30 min at room temperature in the dark. GFP-CD16 (176V) NK-92 cells were then washed twice with PBS, fixed with 1% formaldehyde and acquired on the LSRII flow cytometer. Analysis was performed using FlowJo X 10.0.7r2 software (FlowJo LLC, Ashland, Oreg.).

ICs were formed by reacting plasma from 30 individuals with plate-bound HA A/Perth/16/2009 (H3N2). ICs formed with plasmas diluted at 1:80 were assessed for the binding of dimeric rsFcγRIIIa Val158 and compared with IC (formed with plasmas diluted at 1:40) for the capacity to activate NK-92 cells expressing FcγRIIIa Val158. FcγRIIIa Val158 binding was normalised using binding in wells directly coated with Mg (5 μg/4 ml). The two assays were correlated using non-parametric spearman analysis and fitted by linear regression.

Results and Discussion—

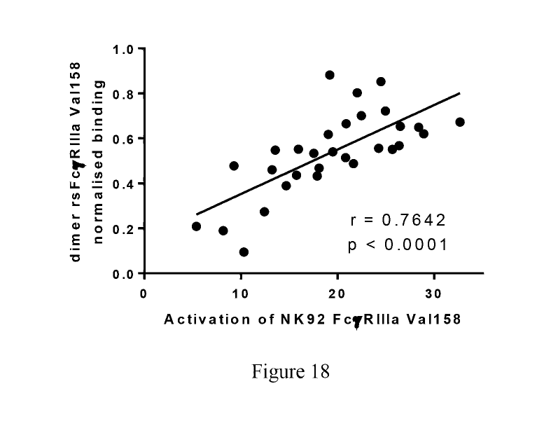

The relationship between dimeric rsFcR binding and cellular FcγR effector function was investigated. The ability of dimeric rsFcγR binding activity to correlate with cellular activation by HA-specific antibodies was tested by comparison with the activation of NK92 cells expressing FcγRIIIa Val158, a well-established system for measuring Ab-mediated NK activation. Plasma from individuals were used to separately opsonize HA Perth (H3N2) and used to activate NK92-FcγRIIIa (Val158) cells and for binding dimeric rsFcγRIIIa (Val158). The cell and dimeric rsFcγRIIIa activities correlated strongly (p<0.0001) validating use of the dimeric rsFcγRIIIa receptor to predict cellular responses (FIG. 18). Thus, the FcR dimer assay ranks individuals on the basis of the NK-cell activating potential of their anti-HA IgG antibodies.

### Example 4 Use of Dimeric Recombinant Soluble FcγR to Determine Proximity Mapping of Antibody and Epitopes

### Materials and Methods

Generation of IL12Rb1 Transfected Baf/3 Cells—

The IL-12Rβ1 transfectants were generated by retroviral gene transduction of BaF3 using IL-23R cDNAs amplified from IMAGE 8860351 (a frameshift mutation was repaired) and LIFESEQ95137702 cDNA clones with Pfx (Invitrogen, Carlsbad, Calif.) and subcloned into pENTR1A (Invitrogen) and then by LR clonase reaction inserted into the Gateway RfA cassette modified expression vectors pAPEX3p and pAPEX3 (Evans et al., 1995); and pMXIpuro and pMXIneo (Wines et al. 2004).

Proximity or Topology Mapping—

Mouse BaF3 cells expressing human IL-12Rβ1 were opsonised with individual anti-IL-12Rβ1 monoclonal antibodies (mAbs) or pairs of IL-12Rβ1 mAbs or a mixture of all 9 IL-12Rβ1 mAbs. The antibodies used are designated X81-1, X81-20A, X81-34, X81-39, X81-48, X81-80 X81-101, X81-109, X81-128. The X81 series of mAbs specific for the IL-12Rβ1 cytokine receptor subunit were generated by immunising mice with Ba/F3 cells transduced to express the IL-12Rβ1 and IL-23 cytokine receptor subunits. The generation of hybridomas producing the mAbs was as described in Ierino et al. 1993 and the selection of IL-12Rβ1 specific mAbs was determined by comparison of binding to parental Ba/F3 cells and Ba/F3 cells expressing the IL-12Rβ1 alone.

In experiments with single IL-12Rβ1 mAbs, 250,000 Mouse BaF3 cells expressing human IL-12Rβ1 in 50 μL of FACS buffer (1% BSA/phosphate buffered saline including 0.1% glucose) were incubated with 25 μL of undiluted supernatant from each IL-12Rβ1 hybridoma cell line; unopsonised control cells were incubated in FACS buffer only. The cells were the incubated for one hour on ice washed 3 times in FACS buffer then resuspended in 50 μL of biotin-tagged anti-mouse IgG (diluted 1/500) or 50 μL of dimeric recombinant soluble FcγRIIa R131 at 0.2 mcg/mL FACS buffer only. After 45 minutes incubation on ice cells were washed once and resuspended in 500 μL in FACS buffer and analysed by flow cytometry. For analysis of the mixture of all 9 mAbs, 50 ul of a mixture of equal volumes of undiluted hybridoma supernatants was added to 250,000 cells in 50 ul of FACS buffer and processed as for the single antibody analysis.

In the analysis of pairwise combinations of IL-12Rβ1 antibodies, 250,000 cells in 50 μL FACS buffer were incubated with 25 μL of undiluted supernatant from each of two IL-12Rβ1 hybridomas. The cell/antibody mixtures were incubated and process for flow cytometry as described for the single antibody analysis above. Detection of binding was performed using APC-conjugated streptavidin as above.

Results and Discussion—

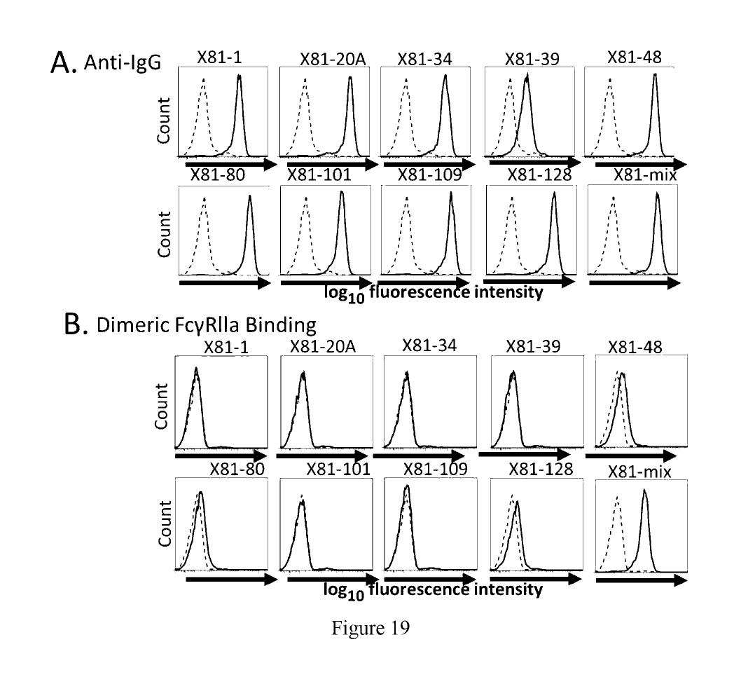

Flow cytometric determination of antibody proximity using dimeric FcγR binding to IL-12Rβ1 transfected BaF/3 cells opsonised with each of 9 monoclonal anti-IL-12Rβ1 antibodies X81-1, X81-20A, X81-34, X81-39, X81-48, X81-80, X81-101, X81-109, X81-128 or a mixture of all antibodies X81-mix. FIG. 19 A shows the extent of opsonisation of the cells by each monoclonal antibody or the mix of antibodies, determined by staining with a biotinylated anti-mouse IgG and APC-conjugated streptavidin (solid black line histograms), with background control staining of cells with anti IgG APC-streptavidin in the absence of IL-12Rβ1 monoclonal antibody (dashed line histograms). FIG. 19 B shows the binding of the biotinylated dimeric recombinant soluble FcγRIIa to IL-12Rβ1 opsonised cells detected with APC-conjugated streptavidin (solid black line) and the background control staining of opsonised cells with APC-conjugated streptavidin in the absence of FcγR dimer (dashed line). Dimeric FcγRIIa binding is most evident in the cells opsonised with a mixture of the IL-12Rβ1 mAbs.

This experiment evaluates Fc receptor binding of dimeric recombinant soluble Fc receptors on opsonised cells. In order to activate ADCC killing of an opsonised target, the Fc of the opsonising antibodies need to be sufficiently close to cluster Fc receptors into an activation complex, and this correlates with the binding of dimeric FcR to an opsonised target (FIG. 18, FIG. 19B). There is a clear relationship between the amount of dimeric FcR binding to the opsonised target (immune complex) and the capacity of that opsonised target to activate NK cells in an ADCC manner. This strong correlation between cell activation and dimeric recombinant soluble dimer binding, such as for FcγRIIIa, validates that the dimeric FcR described herein can predict cellular responses (FIG. 18). FIGS. 18 and 19, taken together indicate that other Fc multimers including the FcγRIIIa dimer can also be used in proximity mapping studies or for identifying pairs of therapeutic antibodies that would be useful for inducing immune cell effector function.

Since the binding of the dimeric recombinant soluble FcγR is dependent on topology or “near neighbour” proximity of antibody Fc regions it should be possible to determine the relationship between multiple antibodies bound to a target entity, for example, a whole cell, or a molecule on a cell, or different molecules on the one cell, or molecule or molecules in solution. To determine the utility of using the dimeric low affinity receptors as such a proximity probe and analysis of the relationship between nine monoclonal antibodies that detect a single molecule, the beta chain IL-12Rβ1 of the IL-12/IL-23 receptor complex. Cells were opsonised with individual IL-1Rβ1 monoclonal antibodies or a mixture of all nine antibodies. Despite equivalent levels of opsonisation with each of the individual antibodies (FIG. 19A), limited binding of dimeric FcγRII was detected (FIG. 19B). However, when the antibodies were mixed, clearly detectable binding of the dimeric FcγR was observed (FIG. 19B, labelled X81-mix) indicating that certain combinations of antibodies were in sufficiently close proximity to be permissive of dimeric FcγRII binding.

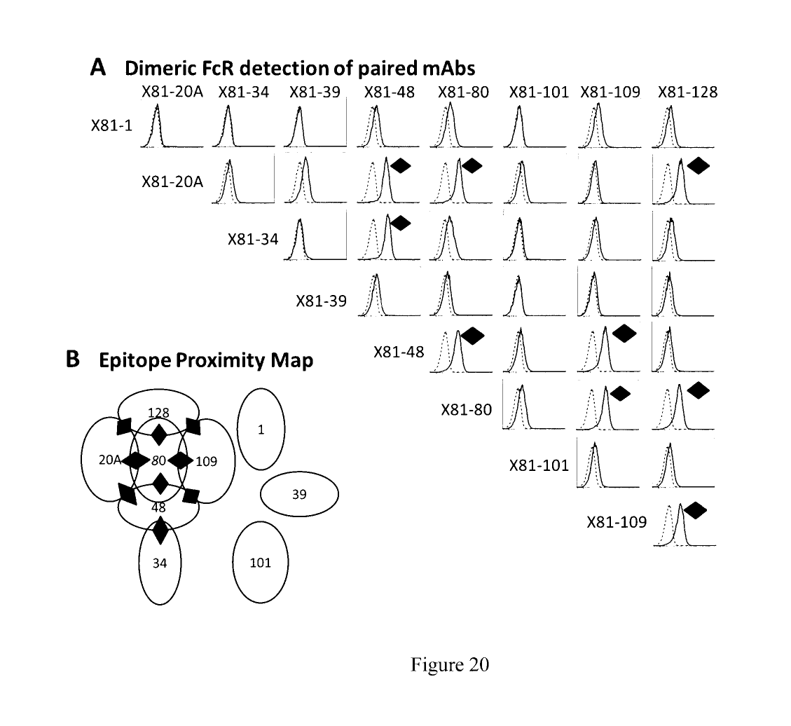

Pairwise combinations of the monoclonal antibodies were then investigated for their capacity to bind the dimeric FcγR and only certain pairs were able to clearly bind dimeric FcγR and are indicated by the solid diamonds in FIG. 20A. For example, the dimeric FcγR clearly bound to the paired combination of X81-128 with X81-20A but not the combination of X81-128 with X81-48. In contrast, dimeric FcγR bound to pairing of X81-48 with X81-34, demonstrating that only particular pairs of antibodies are sufficiently close to permit dimeric Fc receptor binding. This is also confirmed as the dimeric FcγR failed to bind to any IL-12 RB1 antibody paired with X81-1, X81-101 or X81-39. (FIG. 20A). Thus, on the basis of the evaluation of dimeric Fc receptor binding to combinations of antibodies, it is possible to build a map of the proximity or topology (FIG. 20B) that describes the relationships of the antibodies and their epitopes on a target entity. The epitope map reveals that X81-80 binds sufficiently close to X81-128, or the X81-20A, or X81-109, or X81-48, to allow dimeric Fc receptor binding. However, whilst X81-80 and X81-34 do not permit dimeric FcγRIIa binding, X81-34 with X81-48 does permit dimeric FcγRIIa binding. Clearly such an analysis provides the basis for defining combinations of antibodies that allow dimeric FcR binding and allows the further mapping of the spatial or topological relationships between distinct antibodies and their epitopes.

Accordingly, in this assay, despite extensive opsonisation of the target cells expressing IL-12RB1 (FIG. 19A), the dimeric FcR molecules do not bind to a significant level unless two or more antibodies are present against the target. The measurement of opsonisation alone, and more surprisingly, even with high levels of target opsonisation, is therefore not sufficient to predict an antibody or epitope topology/proximity that allows Fc receptor engagement. However, the use of recombinant soluble dimeric FcR in antibody mixtures, for example, pairs of antibodies, not only permits dimeric receptor binding but the pattern of binding provides information on the relationship or topology between the epitopes and the bound antibodies.

The data herein establishes that Fc receptor dimer binding correlates with Fc receptor dependent cell activation, as dimeric recombinant soluble FcγR binding to an opsonised target correlates with NK cell activation caused by the same immune complex. Further, the dimeric FcR binding to only selected combinations of antibodies indicates that this combination of antibodies are likely to be more effective in the induction of effector function than either antibody alone. Accordingly, the effectiveness of therapeutic antibodies may depend on Fc engagement of Fc receptors of effector cells such as NK cells. The dimeric FcR provide a method to identify antibodies or combinations of antibodies that have an appropriate Fc topology to mediate FcγR binding and effector cell activation. Furthermore, this may particularly be useful to identify where a target molecule is of low abundance on the surface of a target cell and where a single monoclonal antibody may be insufficient to induce Fc receptor dependent cell activation. In this situation, combinations of antibodies recognising different epitopes in close proximity may allow dimeric receptor binding. Accordingly, the Fc binding multimers may provide a means to select combinations of antibodies that target a particular antigen for further industrial development or for patient therapy.

The data herein also establishes that Fc binding multimers may be used to build of an epitope or topology map, which provides a guide to the relationship of the epitopes on an antigenic target. This in turn can provide a means for distinguishing antibodies of different epitope specificity in close proximity.

Further, this method could be adapted build a proximity map using antibodies against entirely different molecules (for example IL-12 RB1 and another surface molecule for example IL-23R specific chain or CD3 of the T cell antigen receptor), and such maps could provide information about the relationship of these molecules on the cell surface.

### Example 5 Dimeric rsFcγRIIIa Binding to ICs of IvIg Opsonized HA of A(H1N1)pdm09 Virus Correlates with Hemagglutinin Inhibition Titer

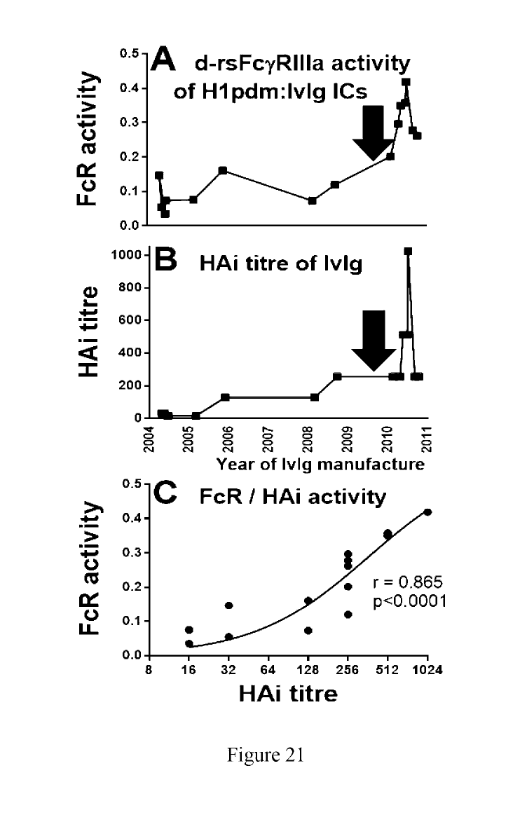

Fifteen commercial Mg preparations (prepared from 2004 to 2010; IvIg:Sandoglobulin were from bioCSL, Australia) were diluted to 500, 250, and 125 μg/ml (n=4, 5, 4 experiments respectively) and used to opsonize influenza HA from A(H1N1) pdm09 virus (Sinobiological, Shanghai, China). The dimeric rsFcγRIIIa Val158 binding activity of these ICs was determined as described above and analysed across experiments by normalising A450 nm values to that for Mg 1848 at 250 μg/ml as shown in FIG. 21(A). The arrow in FIG. 21A and FIG. 21B shows the approximate time of emergence of the A(H1N1)pdm09 influenza virus in the population. The HA inhibition titre (HAi) for these Mg preparations is shown in FIG. 21B, with the data obtained from Table 1 of Jegaskanda, et al. (2014). FIG. 21 C shows the correlation between FcR activity and HA inhibition titre, the values were correlated using non-parametric spearman analysis.

These results show that higher dimeric rsFcγRIIIa binding activity was found for Mg manufactured in 2010, indicating an increased functional capacity of antibodies specific for the HA of influenza virus A(H1N1)pdm09 was detected after the emergence of the influenza pandemic in 2009. It is noteworthy that the elevated dimeric rsFcγRIIIa activity was transient, peaking and declining in 2010.

### Example 6 Selective Binding of Dimeric but not Monomeric Recombinant Soluble FcγR to IgG Immune Complexes

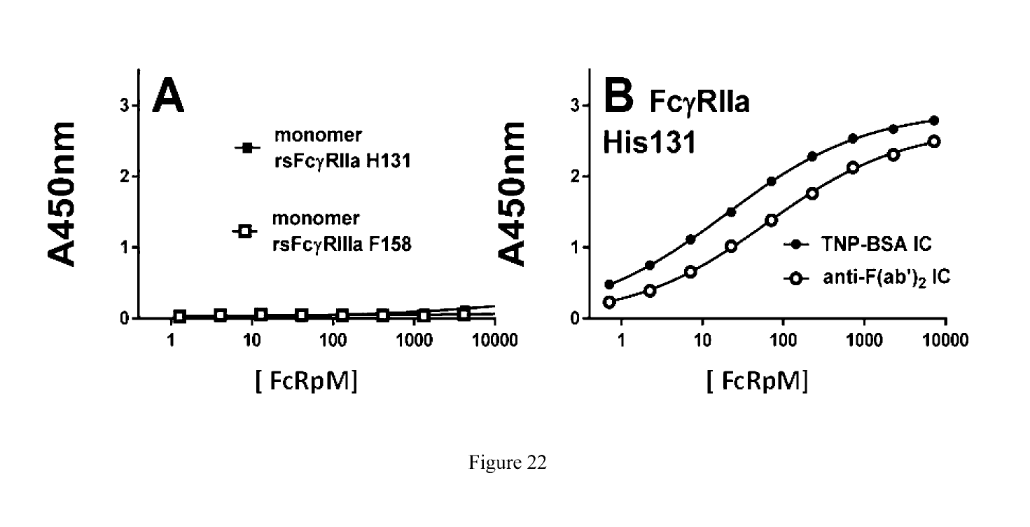

Binding assays were conducted as described in Example 1, with human IgG1 (1 ng per ml) formed into immune complexes by binding to immobilised TNP BSA. Neither recombinant soluble monomeric FcγRIIa H131 nor FcγRIIIa F158 bound to the immune complexes as shown in FIG. 22A. However, dimeric recombinant soluble FcγRIIa bound to the same IgG1 TNP BSA complexes (closed symbols) and also to human IgG1 immune complexes formed by capture with plate bound Fab′ 2 anti-human Fab′ 2 (open symbols) as shown in FIG. 22 B. Thus, the binding to immune complexes of dimeric or oligomeric FcγR but not monomeric FcγR indicates that binding is dependent on avidity. Of note, oligomerisation of monomeric recombinant soluble receptors FcγR with streptavidin also resulted in binding to immune complexes (as shown in FIGS. 4, 5, 6).

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

Acharya, P et al., J Virol 88:12895-12906 (2014).
An, Z et al., mAbs 1:572-579 (2009).
Baruah, K et al., J Mol Biol 420:1-7 (2012).
Bruhns P et al., Blood 113(16):3716-3726 (2009).
Cameron, F and P L McCormack, Drugs 74:147-154 (2014).
Center, R J et al., Vaccine 27:6605-6612 (2009).
Derer, S et al., mAbs 6:409-421 (2014).
Dilillo Di and J V Ravetch, Cancer Immuno Res 3(7):704-713 (2015).
Evans M J, et al. J Immunol Methods; 184(1): 123-38 (1995).
Ferrara, C et al., Biotechnol Bioeng 93: 851-861 (2006).
Ferrara, C et al., Proc Nat Acad Sci USA 108: 12669-12674 (2011).
Glanville, J et al., Proc Nat Acad Sci USA 106:20216-20221 (2009).
Herter, S et al., Molec Cancer Therap 12:2031-2042 (2013).
Hibbs M L et al., Proc Natl Acad Sci USA 85(7):2240-2244 (1988).
Ierino, F et al, J Immunology 150:1794-1803 (1993).
Hogarth, P M and G A Pietersz, Nature Reviews. Drug Discovery 11: 311-331(2012).
Howarth, M J et al., Proc Nat Acad Sci USA 102:7583-7588 (2005).
Huber et al., J Virol 84:10700-10707 (2010).
Hulett, M D et al. J Biol Chem. 269(21):15287 (1994).
Hulett, M D et al. J Biol Chem. 270(36):21188 (1995).
Hulett, M D. Mol Immunol. 35(14-15):989 (1998).
Iida, S et al., Clin Cancer Res 12:2879-2887 (2006).
Midge, Tel al., Expert Opin on Biol Ther 14:1507-1517 (2014).
Jegaskanda et al. J Infect Dis 210: 1811-1822 (2014).
Klein, C et al., mAbs 5:22-33 (2013).
Lux, A et al., J Immunol 190:4315-4323 (2013).
Maxwell et al., Nat Struct Riot 6:437-442 (1999).
Moldt, B et al., J Virol 86:6189-6196 (2012).
Musolino, A et al., J Clin Oncol 26:1789-1796 (2008).
Patel, D et al., J Immunol 184:6283-6292 (2010).
Ramsland P A et al., J Comput Aided Mol Des 11(5):453-461 (1997).
Rogers, K A and J A Jones, Drugs of Today (Barcelona, Spain: 1998) 50:407-419 (2014).
Shinkawa, T et al., J Biol Chem 278:3466-3473 (2003).
Smith, T F and M S Waterman, Adv Appl Math 2:482-489 (1981).
Trist et al. J Immunol 192(2):792-803 (2014).
Tamm, A et al. J Biol Chem. 271(7):3659 (1996).
Petersen T N et al., Nat Methods 8:785-786 (2011).
Plosker, G L and D P Figgitt. Drugs 63:803-843 (2003).
Weng, W K and R Levy. J Clin Oncol 21:3940-3947 (2003).
Wines et al. J Biol Chem; 279(25): 26339-45 (2004)
Zalevsky, J et al., Blood 113:3735-3743 (2009).

What is claimed is:

1.-7. (canceled)

8. A method of assessing in vitro the FcR binding activity of an antibody comprising an Fc region or fragment thereof, the method comprising the following steps:
a. providing an at least one Fc binding multimer comprising at least two Fc binding regions;
b. incubating the antibody with the at least one Fc binding multimer under conditions suitable to permit specific binding of the Fc binding regions to a Fc region or fragment thereof; and
c. identifying the magnitude of FcR binding activity of the antibody with the Fc binding multimer using a label agent;
wherein the magnitude of FcR binding activity identified in step (c) provides an assessment of the FcR binding activity of the antibody.

9. The method of claim 8, wherein the Fc binding regions are derived from a Fcγ receptor (FcγR) selected from the group consisting of FcγRIIa-His131, FcγRIIa-Arg131, FcγRIIa-Pro131, FcγRIIb, FcγRIIIa-Val158, and FcγRIIIa-Phe158.

10. The method of claim 8, wherein the Fc binding multimer consists of a Fc binding peptide comprising at least two Fc binding regions joined by a peptide linker domain.

11. The method of claim 8, wherein the Fc binding multimer consists of at least two Fc binding peptides each comprising an at least one Fc binding region, wherein each Fc binding peptide is bound to a first partner of a specific binding pair such that when a second partner of the specific binding pair is present, at least two of the first partners each bind with the second partner of the specific binding pair to form the Fc binding multimer.

12. The method of claim 8, wherein step (b) further comprises immobilising the antibody by binding with an immobilised specific antigen or an immobilised immunoglobulin or fragment thereof which specifically binds with the antibody, prior to incubating the antibody with the at least one Fc binding multimer.

13. The method of claim 8, wherein step (b) further comprises incubating the antibody with a cell bound specific antigen, or a cell-fragment-bound specific antigen to bind the antibody to the cell or cell fragment, prior to incubating the antibody with the at least one Fc binding multimer.

14. The method of claim 8, further comprising comparing the magnitude of FcR binding activity between the Fc binding multimer and the antibody with the magnitude of binding between the Fc binding multimer and one or more control antibodies.

15. The method of claim 14, wherein an increased magnitude of FcR binding activity of the antibody compared to a control antibody demonstrates that the Fc region of the antibody has stronger binding activity for the Fc binding region than the control antibody, a decreased magnitude of binding of the antibody compared to a control antibody demonstrates that the antibody has weaker binding activity for the Fc binding region than the control antibody, or unchanged or similar magnitude of binding of the Fc region of an antibody compared to a control antibody demonstrates that the antibody has the same or similar binding activity for the Fc binding region as the control antibody.

16. The method of claim 8, further comprising comparing the magnitude of FcR binding activity between the antibody and a plurality of different Fc binding multimers to identify which Fc binding regions the antibody preferentially binds with, and wherein the identifying of preferential binding to a Fc binding region indicates whether the antibody will in use induce a desired immune response outcome.

17. The method of claim 8, further comprising comparing the magnitude of FcR binding activity between the Fc binding multimer and a panel of different antibodies each specific for a same specific antigen to identify an antibody that preferentially binds to a Fc binding multimer comprising at least two Fc binding regions derived from a selected FcR.

18. The method of claim 8, wherein the FcR binding activity of at least two target antibodies are simultaneously assessed to map the proximity of the at least two antibodies when bound to a target antigen.

19. The method of claim 8, wherein step (b) further comprises incubating the antibody with an antigenic target or target cell under conditions suitable to permit opsonisation of the antigenic target or target cell with the antibody, and then incubating the antibody with the at least one Fc binding multimer, wherein the FcR binding activity of the antibody indicates whether opsonisation of the antigenic target or target cell with the antibody is likely to induce a cellular induced response including antibody dependent cellular cytoxicity (ADCC), antibody dependent cellular phagocytosis degranulation, mediator release, cytokine production and antigen presentation, or lack thereof.

20. A method of selecting an at least one antibody or fragment thereof having an Fc region with a desired FcR binding activity from a panel of antibodies or fragments thereof, the method comprising
a. providing an Fc binding multimer comprising at least two Fc binding regions, wherein the Fc binding regions are derived from a pre-selected Fc receptor (FcR);
b. separately incubating each of the panel of antibodies or fragments thereof with the Fc binding multimer under conditions suitable to permit specific binding of an antibody with a Fc region having the preferred FcR binding activity with the Fc binding multimer;
c. detecting magnitude of binding of each of the panel of antibodies to the Fc binding multimer; and
d. selecting from the panel of antibodies an antibody having a high magnitude of binding with the Fc multimer;
wherein the selected antibody has the desired FcR binding activity.


SEQUENCE LISTING (23 sequences)

SEQ ID NO: 1 - protein, 317 aa, homo sapiens
  signal sequence (1)..(35)
  N terminus of mature peptide (36)..(36)
  His 131 (167)..(167)
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 2 - protein, 317 aa, homo sapiens [sequence omitted]

SEQ ID NO: 3 - protein, 307 aa, Macaca nemestrina
METQMSQNVCPGNLWLLQPLTVLLLLASADSQTAPPKAVLKLEPPWINVLREDSVTLTCGGAHSPDSDSTQWFHNGNLIPTHTQPSYRFKANNNDSGEYRCQTGRTSLSDPIHLTVLSEWLALQTPHLEFREGETIMLRCHSWKDKPLIKVTFFQNGISKKFSHMDPNFSIPQANHSHSGDYHCTGNIGYTPYSSKPVTITVQVPSVGSSSPMGIIVAVVTGIAVAAVVAAVVALIYCRKKRISANSTDPVKAARFEPLGHQTIALRKRQLEETNNDYETVDGGYMTLNPRAPTDDDRNIYLALSPN

SEQ ID NO: 4 - protein, 307 aa, Macaca nemestrina
METQMSQNVCPGNLWLLQPLTVLLLLASADSQTAPPKAVLKLEPPWINVLREDSVTLTCGGAHSPDSDSTQWFHNGNLIPTHTQPSYRFKANNNDSGEYRCQTGRTSLSDPIHLTVLSEWLALQTPHLEFREGETIMLRCHSWKDKPLIKVTFFQNGISKKFSPMDPNFSIPQANHSHSGDYHCTGNIGYTPYSSKPVTITVQVPSVGSSSPMGIIVAVVTGIAVAAVVAAVVALIYCRKKRISANSTDPVKAARFEPLGHQTIALRKRQLEETNNDYETVDGGYMTLNPRAPTDDDRNIYLALSPN

SEQ ID NO: 5 - protein, 309 aa, Homo sapiens
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 6 - protein, 312 aa, Macaca nemestrina
MGILSFLPVLATESDWADCKSPQPWGHMLLWTAVLFLAPVAGTPAPPKAVLKLEPPWINVLREDSVTLTCGGAHSPDSDSTQWFHNGNLIPTHTQPSYRFKANNNDSGEYRCQTGRTSLSDPVHLTVLSEWLALQTPHLEFREGETIMLRCHSWKDKPLIKVTFFQNGISKKFSHMDPNFSIPQANHSHSGDYHCTGNIGYTPYSSKPVTITVQVPSMGSSSPIGIIVAVVTGIAVAAIVAAVVALIYCRKKRISALPGNPECREMGETLPEKPANPTNPDEADKVGAENTITYSLLMHPDALEEPDDQNRV

SEQ ID NO: 7 - protein, 254 aa, Homo sapiens [sequence omitted]

SEQ ID NO: 8 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 9 - protein, 15 aa, Artificial Sequence
  AviTag biotinylation sequence
GLNDIFEAQKIEWHE

SEQ ID NO: 10 - protein, 238 aa, Artificial Sequence
  Human rsFcgammaRIIa H131 monomer fusion peptide
METQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGPGSSSHHHHHHPGGGLNDIFEAQKIEWHE

SEQ ID NO: 11 - protein, 418 aa, Artificial
  Human rsFcgammaRIIa H131 dimer fusion peptide
METQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPVAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGPGSSSHHHHHHPGGGLNDIFEAQKIEWHE

SEQ ID NO: 12 - protein, 225 aa, Artificial
  Human rsFcgammaRIIa R131 monomer fusion peptide
MVLSLLYLLTALPGILSAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPGSSSHHHHHHPGGGLNDIFEAQKIEWHE

SEQ ID NO: 13 - protein, 405 aa, Artificial
  Human rsFcgammaRIIa R131 dimer fusion peptide
MVLSLLYLLTALPGILSAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPGSSSHHHHHHPGGGLNDIFEAQKIEWHE

SEQ ID NO: 14 - protein, 225 aa, Artificial
  Human rsFcgammaRIIb monomer fusion peptide
MVLSLLYLLTALPGILSAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPGSSSHHHHHHPGGGLNDIFEAQKIEWHE

SEQ ID NO: 15 - protein, 405 aa, Artificial
  Human rsFcgammaRIIb dimer fusion peptide
MVLSLLYLLTALPGILSAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGPGSSSHHHHHHPGGGLNDIFEAQKIEWHE

SEQ ID NO: 16 - protein, 228 aa, artificial
  Human rsFcgammaRIIa V158 monomer fusion peptide
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPGPGSSSHHHHHHPGGGLNDIFEAQKIEWHE

SEQ ID NO: 17 - protein, 410 aa, Artificial
  Human rsFcgammaRIIIa V158 dimer fusion peptide
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGPSMGSSSPGPGSSSHHHHHHPGGGLNDIFEAQKIEWHE

SEQ ID NO: 18 - protein, 228 aa, Artificial
  Human rsFcgammaRIIIa F158 monomer fusion peptide
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPGPGSSSHHHHHHPGGGLNDIFEAQKIEWHE

SEQ ID NO: 19 - protein, 410 aa, Artificial
  Human rsFcgammaRIIIa F158 dimer fusion peptide
MVLSLLYLLTALPGISTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPSEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGPSMGSSSPGPGSSSHHHHHHPGGGLNDIFEAQKIEWHE

SEQ ID NO: 20 - protein, 242 aa, Artificial
  Macaque rsFcgammaRIIa H131 monomer fusion peptide
METQMSQNVCPGNLWLLQPLTVLLLLASADSQTAPPKAVLKLEPPWINVLREDSVTLTCGGAHSPDSDSTQWFHNGNLIPTHTQPSYRFKANNNDSGEYRCQTGRTSLSDPIHLTVLSEWLALQTPHLEFREGETIMLRCHSWKDKPLIKVTFFQNGISKKFSHMDPNFSIPQANHSHSGDYHCTGNIGYTPYSSKPVTITVQVPSVGSSSPGPGSSSHHHHHHPGGGLNDIFEAQKIEWHE

SEQ ID NO: 21 - protein, 242 aa, Artificial
  Macaque rsFcgammaRIIa P131monomer fusion peptide
METQMSQNVCPGNLWLLQPLTVLLLLASADSQTAPPKAVLKLEPPWINVLREDSVTLTCGGAHSPDSDSTQWFHNGNLIPTHTQPSYRFKANNNDSGEYRCQTGRTSLSDPIHLTVLSEWLALQTPHLEFREGETIMLRCHSWKDKPLIKVTFFQNGISKKFSPMDPNFSIPQANHSHSGDYHCTGNIGYTPYSSKPVTITVQVPSVGSSSPGPGSSSHHHHHHPGGGLNDIFEAQKIEWHE

SEQ ID NO: 22 - protein, 253 aa, Artificial
  Human rsFcgammaRIIb monomer fusion peptide
MGILSFLPVLATESDWADCKSPQPWGHMLLWTAVLFLAPVAGTPAPPKAVLKLEPPWINVLREDSVTLTCGGAHSPDSDSTQWFHNGNLIPTHTQPSYRFKANNNDSGEYRCQTGRTSLSDPVHLTVLSEWLALQTPHLEFREGETIMLRCHSWKDKPLIKVTFFQNGISKKFSHMDPNFSIPQANHSHSGDYHCTGNIGYTPYSSKPVTITVQVPSMGSSSPGPGSSSHHHHHHPGGGLNDIFEAQKIEWHE

SEQ ID NO: 23 - protein, 11 aa, artificial
  Linker peptide
VPSMGSSSPVA